SEQUENCE LISTING (28 sequences)

SEQ ID NO: 1 - DNA, 687 bp, Mus musculus
  CDS (1)..(687)
  misc_feature (157)..(222): Encodes TM domain
  misc_feature (223)..(687): Encodes IC domain
  misc_feature (223)..(303): Encodes Chopper domain
  misc_feature (454)..(663): Encodes death domain
  misc_feature (481)..(534): Encodes p75 binding domain
  misc_feature (673)..(683): Encodes PDZ domain binding site
atgctttataacgtcagcaaaggtgtggtctattcagatacagccctgcaggggcaggacggggacagggaaggaatgtgggtaggagctgggggagccctagcccccaatacctcctccctatttccccctgagcctccaggggcctcgagcaacatcattcctgtctactgtgctctcctagctacagtgatccttggtctgctggcctatgtggccttcaaatgctggcgctcacataagcaaaggcaacagttggctaaagctcggactgtagagctaggggaccctgacagggaccagaggcgtggtgacagcaacgtcttcgtggactctcctcctagtctggagccctgtattcccagccagggaccacacccggaccttggctgccagctttacctgcatattccacagcagcagcaggaggaagtccagcggctcctgatgatgggtgagccagccaagggctggcaggagctggcaggccacctcggctaccaagctgaggctgtggaaaccatggcctgtgaccaaatgccagcctataccctgctaaggaactgggctgcccaagaaggcaatagagctaccctcagagtgctggaggatgctctggctgccataggccgagaagatgtggtccaggttttgagctcgccagctgagagctcctcggtggtgtga

SEQ ID NO: 2 - protein, 228 aa, Mus musculus
MLYNVSKGVVYSDTALQGQDGDREGMWVGAGGALAPNTSSLFPPEPPGASSNIIPVYCALLATVILGLLAYVAFKCWRSHKQRQQLAKARTVELGDPDRDQRRGDSNVFVDSPPSLEPCIPSQGPHPDLGCQLYLHIPQQQQEEVQRLLMMGEPAKGWQELAGHLGYQAEAVETMACDQMPAYTLLRNWAAQEGNRATLRVLEDALAAIGREDVVQVLSSPAESSSVV

SEQ ID NO: 3 - DNA, 687 bp, Rattus norvegicus
  CDS (1)..(687)
  misc_feature (157)..(222): Encodes TM domain
  misc_feature (223)..(687): Encodes IC domain
  misc_feature (223)..(303): Encodes Chopper domain
  misc_feature (451)..(534): Encodes p75 binding domain
  misc_feature (454)..(663): Encodes death domain domain
  misc_feature (673)..(684): Encodes PDZ domain binding site
atgcttcataacgtcagcaaaggtgtggtgtattcagatacagccctgaaggggcaggatggggacagggaaggaatgtgggtaggagctgggggagccctagcccccaatacctcctccctatttccccctgagcctccaggggcctcaagcaacatcattcctgtctactgtgctctcctagctactgaagtccttggtctgcttgcctatgtggccttcaaatgctggcgctcacgtaagcaaagacaacagttggctaaagctcggactgtagagctgggggaccctgaccgggaccaaaggcatggtgacagcagtgtcttcgtggattctcctcatggtctggagccctgtattcccagccagggaccacatgcagaccttggctgccgactttacctgcatattccacagcagcagcaggaggaagtccagcggctcttgatattgggggaacctgccaagggctggcaggggctggcaggccaactgggctaccaagctgaagctgtggaaaccatggcctgtgaccaggacccagcctatgccctgctaagggactgggctgctcaagaaggcagtggagcaaccctcagagtgctagaggatgctctgactgccataggccgagaagatgtggtccaggttttgagctcaccagctgagggctgctctgtggtgtga

SEQ ID NO: 4 - protein, 228 aa, Rattus norvegicus
MLHNVSKGVVYSDTALKGQDGDREGMWVGAGGALAPNTSSLFPPEPPGASSNIIPVYCALLATEVLGLLAYVAFKCWRSRKQRQQLAKARTVELGDPDRDQRHGDSSVFVDSPHGLEPCIPSQGPHADLGCRLYLHIPQQQQEEVQRLLILGEPAKGWQGLAGQLGYQAEAVETMACDQDPAYALLRDWAAQEGSGATLRVLEDALTAIGREDVVQVLSSPAEGCSVV

SEQ ID NO: 5 - DNA, 690 bp, Bos taurus
  CDS (1)..(690)
  misc_feature (484)..(537): Encodes p75 binding domain
atgctgcagaactccagccacagggaagccatggtccatgtggataagacccggagggagcaggacagggaaggggtgtgggcagcggcagggggagccctggcccccagcacctcctccccattctcccctgagcccccaggggcctcaggcagcatcatccctgtctactgtgccctcctggccaccgtggtccttggtctgcttgcctacgtggtcttcaagtgctggcgctcacgcaaacacaggcagcagctggccaaagctcggaccgcagagctgggggccctcagcagggaccagctccatggggacagcagtgtctttcgggactctccagctggcctggagccctgtgcccccagccaagggccgccgcctgaacttggctgccggctctacctccaccttcctcggcagcagcaggaggaagtggaacggctcctggaggtgtcgggtgaaccggccaacggctggcggggcctggcgggccgcctgggctaccaggctgaggcagtggagaccatggcccggagcccagggcccgcctctgccctgctgagggactgggctgtccaggaaggcagtggcgccaccctcagagcgctagcagacgcccttgctgccatgggccgtgaagacgtgattcgggctctgagctccccggctgagggttgctctgtggtgtga

SEQ ID NO: 6 - protein, 229 aa, Bos taurus
MLQNSSHREAMVHVDKTRREQDREGVWAAAGGALAPSTSSPFSPEPPGASGSIIPVYCALLATVVLGLLAYVVFKCWRSRKHRQQLAKARTAELGALSRDQLHGDSSVFRDSPAGLEPCAPSQGPPPELGCRLYLHLPRQQQEEVERLLEVSGEPANGWRGLAGRLGYQAEAVETMARSPGPASALLRDWAVQEGSGATLRALADALAAMGREDVIRALSSPAEGCSVV

SEQ ID NO: 7 - DNA, 732 bp, Sus scrofa
  CDS (1)..(732)
  misc_feature (526)..(579): Encodes p75 binding domain
atgcagcccctcggagcactgctggctggtagaaccatgcctcacaactccagccacagggaagacatgggccatgtggataagccccagaaggagcaggatgggaagaggggaggggcatggttaggggcagggggagccctggcccccagtacctcctccccattcccccctgagcctccaggggcttcaggcagcatcatccctgtctactgtgccctcctagccactgtggtcctcggtctgcttgcttatgtggccttcaagtgctggcgctcacataaacaaaggcagcaactggccaaggctcgaactgcggagctggggggcctcaaaagggaccatatgcctggggatagcagtgtcttccaggactctcctagcggcctggagccctgtgtccccagccaggggccgcatcctgaacttggctgccggctctacctccatatccctccgcagcagcgacaggaagtggagcagctcttggaggcctcaggtgaacctgacaaaggctggcagggcctggcaggctacctgggctaccaagctgaggctgtggagaccatggcccggagccaggtgccagcctacactctgctgagggactgggccatccgggaaggcagcggtgccaccctcagggtgctagcagatgccctggctgccatgggccgtgaagatgtggtccaggtcctggatcccccagctgagggctgctctgtggtgtga

SEQ ID NO: 8 - protein, 243 aa, Sus scrofa
MQPLGALLAGRTMPHNSSHREDMGHVDKPQKEQDGKRGGAWLGAGGALAPSTSSPFPPEPPGASGSIIPVYCALLATVVLGLLAYVAFKCWRSHKQRQQLAKARTAELGGLKRDHMPGDSSVFQDSPSGLEPCVPSQGPHPELGCRLYLHIPPQQRQEVEQLLEASGEPDKGWQGLAGYLGYQAEAVETMARSQVPAYTLLRDWAIREGSGATLRVLADALAAMGREDVVQVLDPPAEGCSVV

SEQ ID NO: 9 - DNA, 1164 bp, Xenopus laevis
  CDS (1)..(1164)
atggataagaggggccccatcgtaaccctttgcctgctgctgctgatctccaagatatcggcagaagacgtttgcgagagtggcctctacacaaacagcggcaaatgctgttccttgtgcccagcgggattcggggtggtggttccctgcggagattcagatactaagtgtgaaccctgcatagagaactctactttctctgatgtcagaagcgccaaggcaaagtgccagccatgtttcacctgccaaagtccgtctctgacgctagaatccaactgtactcgcgagcaggataccgtatgtcgttgcccagagaggcagtatctggacagcaatggcatttgtctcccatgccagctatgctctaaggggcatggagttgtctctcaatgtactcacaataaaaacactgtgtgccaattgtgctcaagcggcttctactcagaggtgaaaagtagtgaatccccttgcctgccctgccggactgaatgcaaagaaaccgaggtgcagataggagattgtgtgccgcaacatgacattctgtgcatggataaagacgttcccatcctcaaacgcactgaaggtggtgagaacggcacttctgcggggtccccccacttcatcccacaggacaacagcaagaacatcattcccgtgtactgctccatcttagcagctgtggtggttggacttattgcttacgtggcgtttaagtgctatactacctgcaaacagaagaagcagttggccaaagccagagctggagaattggctacatccaccgagggcgagaagttgcacaatgatagcggcgtcttccttgatacacacagcctccaggaacccaatcatctgagcaaagccaagattgaaccaaagctctacattaacttgccacctcacaagcaaagtgaagtggagcgcctgttggcagacaccagccttgggaaagattggcagcgcctcgccagcctcttgggatacgaggaggaaaccattgacacattcgggcgaggagaggatccagtacacactcttctcactgactggtcttccaaagagagctcaaccctggaggtcctatgtgctgctttggtcaacatggaacgggcagacgtggtggaaaatctgaacagcacaaacgatgccagctccgtggtgtaa

SEQ ID NO: 10 - protein, 387 aa, Xenopus laevis
MDKRGPIVTLCLLLLISKISAEDVCESGLYTNSGKCCSLCPAGFGVVVPCGDSDTKCEPCIENSTFSDVRSAKAKCQPCFTCQSPSLTLESNCTREQDTVCRCPERQYLDSNGICLPCQLCSKGHGVVSQCTHNKNTVCQLCSSGFYSEVKSSESPCLPCRTECKETEVQIGDCVPQHDILCMDKDVPILKRTEGGENGTSAGSPHFIPQDNSKNIIPVYCSILAAVVVGLIAYVAFKCYTTCKQKKQLAKARAGELATSTEGEKLHNDSGVFLDTHSLQEPNHLSKAKIEPKLYINLPPHKQSEVERLLADTSLGKDWQRLASLLGYEEETIDTFGRGEDPVHTLLTDWSSKESSTLEVLCAALVNMERADVVENLNSTNDASSVV

SEQ ID NO: 11 - DNA, 697 bp, Homo sapiens
  CDS (1)..(246)
atgattcacaactctagccacagcaaagtcctggcctatgtgggtaagatcctgaggaggcaggagagggacagggaaggagtatgggtaggggcaggaggagccctggcccccaatacctcctcccctttcctcctggagtctccaagaggtctcaggcagcatcatcccagtctactgtacccttctggccactgtggtcctcggtctgcttgcctacatggccttcaagtgctggcgctcacataaacaaagacagcagctggccaaagctcgaactgcagagctgggaggtctcaacaaggaccagatgcatggggatagcagtgtcttcctggactctcctagcagtctggagccctgtgcccccagccagggaccacatcctgagcttgggtgccgacttcaccttgatctccctcggcagcaccaggagaaagtggagcggctgttagaggtgtcacgtgaacctgacaagggctggcggggactggcagaccatctgggctaccaagctgaggctgtagaaatcatggcccaaggccaggtgtgagcctacacactgctgagggactgggctatccaagaagacagtggtgccacccacaaggtgctagagaatgccctggttgccatgggccaggaagatgtggtccaggtcctgggcccccaagctgagggctgttctgtggtgtga

SEQ ID NO: 12 - protein, 82 aa, Homo sapiens
MIHNSSHSKVLAYVGKILRRQERDREGVWVGAGGALAPNTSSPFLLESPRGLRQHHPSLLYPSGHCGPRSACLHGLQVLALT

SEQ ID NO: 13 - DNA, 697 bp, Pan troglodytes
  CDS (1)..(246)
atgattcacaactctagccacagcaaagtcctggcctatgtgggtaagatcctgaggaggcaggagagggacagggaaggagtatgggtaggggcagggggagccctggcccccaatacctcctcccctttactcctggagtctccaaggggtctcaggcagcatcatcccagtctactgtaccctcctggccactgtggtcctcggtctgcttgcctacatggccttcaagtgctggcgctcacataaacaaagacagcagctggccaaagctcgaactgcagagctgggaggtctcaacaaggaccagatgcatggggatagcagtgtcttcctggactctcctagcagtctggagccctgtgcccccagccagggaccacatcctgagattgggtgccgactttaccttgatctccctcggcagcaccaggagaaagtggagcggctgttagaggtgtcacgtgaacctgacaagggctggcggggactggcagaccatctgggctaccaagctgaggctgtagaaatcatggcccaaggccaggtgtgagcctacacactgctgagggactgggctatccaagaagacagtgttgccacccacaaggtgctagagaatgccctggttgccatgggccaggaagatgtggtccaggtcctgggcccccaagctgaggactgctctgtggtgtga

SEQ ID NO: 14 - protein, 82 aa, Pan troglodytes
MIHNSSHSKVLAYVGKILRRQERDREGVWVGAGGALAPNTSSPLLLESPRGLRQHHPSLLYPPGHCGPRSACLHGLQVLALT

SEQ ID NO: 15 - DNA, 691 bp, Macaca mulatta
  CDS (1)..(240)
atgcttcacaactctagccacagcaaaggcctggcctatgtgggtaagatcctgaggaggcaggagagggacagggaaggagtatgggtaggggcagggggagccctgtaccccaatacttcccctttcctcctggagcctccagtgtctcaggcgcatcatcccagtctactgtaccctcctggccactgtggtcctcggtctgcttgcctatgtggccttcaagtgctggcgctcacatagacaaagacagcagctggccaaagctcgaactgcagagctgggaggtctcaacaaggaccagatgcatggggatagcagtgtcttcctggactctcctagcagtctggaaccctgtgcctccaaccagggaccacatcctgagtttgggtgccaactttaccttgatctccctcggcagcaccaggagaaagtggagcggctgctagaggtgtcagttgaaactgacaagggctggcggggactggcagaccatctgggctaccaggctgaggctgtagaaatcatggcccaaggccaggtgcgagcctacacactgctgagggactgggctatccaagaaggcagtggcgccacccacaaggtgctagagaatgccctggttgccatgggccaggaagatgtggtccaggtcctgggcccccaagctgagggctgctctgtggcatta

SEQ ID NO: 16 - protein, 80 aa, Macaca mulatta
MLHNSSHSKGLAYVGKILRRQERDREGVWVGAGGALYPNTSPFLLEPPVSQAHHPSLLYPPGHCGPRSACLCGLQVLALT

SEQ ID NO: 17 - DNA, 1278 bp, Rattus norvegicus
  CDS (1)..(1278)
  misc_feature (1078)..(1131): Encodes p45 binding domain
atgaggagggcaggtgctgcctgcagcgccatggaccggctgcgcctgctgctgctgctgattctaggggtgtcctctggaggtgccaaggagacatgttccacaggcctgtacacccacagcggagagtgctgcaaagcctgcaacttgggcgaaggcgtggcccagccctgcggagccaaccagaccgtgtgtgaaccctgcctggacagtgttacattctccgatgtggtgagcgccactgagccgtgcaagccgtgcaccgagtgcctgggcctgcagagcatgtccgctccctgtgtggaggcagacgatgcagtgtgcagatgtgcctatggctactaccaggacgaggagactggccactgtgaggcttgcagcgtgtgcgaggtgggctcgggactcgtgttctcctgccaggacaaacagaacacagtgtgtgaagagtgcccagagggcacatactcagacgaagccaaccacgtggacccgtgcctaccctgcacggtgtgcgaggacactgagcgccagttacgcgagtgcacgccctgggctgatgctgaatgcgaagagatccctggtcgatggatcccaaggtctacgcccccggagggctccgacagcacagcgcccagcacccaggagcctgaggttcctccagagcaagaccttgtacccagtacagtggcggatatggtgaccactgtgatgggcagctcccagcctgtagtgacccgcggcaccaccgacaacctcattcctgtctattgctccatcttggctgctgtggtcgtgggccttgtggcctatattgctttcaagaggtggaacagctgcaaacaaaataaacaaggcgccaacagccgccccgtgaaccagacgcccccaccggagggagagaaactgcacagcgacagtggcatctctgtggacagccagagcctgcacgaccagcagacccatacgcagactgcctcaggccaggccctcaagggtgatggcaacctctacagtagcctgcccctgaccaagcgtgaggaggtagagaaactgctcaacggggatacctggcgacatctggcaggcgagctgggttaccagcctgaacatatagactcctttacccacgaggcctgcccagtgcgagccctgctggccagctggggtgcccaggacagtgcaacgcttgatgcccttttagccgccctgcgacgcatccagagagctgacattgtggagagtctatgcagcgagtccactgccacatccccagtgtga

SEQ ID NO: 18 - protein, 425 aa, Rattus norvegicus
MRRAGAACSAMDRLRLLLLLILGVSSGGAKETCSTGLYTHSGECCKACNLGEGVAQPCGANQTVCEPCLDSVTFSDVVSATEPCKPCTECLGLQSMSAPCVEADDAVCRCAYGYYQDEETGHCEACSVCEVGSGLVFSCQDKQNTVCEECPEGTYSDEANHVDPCLPCTVCEDTERQLRECTPWADAECEEIPGRWIPRSTPPEGSDSTAPSTQEPEVPPEQDLVPSTVADMVTTVMGSSQPVVTRGTTDNLIPVYCSILAAVVVGLVAYIAFKRWNSCKQNKQGANSRPVNQTPPPEGEKLHSDSGISVDSQSLHDQQTHTQTASGQALKGDGNLYSSLPLTKREEVEKLLNGDTWRHLAGELGYQPEHIDSFTHEACPVRALLASWGAQDSATLDALLAALRRIQRADIVESLCSESTATSPV

SEQ ID NO: 19 - protein, 22 aa, Artificial Sequence
  Consensus sequence for non-primate, mammalian p45 TM domain
  MISC_FEATURE (12)..(12): X = V or E
  MISC_FEATURE (13)..(13): X = V or I
IIPVYCALLATXXLGLLAYVAF

SEQ ID NO: 20 - protein, 16 aa, Artificial Sequence
  16-AA consensus non-primate, mammalian p75 binding domain
  MISC_FEATURE (4)..(4): X = H, Q, R, or Y
LAGXLGYQAEAVETMA

SEQ ID NO: 21 - protein, 18 aa, Artificial Sequence
  18-AA consensus non-primate, mammalian p75 binding domain
  MISC_FEATURE (4)..(4): X = H, Q, R, or Y
  MISC_FEATURE (17)..(17): X = C or R
  MISC_FEATURE (18)..(18): X = D or S
LAGXLGYQAEAVETMAXX

SEQ ID NO: 22 - protein, 17 aa, Artificial Sequence
  Non-primate (including X. laevis) consensus p75 binding domain
  MISC_FEATURE (3)..(3): X = G or S
  MISC_FEATURE (4)..(4): X = H, Q, R, or Y
  MISC_FEATURE (8)..(8): X = Q or E
  MISC_FEATURE (9)..(9): X = A or E
  MISC_FEATURE (11)..(11): X = A or T
  MISC_FEATURE (12)..(12): X = V or I
  MISC_FEATURE (13)..(13): X = E or D
  MISC_FEATURE (15)..(15): X = M or F
  MISC_FEATURE (16)..(16): X = A or G
  MISC_FEATURE (17)..(17): X = C or R
LAXXLGYXXEXXXTXXX

SEQ ID NO: 23 - DNA, 1284 bp, Homo sapiens
  CDS (1)..(1284)
atgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtggaacagctgcaagcagaacaagcaaggagccaacagccggccagtgaaccagacgcccccaccagagggagaaaaactccacagcgacagtggcatctccgtggacagccagagcctgcatgaccagcagccccacacgcagacagcctcgggccaggccctcaagggtgacggaggcctctacagcagcctgcccccagccaagcgggaggaggtggagaagcttctcaacggctctgcgggggacacctggcggcacctggcgggcgagctgggctaccagcccgagcacatagactcctttacccatgaggcctgccccgttcgcgccctgcttgcaagctgggccacccaggacagcgccacactggacgccctcctggccgccctgcgccgcatccagcgagccgacctcgtggagagtctgtgcagtgagtccactgccacatccccggtgtga

SEQ ID NO: 24 - protein, 427 aa, Homo sapiens
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSCKQNKQGANSRPVNQTPPPEGEKLHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV

SEQ ID NO: 25 - DNA, 1284 bp, Mus musculus
  CDS (1)..(1284)
atgaggagggcaggtgctgcctgcagcgccatggaccggctgcgcctgctgctgctgctgctgctgcttctaggggtgtcctttggaggtgccaaggagacatgttccacaggcatgtacacccacagtggagagtgctgcaaagcctgcaacctgggcgaaggtgtggcccagccttgcggagccaaccagaccgtgtgtgaaccctgcctggacagtgttacgttctctgacgtggtaagcgccaccgagccgtgtaagccgtgcaccgagtgcctgggcctgcagagtatgtccgctccctgtgtggaggcagacgatgccgtgtgccgatgctcctatggctactaccaggacgaggagactggccgctgcgaggcttgcagcgtgtgcggggtgggctcaggactcgtgttctcctgccaggacaaacagaacacagtgtgtgaagagtgcccagagggcacatactcagatgaagccaaccacgtggacccgtgcctaccctgcacggtgtgcgaggacactgagcgccagttacgcgagtgcacgccctgggctgacgccgaatgcgaggagatccctggccgatggatcacaaggtctacgcccccggagggctctgacgtcacaacacccagcacccaggagccggaggcacctccagagcgagacctcgtagccagcacagtggccgatacagtgaccactgtgatgggcagctcccagcctgtagtgacccgaggcaccgctgacaacctcattcctgtctattgctccatcttggctgctgtggttgtgggccttgtggcctatattgctttcaagagatggaacagctgcaagcaaaataaacaaggagccaacagccggccggtgaaccagacacccccaccagagggagagaaactgcacagcgacagcggcatctctgtggacagccagagcctgcacgaccagcagacccacacacagactgcctcaggccaggccctcaagggtgatggcaacctctacagtagcctgcccctgaccaagcgtgaggaggtcgagaagctgctcaatggtgacacctggcgacatctggcaggcgagctgggctaccagccggagcatatagactcctttacccacgaggcctgcccagtccgagccctgctggccagctggggtgcccaggacagcgcgacgctcgatgcccttttagccgccctgcgacgcatccagagagctgacattgtggagagcctgtgcagcgagtccactgccacatccccagtgtga

SEQ ID NO: 26 - protein, 427 aa, Mus musculus
MRRAGAACSAMDRLRLLLLLLLLLGVSFGGAKETCSTGMYTHSGECCKACNLGEGVAQPCGANQTVCEPCLDSVTFSDVVSATEPCKPCTECLGLQSMSAPCVEADDAVCRCSYGYYQDEETGRCEACSVCGVGSGLVFSCQDKQNTVCEECPEGTYSDEANHVDPCLPCTVCEDTERQLRECTPWADAECEEIPGRWITRSTPPEGSDVTTPSTQEPEAPPERDLVASTVADTVTTVMGSSQPVVTRGTADNLIPVYCSILAAVVVGLVAYIAFKRWNSCKQNKQGANSRPVNQTPPPEGEKLHSDSGISVDSQSLHDQQTHTQTASGQALKGDGNLYSSLPLTKREEVEKLLNGDTWRHLAGELGYQPEHIDSFTHEACPVRALLASWGAQDSATLDALLAALRRIQRADIVESLCSESTATSPV

SEQ ID NO: 27 - DNA, 645 bp, Canis familiaris
  CDS (1)..(645)
aagacacagaggatgcaggacagggatggggaaggggtgtgggttgggggagctctggctcccaatacctcctctccattcccccctgagcctccaggggcctcaggcaacattatccccgtctactgtgccctcttggccaccgtggtcctcggcctgctcgcctacgtggccttcaagtgctggcgctcacataaacaaagacagcagctggccaaagctcggactgcagagctgggggacctcgacagggaccagaggcatggtgacagcattgtcttcctggacaatcctagcggtcgggagccctgtgcccccagccaggggtcacatcctgaacttggctgccggctttaccttcacctccctcggccgcagcaagaggaagtggagcggctcctggaagcatcgggcgaagctgacaagggctggcagggcctagcaggccgcctgggctacccggctgatgctgtggaggtcatagcccggggccagctgccagcccacaccctgcttagggactgggctgtcaaagaaggcagtggtgccaccctgagggtgctagaggacaccctggctgccatgggccgtgaagatgtgctccgggtcctgggtcccccggctgagggctgctctgtggtgtga

SEQ ID NO: 28 - protein, 214 aa, Canis familiaris
KTQRMQDRDGEGVWVGGALAPNTSSPFPPEPPGASGNIIPVYCALLATVVLGLLAYVAFKCWRSHKQRQQLAKARTAELGDLDRDQRHGDSIVFLDNPSGREPCAPSQGSHPELGCRLYLHLPRPQQEEVERLLEASGEADKGWQGLAGRLGYPADAVEVIARGQLPAHTLLRDWAVKEGSGATLRVLEDTLAAMGREDVLRVLGPPAEGCSVV

(19) United States
(12) Patent Application Publication
(10) Pub. No.: US 2007/0004628 A1
(43) Pub. Date: Jan. 4, 2007

(54) Compositions and methods for nerve regeneration

(75) Inventors: Tsung-Chang Sung, San Diego, CA (US); Kuo-Fen Lee, Del Mar, CA (US)

Correspondence Address: KLARQUIST SPARKMAN, LLP, 121 S.W. SALMON STREET, SUITE 1600, PORTLAND, OR, 97204, US

(73) Assignee: The Salk Institute for Biological Studies

(21) Appl. No.: 11/473,609

(22) Filed: Jun. 22, 2006

Related U.S. Application Data
(60) Provisional application No. 60/693,571, filed on Jun. 23, 2005.

(51) Int. Cl. A61K 48/00; A61K 38/17

(52) U.S. Cl. 514/12; 514/44

(57) ABSTRACT

This disclosure demonstrates that p45 promotes rapid regeneration of CNS nerves and axons (for example, in the corticospinal tract and/or raphespinal tract) and locomotor functional recovery following complete spinal cord transection in an adult subject. This remarkable discovery as well as disclosed mapping of p45 functional regions enables, for instance, compositions and methods (e.g., in vivo and in vitro methods), involving p45 proteins, fragments and variants, for promoting nerve growth and/or regeneration, and methods for identifying agents having potential nerve-regenerating activity.

### REFERENCE TO RELATED APPLICATIONS

This application claims the benefit of U.S. Provisional Application No. 60/693,571, filed Jun. 23, 2005, which application is incorporated herein in its entirety.

### STATEMENT OF GOVERNMENT SUPPORT

This invention was made with United States government support pursuant to grant number T32 AG00216 from the National Institutes of Health; the United States government has certain rights in the invention.

### FIELD OF THE DISCLOSURE

This disclosure concerns compositions and methods for promoting nerve growth and/or regeneration, and methods of identifying agents with potential for promoting nerve growth and/or regeneration.

### BACKGROUND

The mammalian nervous system, especially the central nervous system (CNS), naturally has a limited capability to regenerate upon injury. This limited regenerative ability can be attributed to several factors. For example, injury to CNS axons in an adult elicits detrimental inflammatory responses, which are followed by secondary degeneration of the nervous tissues. In addition, regeneration of injured axons is believed to be impeded by the presence or up-regulation of various nerve outgrowth inhibitors, including myelin-associated inhibitors, repulsive axon-guidance molecules, and glial scar-derived chondroitin sulfate proteoglycans, and the absence or down-regulation of factors that promote nerve outgrowth and cell survival, including neurotrophic factors (Schwab and Bartholdi, Physiol. Rev., 76:319, 1996; Filbin, Nat. Rev. Neurosci., 4:703, 2003; Silver and Miller, Nat. Rev. Neurosci., 5:146, 2004).

Compositions and methods that suppress inhibitory mechanisms and/or enhance neurotrophic mechanisms following CNS injury are needed to overcome, at least in part, the limited ability of the CNS to recover from injury.

Known inhibitors of CNS axon regeneration include, for instance, chondroitin sulphate proteoglycans (CSPGs), ephrin-B3, and Nogo. CSPGs are components of the extracellular matrix (ECM) and are naturally occurring throughout the body. During development, CSPGs are thought to play a vital role by forming boundaries that guide migrating neuronal cells to appropriate destinations. Following a head and/or spinal cord injury, the levels of CSPGs are substantially increased at glial scars, which is thought to contribute to failures of nerve cell regeneration and axonal growth (Jones et al., Exp. Neurol., 182:399-411, 2003; Jones et al., J. Neurosci., 23:9276-9288, 2003; Lemons et al., Exp. Neurol., 160:51-65, 1999; Morgenstern et al., Prog. Brain Res., 137:313-332, 2002; Properzi et al., Biochem. Soc. Trans., 31:335-336, 2003; Stichel et al., Brain Res., 704:263-274, 1995; Zuo et al., J. Neurobiol., 34:41-54, 1998; Jones et al., J. Neurosci., 22:2792-2803, 2002; Levine, J. Neurosci., 14:4716-4730, 1994). Consistently, degradation of CSPGs by chondroitinase ABC has been found to promote transplant-mediated axonal remodeling and functional recovery after spinal cord injury in adult rats (Bradbury et al., Nature, 416:636-640, 2002; Kim et al., J. Comp. Neurol., 497: 182-198, 2006).

Ephrin-B3 (EFNB3) is a 340-amino acid, transmembrane protein belonging to the class of ephrin-B (EFNB) ligands, which bind Eph-family receptor protein tyrosine kinases (such as EphA4). EFNB3 shares 38% and 40% identity with related Eph-receptor ligands, EFNB1 and EFNB2, respectively. The N-terminal halves of EFNB1, EFNB2, and EFNB3 are especially conserved, which suggest that these proteins can bind the same subclass of Eph receptor (Tang et al., Genomics, 41:17-24, 1997). Receptors in the Eph subfamily and their ligands have been implicated in mediating developmental events in the nervous system (Kullander et al., Genes Dev., 15:877-888, 2001; Yokoyama et al., Neuron, 29:85-97, 2001). Central pattern generators (which provide neural controls that underlie locomotion) in isolated spinal cords from mice lacking EFNB3 lose the pattern of left-right limb alternation and, instead, exhibit synchrony (Kullander et al., Science, 299: 1889-1892, 2003).

EFNB3 is expressed in postnatal myelinating oligodendrocytes in the mouse spinal cord. EFNB3-EphA4 signaling is believed to play a role in the inhibitory activity of CNS myelin preparations. EFNB3 prevents neurite outgrowth in the primary postnatal EphA4-positive neurons (Benson et al., Proc. Natl. Acad. Sci. USA, 102:10694-10699, 2005). Following spinal cord injury, EphA4 accumulates in proximal axon stumps and EphA4 ligands, EFNB2 and EFNB3, are markedly up-regulated in astrocytes in the glial scar. These events are thought to lead to retraction of corticospinal axons and inhibition of their regeneration (Fabes et al., Eur. J. Neurosci., 23:1721-1730, 2006).

Nogo interacts with its receptor (NgR) to prevent nerve outgrowth. Members of the tumor necrosis factor receptor (TNFR) superfamily have been shown to be involved in NgR-mediated inhibition of nerve regeneration. For example, the neurotrophin receptor p75 (referred to herein as “p75”; also known in the art as p75^{NTR}) and another TNFR, called Troy or Taj, are each believed to independently act as a co-receptor in the Nogo receptor (NgR) complex to transduce nerve growth inhibition signal induced by three myelin-associated inhibitors in cultures, Nogo A, myelin-associated glycoprotein (MAG) and oliodendrocyte-myelin glycoprotein (OMgp) (Wang et al., Nature, 420:74, 2002; Wong et al., Nat. Neurosci., 5:1302, 2002; Dubreuil et al., J. Cell. Biol., 162:233, 2003; Mi et al., Nat. Neurosci., 7:221, 2004; Shao et al., Neuron, 45(3):353-359, 2005; Park et al., Neuron, 45(3):345-351, 2005).

Rho is a small GTP-binding protein that has been suggested to be the central integrator of myelin-derived growth inhibitory signals (McKerracher and Winton, Neuron, 36:345-348, 2002). In the absence of myelin-associated inhibitors (such as MAG or Nogo-A), nerve growth and regeneration are believed to occur as a result of Rho-GDI-induced suppression of Rho activity. In one non-limiting mechanism, myelin-associated inhibitors (such as MAG and Nogo-A) bind to NgR, which, in turn, binds to and activates p75. Activated p75 sequesters Rho-GDI away from Rho, allowing Rho to become activated through the exchange of GDP for GTP. The activated GTP-bound Rho then interacts with signaling proteins such as Rho kinase (ROCK) to suppress axonal growth and regeneration (reviewed in Kaplan and Miller, Nat. Neurosci., 6:435-436, 2003). Accordingly, certain agents that affect p75 may suppress the inhibitory effect of the NgR-mediated pathway on nerve regeneration.

Recently, a 45 kd transmembrane (TM) glycoprotein protein (referred to herein as “p45”) that bears a high degree of homology to the p75 protein was identified. p45 is also known in the art as p75-like apoptosis inducing death domain protein (PLAIDD) (Frankowski et al., Neuromolecular Med., 1:153, 2002), neurotrophin receptor-like death domain protein (NRADD) (Wang et al., Cell Death Differ., 10:580, 2003) or neurotrophin receptor homologue 2 (NRH2) (Kanning et al., J. Neurosci. 23:5425, 2003). p45 and p75 have a high degree of amino acid similarity in their TM (˜94%) and intracellular domain (ICD) (˜50%), including the death domain (DD). However, the p45 extracellular domain (ECD) is short and divergent in sequence as compared to p75 ECD. In particular, the p45 ECD lacks a neurotrophin binding domain. p45 and p75 are expressed in both the peripheral nervous system (PNS) and CNS during development (Kanning et al., J. Neurosci. 23:5425, 2003). In the adult, p45 and p75 are maintained at high levels in the PNS, while expression of both proteins decreases in the CNS (see, e.g., FIG. 1a).

New compositions and methods for promoting nerve growth and/or regeneration, particularly in the CNS, are needed.

### SUMMARY OF THE DISCLOSURE

This disclosure demonstrates that p45 promotes rapid regeneration of nerves of the central nervous system for example, in the corticospinal tract and/or raphespinal tract) and locomotor functional recovery following complete spinal cord transection in an adult subject. This remarkable discovery as well as disclosed mapping of p45 functional regions enables compositions and methods (e.g., in vivo and in vitro methods) involving p45 proteins, fragments and variants, for promoting nerve growth and/or regeneration.

It has also been discovered that p45 forms a protein-protein interaction with p75, FADD, syntaxin binding protein (a.k.a., STXbp1, Munc18-1, or p67), OFD1 (an oral-facial-digital syndrome 1-related protein), mitochondrial membrane proteins (including the 24-kD subunit of mitochondrial NADH:ubiquinone oxidoreductase (Ndufv2), the beta subunit of mitochondrial ATP synthase (Atp5b), dihydrolipoamide S-acetyltransferase (DLAT), and succinate dehydrogenase (Sdha)), and cytoskeleton-regulating proteins (including neural tropomodulin (N-Tmod, Tmod2) and rab GDP dissociation inhibitor-alpha (Rab-GDI)). These newly discovered p45 protein-protein interactions provide a basis for methods of identifying agents that affect one or more of such interactions and thereby have the potential to promote p45-dependent nerve growth and/or regeneration.

In addition, the new appreciation of the nerve-regenerating role of p45 provided by this disclosure further makes possible other methods of identifying potential nerve-regenerating agents; for example, agents that increase the expression of a p45 polypeptide, or agents that increase nerve outgrowth in a cell that expresses more p45 than does another cell, or agents that are specifically recognized by a p45-specific antibody (such as an antibody specific for the amino acids of p45 that are involved in the p45-p75 interaction interface).

The foregoing and other features and advantages will become more apparent from the following detailed description of several embodiments, which proceeds with reference to the accompanying figures.

### BRIEF DESCRIPTION OF THE FIGURES

FIG. 1 includes several panels relating to the expression of p45 and p75 and p45-p75 complex formation in vitro and in vivo. FIG. 1A shows the results of a Western blot of total protein lysates from the brain, spinal cord and dorsal root ganglia (DRG) of post-natal day 0 (P0) and adult animals. The blot was probed with antibodies specific for p45, p75, or the loading control, actin. FIG. 1B shows the results of a Western blot of proteins immunoprecipitated by anti-Myc antibodies from lysates of HEK293 cell co-transfected with full-length p45 (p45-FL) and c-Myc-tagged p75 (c-Myc-p75) expression vectors (+/+), or sham-transfected with vector (pcDNA 3.1) only (−/−). The blot was probed with antibodies specific for p45 or p75 (as shown to the right of the blots). FIG. 1C shows the results of a Western blot of protein that was immunoprecipitated by anti-Flag antibodies from lysates of HEK293 cells (i) co-transfected with expression vectors for a Flag-tagged p75 devoid of the ECD (“Flag-p75-TM-ICD”) and c-Myc-tagged p75 (c-Myc-p75); (ii) co-transfected with full-length p45 (p45-FL) and Flag-p75-TM-ICD expression vectors; (iii) transfected with a Flag-p75-TM-ICD expression vector; or (iv) that were sham-transfected vector (pcDNA 3.1) only. The blot was probed with antibodies specific for p45 or a rabbit polyclonal antibody specific for the intracellular domain of p75, the latter of which will recognize both c-Myc-p75 and Flag-p75-TM-ICD. FIG. 1D is a three-dimensional model of the amino acids 130-228 of p45 ICD obtained from the SWISS-MODEL protein server using the structure of p75 ICD as a template and the secondary structure information from NMR data. The residues most perturbed in the titration experiments and, hence, responsible for the interaction with p75 ICD are labeled by residue number in the model. FIG. 1E shows the results of a Western blot of p75 protein immunoprecipitated (IP) from lysates of post-natal day 7 (P7) cerebellums by anti-p75ECD or anti-p45ECD antibodies or control (CTL) antibodies. Total (T) proteins were used as a control.

FIG. 2 includes several panels demonstrating that p45 interferes with the p75-NgR interaction and signaling. FIG. 2A shows the results of Western blots of proteins immunoprecipitated with antibodies specific for the p75 ICD (α-p75-ICD Ab) from lysates of HEK293 cells (i) co-transfected with vectors expressing p75 and Flag-tagged human NogoR (Flag-hNgR), (ii) co-transfected with vectors expressing p75, Flag-hNgR, and p45, or (iii) that were not transfected. The blot was probed with antibodies specific for the Flag tag or p75. FIG. 2B (top) shows the results of a Western blot of proteins immunoprecipitated by anti-Flag antibody from lysates of HEK293 cells co-transfected with fixed amounts of p75 and Flag-hNgR expression vectors and increasing amounts of a p45 expression vector. The blot was probed with antibodies specific for the Flag tag, p75, or both. FIG. 2B (bottom) shows by Western blot the p45, p75 or Flag-hNgR expression in total lysates of HEK293 cells transfected as described for FIG. 2B (top). FIG. 2C (top) shows the results of a Western blot of proteins immunoprecipitated by anti-Flag antibody from lysates of HEK293 cells co-transfected with expression vectors for the indicated p45 deletion mutant, p75 and Flag-hNgR or mock-transfected cells. The blots were probes with anti-p75 or anti-Flag antibodies. FIG. 2C (middle) shows by Western blot the p75 or Flag-hNgR expression in total lysates of HEK293 cells transfected as described for FIG. 2C (top). FIG. 2C (bottom) shows schematic representations of the p45 deletion mutants used in the transfection experiments and the relative ability of each to block the p75-hNGR interaction. FIG. 2D shows the results of cerebellar granule neurons (CGNs) transfected with p45 RNA and treated with MAG-Fc and subjected to a RhoA activity assay. Over-expression of p45 inhibited MAG-Fc-induced RhoA activation. FIG. 2E is a schematic representation of a non-limiting mechanism by which p45 can block p75-NgR interaction and inhibitory signaling; thereby promoting neurite outgrowth.

FIG. 3 includes several panels demonstrating that p45 over-expression reduces growth inhibition from inhibitory substrates. FIG. 3A shows increased p45 protein levels in P7-P9 CGNs following transfection p45 RNA. FIG. 3B show CGNs seeded on glass coverslips coated with inhibitory substrates, grown for 14-18 hours and immunofluorescently stained with TuJ1 and anti-p45 antibodies. Scale bar=50 μm. FIG. 3C shows a quantitative analysis of neurite length from the outgrowth assay using Nogo66-GST, myelin or HEK293 cells expressing MAG as inhibitory substrates. Data is shown as mean±SEM. (*, p<0.001). FIG. 3D shows a quantitative analysis of neurite length in wild-type and p75−/− CGNs transfected with p45 RNA and plated on Nogo66-coated dishes. p75-deficient CGNs displayed decreased inhibition of neurite outgrowth as compared to control neurons. Over-expression of p45 did not further increase neurite outgrowth.

FIG. 4 includes several panels that demonstrate rapid functional recovery of Thy1-p45 mice following a complete spinal cord (SC) transection. Brain and spinal cord sections of control (A and B) and Thy1-p45 transgenic mice (C and D) were immunostained with anti-p45 antibodies. High levels of p45 expression were detected in numerous neuronal structures, compared with control sections. Ctx (V, VI), cortex layers V&VI; BLA, basolateral amygdala; DG, dentate gyres; Thal, thalamus; fx, fibers of fornix; DH. dosal horn; VH, ventral horn; grf, gracile fasiculus; dcs, dorsal corticospinal; *, spinal projection fibers from dorsal root sensory neurons. FIG. 4E shows representative control and Thy1-p45 mice one week after complete spinal cord transection. The hindlimbs of Thy1-p45 mice were positioned for weight support (arrow), while the hindlimbs of control mice were not. FIG. 4F shows a time course of locomotor activity in control and Thy1-p45 mice using the BMS score system. *, p<0.05; **, p<0.01.

FIG. 5 illustrates the regeneration of the RST in Thy1-p45 mice. Parasagittal spinal cord sections from control and Thy1-p45 mice 6-weeks post-SCI were immunostained with anti-GFAP and anti-5-HT antibodies. GFAP immunoreactivity was detected along the edge of lesion sites in both control (A) and Thy1-p45 (D) mice. White boxes in B and E indicated higher magnification views shown in C and F, respectively. 5-HT immunoreactive fibers were detected in the caudal spinal cord of Thy1-p45 mice (E and arrows in F) as compared to controls (B and C). Anatomical orientation of the spinal cord: V, ventral; D, dorsal; R, rostral; C, caudal. Scale bar=500 μm.

FIG. 6 includes several panels illustrating the regeneration of the CST in Thy1-p45 mice. The CST was anterogradely labeled with biotin-dextran amine (BDA). FIGS. 6A, 6B, and 6E show digital representations of montages of dark field photographs from parasagittal spinal cord sections of control (A) and Thy1-p45 (B, E) mice 6-weeks post-spinal cord transection. White boxes in B and E indicated higher magnification of digital representations of the bright-field photographs shown in C, D (from B, left to right) and F (from E). Arrows indicated regenerating fibers. FIGS. 6G and 6H are line drawings created by superimposing multiple images of serial parasagittal sections of DBA-labeled fibers. Long-distance regeneration of CST fibers was observed caudal to the injury site in Thy1-p45 mice (H), but not in controls (G). Scale bars in A, B, E, G, and H=500 μm; in C, D, and F=100 μm.

FIG. 7 includes several panel demonstrating p45 and p75 complex formation and identification of p45ICD-p75ICD protein interface by NMR. FIG. 7A shows the perturbation of the ¹H and ¹⁵N backbone chemical shift of p45ICD upon stepwise titration with p75ICD. A [¹⁵N, ¹H]-TROSY spectrum of p45ICD (concentration 0.5 mM) was measured after addition of unlabeled p75ICD (p45:p75 ratio, 1:0, 1:0.5, 1:1, 1:2, and 1:4). The bar plot represents the normalized change of the chemical shifts of p45ICD residues at the 1:4 ratio, using the equation N=25[Δ(δ(¹H))²+Δ(δ(¹N)/5)²]^{0.5}, where δ(¹H) and δ(¹⁵N) are the chemical shift in parts per million (ppm) along the ω₁(¹⁵N) and ω₂(¹H) dimensions, respectively (Cheever, et al. Nat. Cell Biol., 3:613-618, 2001). Perturbations larger than 1.0 are labeled. FIG. 7B shows the superposition of the [¹⁵N, ¹H]-TROSY spectrum of ¹³C, 15N-labeled p45ICD in absence (black), and in presence of a four-fold excess of p75ICD (red). Only cross-peaks of C177, T174 and A176 are shown. An arrow indicates the shifting of the ¹⁵N and/or ¹H resonances by complex formation. FIG. 7C shows cross sections, along the ω²(¹H) dimension, for the residues C177, T174 and A176, showing the decrease of the intensity of the peak of free p45ICD and the formation of a new peak assigned to p45ICD in complex with p75. The protein ratio used is labeled on the left. The appearance of both the free p45ICD and the complex p45:p75 peaks in the complex spectra indicate a relatively low binding affinity.

FIGS. 8 and 9 show CLUSTALW alignments (default settings) of the indicated p45 amino acid sequences. In each Figure, the 5′-most shaded region is the transmembrane (TM) domain, the middle shaded region is the p75-binding domain, and the 3′-most shaded region is the PDZ domain binding site. Similarly, in each Figure, the death domain includes the boxed residues and all of the residues between them, and the Chopper domain runs from M. musculus residue 75 (and corresponding residues in the aligned sequences) to M. musculus residue 101 (and corresponding residues in the aligned sequences). A consensus TMD (SEQ ID NO: 19) and consensus p75-binding site (SEQ ID NO: 21) are provided in FIG. 8A.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NO: 1 shows a Mus musculus p45 nucleic acid sequence.

SEQ ID NO: 2 shows a Mus musculus p45 amino acid sequence (encoded by SEQ ID NO: 1).

SEQ ID NOs: 3, 5, and 7 show non-primate, mammalian p45 nucleic acid sequences from Rattus norvegicus, Bos taurus, or Sus scrofa, respectively.

SEQ ID NOs: 4, 6, and 8 show non-primate, mammalian p45 amino sequences encoded by SEQ ID NOs: 3, 5, or 7, respectively.

SEQ ID NO: 9 shows a Xenopus laevis p45 nucleic acid sequence.

SEQ ID NO: 10 shows a Xenopus laevis p45 amino acid sequence (encoded by SEQ ID NO: 9).

SEQ ID NOs: 11, 13, and 15 show primate p45 nucleic acid sequences from Homo sapiens, Pan troglodytes (chimpanzee), or Macaca mulatta (macaque), respectively.

SEQ ID NOs: 12, 14, and 16 show primate p45 amino acid sequences encoded by SEQ ID NOs: 11, 13, or 15, respectively.

SEQ ID NO: 17 shows a Rattus norvegicus p75 nucleic acid sequence.

SEQ ID NO: 18 shows a Rattus norvegicus p75 amino acid sequence (encoded by SEQ ID NO: 17).

SEQ ID NO: 19 shows a consensus amino acid sequence for non-primate, mammalian p45 TM domain.

SEQ ID NO: 20 shows a 16-amino acid consensus sequence for a p75 binding domain of non-primate, mammalian p45.

SEQ ID NO: 21 shows an 18-amino acid consensus sequence for a p75 binding domain of non-primate, mammalian p45.

SEQ ID NO: 22 shows a consensus amino acid sequence for a p75 binding domain of non-primate p45.

SEQ ID NO: 23 shows a Homo sapiens p75 nucleic acid sequence.

SEQ ID NO: 24 shows a Homo sapiens p75 amino acid sequence (encoded by SEQ ID NO: 23).

SEQ ID NO: 25 shows a Mus musculus p75 nucleic acid sequence.

SEQ ID NO: 26 shows a Mus musculus p75 amino acid sequence (encoded by SEQ ID NO: 25).

SEQ ID NO: 27 shows an expected Canis familiaris p45 nucleic acid sequence.

SEQ ID NO: 28 shows an expected Canis familiaris p45 amino acid sequence (encoded by SEQ ID NO: 27).

Additional nucleic acid and amino acid sequences may be referred to herein by GENBANK™ accession number. Unless otherwise expressly provided, it is understood that the sequences given such GENBANK™ accession numbers are incorporated by reference as they existed and were known as of Jun. 16, 2006.

### DETAILED DESCRIPTION

I. Introduction

Disclosed herein are methods for promoting nerve regeneration in a subject. The methods include administering to the subject a therapeutically effective amount of a p45 polypeptide or a nucleic acid encoding the p45 polypeptide. In some embodiments of the method, a p45 polypeptide is a non-primate p45 polypeptide, and in particular examples, a p45 polypeptide is a non-primate, mammalian p45 polypeptide. In other embodiments, a p45 polypeptide includes an amino acid sequence as set forth in SEQ ID NO: 2, 4, 6, 8, or 10, or residues 161-178 of SEQ ID NO: 2, residues 75-228 of SEQ ID NO: 2, residues 53-228 of SEQ ID NO: 2, or residues 53-221 of SEQ ID NO: 2. In still other method embodiments, a p45 polypeptide includes an amino acid sequence having at least 90% sequence identity to SEQ ID NO: 2, residues 161-178 of SEQ ID NO: 2, residues 75-228 of SEQ ID NO: 2, residues 53-228 of SEQ ID NO: 2, or residues 53-221 of SEQ ID NO: 2; and has nerve-regenerating activity.

In some method embodiments, a subject has a spinal cord injury, and in certain examples, the spinal cord injury is a partial transection, a complete transection, or a crush injury of the spinal cord. In particular examples, the spinal cord injury occurs at thoracic vertebral segment T9 or lower, and in even more particular examples, the therapeutically effective amount is sufficient to cause a detectable improvement in the locomotor function of a treated subject as compared to an untreated subject. In some instances, a treated subject regains at least 50% of its locomotor function as compared to an untreated subject. In other method embodiments, a subject is a mammal, and in some instances, a subject is a human. In certain examples, the nerve regeneration occurs in at least the corticospinal tract or the raphespinal tract of a subject, and in particular examples, the nerve regeneration includes motor neuron axon regeneration.

A p45 polypeptide or a nucleic acid encoding the p45 polypeptide of a disclosed methods can be administered by a variety of methods known to those of skill in the art, including, for example intravenous injection, intradural injection, intracranial injection, intrathecal injection, or epidural injection, or intravenous, intradural, intracranial, intrathecal, or epidural infusion with a transplanted minipump. In some examples, a therapeutically effective amount of a disclosed therapeutic agent (such as, a p45 polypeptide) includes from about 0.1 to about 10 mg/kg body weight. In other examples, a nucleic acid encoding a p45 polypeptide (such as, a p45 functional fragment) is a viral vector, a naked DNA, a liposome-encapsulated DNA, or a capped RNA. In particular method examples, a viral vector is an adeno-associated viral vector or a lentiviral vector.

Also disclosed herein are methods for promoting nerve growth that includes contacting a nerve cell with a growth-promoting amount of a p45 polypeptide or a nucleic acid encoding the p45 polypeptide. In some examples, the nerve cell has sustained damage, and, in particular examples, the damage sustained by the nerve cell includes a transection or crush of an axon of the nerve cell. In even more particular examples, nerve growth includes regeneration of the transected or crushed axon. In some method embodiments, contacting the nerve cell occurs in vitro and/or in vivo. In certain examples, the nerve cell expresses a p75 polypeptide.

Another embodiment described herein is an isolated polypeptide, the amino acid sequence of which consists essentially of: (a) residues 161-178 of SEQ ID NO: 2, residues 75-228 of SEQ ID NO: 2, residues 53-228 of SEQ ID NO: 2, or residues 53-221 of SEQ ID NO: 2; (b) residues 161-178 of SEQ ID NO: 4, residues 75-228 of SEQ ID NO: 4, residues 53-228 of SEQ ID NO: 4, or residues 53-221 of SEQ ID NO: 4; (c) residues 162-179 of SEQ ID NO: 6, residues 75-229 of SEQ ID NO: 6, residues 53-229 of SEQ ID NO: 6, or residues 53-222 of SEQ ID NO: 6; (d) residues 176-193 of SEQ ID NO: 8, residues 89-243 of SEQ ID NO: 8, residues 67-243 of SEQ ID NO: 8, or residues 67-236 of SEQ ID NO: 8; or (e) an amino acid sequence having at least 90% sequence identity to any of the sequences set forth in (a)-(d), wherein the amino acid sequence has p75-binding activity or nerve-regenerating activity. A further embodiment is a nucleic acid molecule encoding any one or more of the foregoing isolated polypeptides.

Further embodiments are pharmaceutical compositions that include a pharmaceutically acceptable carrier and an isolated polypeptide having an amino acid sequence that includes the sequence LAGX₁LGYQAEAVETMA; wherein X₁ is H, Q, R, or Y. In some examples, the amino acid sequence includes residues 161-178 of SEQ ID NO: 2, residues 161-178 of SEQ ID NO: 4, residues 162-179 of SEQ ID NO: 6, or residues 176-193 of SEQ ID NO: 8, and in particular examples, the isolated polypeptide has an amino acid sequence consisting essentially of LAGX₁LGYQAEAVETMA; wherein X₁ is H, Q, R, or Y.

Also disclosed are methods for identifying an agent having potential to promote nerve regeneration. Such methods include contacting with at least one test agent a cell that includes a nucleic acid sequence encoding a p45 polypeptide, or a reporter gene operably linked to a p45 transcription regulatory sequence; and detecting an increase in the expression of the p45 polypeptide or the reporter gene in the cell; thereby identifying the at least one test agent as an agent having potential to promote nerve regeneration. In some examples, the cell is a neuron or a glial cell, and in some instances, the glial cell is an oligodendrocyte, astrocyte, or microglial cell. In certain method embodiments, detecting the increase in the p45 polypeptide expression is by Northern blot, Western blot, RT-PCR, immunohistochemistry, quantitative-PCR, or in situ hybridization. In particular method embodiments, a nucleic acid sequence encoding a p45 polypeptide is a p45 gene in the genome of a test cell. Other exemplary screening methods include contacting each of a plurality of cells with a member of a library of compositions. In particular examples, the library of compositions includes at least about 100 different compositions. In yet other particular examples, the library of compositions includes one or more of natural products, chemical compositions, biochemical compositions, polypeptides, peptides, or antibodies.

Methods of identifying an agent having potential to promote nerve regeneration are also disclosed. Such methods involve providing a first component that includes a p45 polypeptide, providing a second component that includes a p45 specific-binding partner (such as a p75 polypeptide, FADD, Ndufv2, Atp5b, DLAT, Sdha, N-Tmod, Rab-GDI, Munc18-1, or OFD1), contacting the first component and the second component with at least one test agent under conditions that would permit the p45 polypeptide and the p45 specific-binding partner (such as a p75 polypeptide, FADD, Ndufv2, Atp5b, DLAT, Sdha, N-Tmod, Rab-GDI, Munc18-1, or OFD1) to bind to each other in the absence of the test agent, and determining whether the test agent affects (e.g., increases or decreases) the binding of the p45 polypeptide and the p45 specific-binding partner to each other, wherein an effect on the binding of the p45 polypeptide and the p45 specific-binding partner to each other identifies the test agent as an agent having potential to promote nerve regeneration. In some embodiments, the method further includes determining whether an agent having potential to promote nerve regeneration specifically binds to a p75 polypeptide. In some examples, a p45 polypeptide includes at least 15 consecutive amino acids of SEQ ID NO: 2, or at least 15 consecutive amino acids of a polypeptide having 90% sequence identity with SEQ ID NO: 2. In more particular method examples, a p45 polypeptide includes residues 161-178 of SEQ ID NO: 2, residues 161-178 of SEQ ID NO: 4, residues 162-179 of SEQ ID NO: 6, or residues 176-193 of SEQ ID NO: 8. In some instances, a p75 polypeptide includes at least 15 consecutive amino acids of SEQ ID NO: 18, or at least 15 consecutive amino acids of a polypeptide having 90% sequence identity with SEQ ID NO: 18. In other more particular instances, a p75 polypeptide includes residues 360-377 of SEQ ID NO: 18. In certain examples, the effect on the binding of a p45 polypeptide and a p45 specific-binding partner (such as a p75 polypeptide) to each other includes an increase in binding affinity. In particular examples, the first component and the second component independently include a cell, a cellular extract, or an isolated polypeptide. In more particular examples, one or both of a p45 polypeptide or a p45 specific-binding partner (such as a p75 polypeptide, FADD, Ndufv2, Atp5b, DLAT, Sdha, N-Tmod, Rab-GDI, Munc18-1, or OFD1) includes a label, which, in some instances, is a fluorescent label. In still more particular method examples, a p45 polypeptide or a p45 specific-binding partner (such as a p75 polypeptide FADD, Ndufv2, Atp5b, DLAT, Sdha, N-Tmod, Rab-GDI, Munc18-1, or OFD1) is bound to a solid substrate.

Other disclosed methods identify an agent having potential to promote nerve outgrowth. Such methods involve contacting with a test agent a first test system comprising a first cell that expresses first amount of a p45 polypeptide; contacting with the same test agent a second test system comprising a second cell that expresses substantially more of the p45 polypeptide than does the first cell; and detecting in the presence of the test agent an increased p45 function (such as nerve (or neurite) outgrowth, growth cone development, or inhibition of MAG-Fc-induced RhoA) in the second cell as compared to the first cell; wherein detection of increased p45 function (such as nerve (or neurite) outgrowth, growth cone development, or inhibition of MAG-Fc-induced RhoA) in the second cell as compared to the first cell identifies the test agent as an agent having potential to promote nerve outgrowth. In some embodiments, the first cell and the second cell are the same cell type, and in certain examples, the first cell and the second cell are each a nerve cell or are each a glial cell. In some embodiments, the first cell and the second cell are cerebellar granular neurons (CGNs), and in other embodiments, the first cell is a neuron from a control mouse and the second cell is a neuron from a p45-transgenic mouse. In some examples, the second cell expresses at least ten-fold more of the p45 polypeptide than does the first cell, and in particular examples, the second cell is transfected with an expression vector encoding the p45 polypeptide and the expression vector overexpresses the p45 polypeptide in the second cell. In even more particular examples, the first amount of the p45 polypeptide is so small as to be undetectable by Western blot. In still other examples, the first cell and the second cell further express a p75 polypeptide (and/or FADD, Ndufv2, Atp5b, DLAT, Sdha, N-Tmod, Rab-GDI, Munc18-1, or OFD1), and in still more particular examples, the first cell and the second cell express substantially the same amount of a p75 polypeptide.

Methods are also disclosed for identifying an agent having the potential to be a p45 mimetic. Such methods include contacting at least one test agent with an antibody specific for a p45 polypeptide, and a test agent that is specifically bound by the antibody is identified as an agent having potential to be a p45 mimetic. In some embodiments, the method further includes determining whether the agent having potential to be a p45 mimetic can specifically bind a p75 polypeptide. In particular examples, the anti-p45 antibody is specific for residues 161-178 of SEQ ID NO: 2.

II. Abbreviations and Terms

| | |
|---|---|
| Atp5b | beta subunit of mitochondrial ATP synthase (Complex V) |
| BDA | biotin-dextran amine |
| BMS | Basso Mouse Scale |
| CGN | cerebellar granule neurons |
| CNS | central nervous system |
| CST | corticospinal tract |
| DD | death domain |
| DLAT | dihydrolipoamide S-acetyltransferase |
| ECD | extracellular domain |
| EFNB3 | Ephrin-B3 |
| FADD | Fas-associated via death domain protein |
| ICD | intracellular domain |
| MAG | myelin-associated glycoprotein |
| Munc18-1 | syntaxin binding protein |
| Ndufv2 | 24-kD subunit of mitochondrial NADH: ubiquinone |
| | oxidoreductase (Complex I) |
| NgR | Nogo receptor |
| NRADD | neurotrophin receptor like death domain protein |
| NRH2 | neurotrophin receptor homologue 2 |
| N-Tmod | neural tropomodulin |
| OMgp | oliodendrocyte-myelin glycoprotein |
| PLAIDD | p75-like apoptosis inducing death domain |
| PNS | peripheral nervous system |
| Rab-GDI | rab GDP dissociation inhibitor-alpha |
| RST | raphespinal tract |
| Sdha | succinate dehydrogenase |
| TM | transmembrane |
| TNFR | tumor necrosis factor receptor |

In order to facilitate review of the various embodiments of the invention, the following explanations of specific terms are provided:

Affects the binding [of a p45 polypeptide and a p45 binding partner]: To alter or change from one state or condition to another; for example, to strengthen or weaken an interaction between a p45 polypeptide and a p45 binding partner (such as, p75, FADD, Ndufv2, Atp5b, DLAT, Sdha, N-Tmod, Rab-GDI, Munc18-1, or OFD1). In some examples, an interaction may be modified so as to promote interaction between the polypeptides (e.g., p45 and p75) such that the polypeptides interact with each other under conditions that would not normally permit the interaction. In other examples, an interaction may be strengthened so that the polypeptides involved in the interaction (e.g., p45 and p75, or p45 and any one of FADD, Ndufv2, Atp5b, DLAT, Sdha, N-Tmod, Rab-GDI, Munc18-1, or OFD1) interact more strongly than they would under control conditions. In still other examples, an interaction may be modified so as to weaken, inhibit, or substantially eliminate an interaction between the polypeptides such that the polypeptides do not interact with each other under conditions that would normally permit the interaction. In yet other examples, an interaction may be weakened or inhibited so that the polypeptides involved in the interaction interact less avidly than they would under control conditions.

Agent: Any substance (such as an atom, molecule, molecular complex, chemical, peptide, protein, protein complex, nucleic acid, or drug) or any combination of substances that is useful for achieving an end or result; for example, a substance or combination of substances useful for increasing gene expression or modulating a protein activity, or useful for modifying or affecting a protein-protein interactions.

Alpha Helix: A particular helical folding of an amino acid chain in a polypeptide molecules, in which the carbonyl oxygens are hydrogen bonded to amide nitrogen atoms three residues along the chain. In a typical alpha helix, the translation of amino acid residues along the long axis of the helix is 0.15 nm and the rotation per residue is 100°; accordingly, there are 3.6 residues per turn. Side chains of helix-resident amino acids are arranged at the outside of the helix.

Analog, derivative or mimetic: An analog is a molecule that differs in chemical structure from a parent compound, for example a homolog (differing by an increment in the chemical structure, such as a difference in the length of an alkyl chain), a molecular fragment, a structure that differs by one or more functional groups, or a change in ionization. Structural analogs are often found using quantitative structure activity relationships (QSAR), with techniques such as those disclosed in Remington (The Science and Practice of Pharmacology, 19th Edition (1995), chapter 28). A derivative is a biologically active molecule derived from the base structure. A mimetic is a molecule that mimics the activity of another molecule, such as a biologically active molecule. Biologically active molecules can include chemical structures that mimic the biological activities of a compound. A “peptidomimetic” is a compound containing one or more peptidic structural elements that is capable of mimicking or antagonizing the biological action(s) of a natural peptide. In some embodiments, a peptidomimetic is a small protein-like chain (a peptide) that contains both natural and non-natural amino acids. In particular, non-limiting examples, a peptidomimetic mimics an activity of a p45 polypeptide, for example a nerve-regeneration activity, a p75-binding activity, a FADD-binding activity, a Ndufv2-binding activity, an Atp5b-binding activity, a DLAT-binding activity, a Sdha-binding activity, a N-Tmod-binding activity, a Rab-GDI-binding activity, a Munc18-1-binding activity, or an OFD1-binding activity.

Antibody: An intact immunoglobulin or an antigen-binding portion thereof. Antigen-binding portions may be produced by recombinant DNA techniques or by enzymatic or chemical cleavage of intact immunoglobulins. Antigen-binding portions include, inter alia, Fab, Fab′, F(ab′)₂, Fv, dAb (Fd), and complementarity determining region (CDR) fragments, single-chain antibodies (scFv), chimeric antibodies, diabodies and polypeptides (including fusion proteins) that contain at least a portion of an immunoglobulin that is sufficient to confer specific antigen binding to the polypeptide. A Fab fragment is a monovalent fragment consisting of the VL, VH, CL and CH1 domains; an F(ab′)₂ fragment is a bivalent fragment comprising two Fab fragments linked by a disulfide bridge at the hinge region; an Fd fragment consists of the VH and CHI domains; an Fv fragment consists of the VL and VH domains of a single arm of an antibody; and a dAb fragment consists of a VH domain (see, e.g., Ward et al., Nature, 341:544-546, 1989).

A “single-chain antibody” (scFv) is a genetically engineered molecule containing the VH and VL domains of one or more antibody(ies) linked by a suitable polypeptide linker as a genetically fused single chain molecule (see, e.g., Bird et al., Science, 242:423-426, 1988; Huston et al., Proc. Natl. Acad. Sci., 85:5879-5883, 1988). Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, e.g., Holliger et al., Proc. Natl. Acad. Sci., 90:6444-6448, 1993; Poljak et al., Structure, 2:1121-1123, 1994). One or more CDRs may be incorporated into a molecule either covalently or noncovalently to make the resultant molecule an immunoadhesin. An immunoadhesin may incorporate the CDR(s) as part of a larger polypeptide chain, may covalently link the CDR(s) to another polypeptide chain, or may incorporate the CDR(s) noncovalently. The CDRs permit the immunoadhesin to specifically bind to a particular antigen of interest. A chimeric antibody is an antibody that contains one or more regions from one antibody and one or more regions from one or more other antibodies.

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a “bispecific” or “bifunctional” antibody has two different binding sites.

A “neutralizing antibody” or “an inhibitory antibody” is an antibody that inhibits at least one activity of a polypeptide, such as by blocking the binding of the polypeptide to a ligand to which it normally binds, or by disrupting or otherwise interfering with a protein-protein interaction of the polypeptide with a second polypeptide. An “activating antibody” is an antibody that increases an activity of a polypeptide. Binding affinity: A measure of how tightly one agent binds to or interacts with another agent. In some embodiments, binding affinity is the measure of how tightly one polypeptide or protein binds to another polypeptide or protein. Mathematically, binding affinity can be expressed as 1/K_{d}. The higher the affinity (lower the K_{d}), the tighter the interaction.

Chopper domain: A domain first demonstrated in p75 that is both necessary and sufficient for initiating p75-mediated cell death. The domain includes 29-amino acids in the cytoplasmic juxtamembrane region of p75 (corresponding to amino acid residues 279-308 of SEQ ID NO: 18) (Coulson et al., J. Biol. Chem., 275(39):30537-30545, 2000). The Chopper domain initiates cell death only if attached to the plasma membrane. Non-membrane-bound, intracellular Chopper peptides act in a dominant-negative manner, blocking p75-mediated death both in vitro and in vivo.

Corticospinal Tract (CST): A massive collection of axons that projects from the cerebral cortex of the brain to the spinal cord. Also known as the pyramidal tract, the term “corticospinal tract” refers to any of the nerves or axons that run from the sensorimotor areas of the cortex to the motor neurons of the cranial nerve nuclei or the ventral horn of the spinal cord. The CST originates in part from the pyramidal cells in the cortex of each cerebral hemisphere and courses through the internal capsule, then through the medullary pyramids. At this point, approximately 80% of the fibers from each hemisphere decussate in the pyramidal decussation and continue to descend in the lateral white column of the opposite side. The remaining 20% continue down ipsilaterally in the ventro-medial white column to innervate the more medially located motor neurons of the axial and proximal muscles.

The crossed fibers in the lateral white columns include both sensory axons (from post-central gyrus and parietal association areas), and motor axons (from precentral gyrus and prefrontal areas). The sensory axons project into the dorsal horn of the grey matter to effect feedback regulation of the input pathways. The motor axons terminate on motor neurons of the distal muscles either directly or indirectly via interneurons. Lesion of the medial corticospinal tracts in primates results, for instance, in inability to sit upright, walk, or climb.

Gene expression: The process by which the coded information of a nucleic acid transcriptional unit (including, for example, genomic DNA or cDNA) is converted into an operational, non-operational, or structural part of a cell, often including the synthesis of a protein. Gene expression can be influenced by external signals; for instance, exposure of a cell, tissue or subject to an agent that enhances gene expression, such as increasing p45 gene expression. Expression of a gene also may be regulated anywhere in the pathway from DNA to RNA to protein. Regulation of gene expression occurs, for instance, through controls acting on transcription, translation, RNA transport and processing, degradation of intermediary molecules such as mRNA, or through activation, inactivation, compartmentalization or degradation of specific protein molecules after they have been made, or by combinations thereof. Gene expression may be measured at the RNA level or the protein level and by any method known in the art, including, without limitation, Northern blot, RT-PCR, Western blot, or in vitro, in situ, or in vivo protein activity assay(s).

The expression of a nucleic acid may be modulated compared to a control state, such as at a control time (for example, prior to administration of a substance or agent that affects regulation of the nucleic acid under observation) or in a control cell or subject, or as compared to another nucleic acid. Such modulation includes but is not necessarily limited to overexpression, underexpression, or suppression of expression. In addition, it is understood that modulation of nucleic acid expression may be associated with, and in fact may result in, a modulation in the expression of an encoded protein or even a protein that is not encoded by that nucleic acid.

Glial cell: A non-neuronal cell that provides support and nutrition, maintains homeostasis, forms myelin, and/or participates in signal transmission in the nervous system. In the human CNS, glia are estimated to outnumber neurons by as much as 50 to 1. Most glia are derived from the ectodermal tissue of the developing embryo. Glial cells include, but are not limited to microglia, macroglia, astrocytes, oligodendrocytes, radial cells, and ependymal cells in the CNS, and Schwann cells and satellite cells in the PNS.

Microglia are specialized macrophages capable of phagocytosis. Though not technically glia because they are derived from monocytes rather than ectodermal tissue, they are commonly categorized as such because of their supportive role to neurons. Microglia are mobile within the CNS, multiplying following trauma or disease.

Astrocytes are the most abundant type of glial cell, and they have numerous projections that anchor neurons to the blood supply. They regulate the external chemical environment of neurons by removing excess ions, notably potassium, and recycling neurotransmitters released during synaptic transmission. Astrocytes also form much of the blood-brain barrier. Astrocytes also can regulate vasoconstriction and vasodilation by producing substances such as arachidonic acid that generate vasoactive metabolites. In addition, astrocytes form gap junctions with other astrocytes, which permit signaling between the cells.

Oligodendrocytes form an insulating layer around CNS axons referred to as myelin. The myelin sheath allows electrical signals to propagate more efficiently along an axon. Ependymal cells line the cavities of the CNS and beat their cilia to help circulate the cerebrospinal fluid. Radial cells provide a scaffold for the outward migration of cortical cells during development, and are found in the cerebellum and retina in the mature organism. In the cerebellum, these are Bergmann glia, which regulate synaptic plasticity. In the retina, these are Müller cells, which participate in bidirectional communication with neurons.

In the peripheral nervous system, Schwann cells play a role similar to that of oligodendrocytes in the CNS, providing myelination to PNS axons. They also have phagocytotic activity. Satellite cells are small cells that help regulate the PNS chemical environment.

Hybridization: Oligonucleotides and their analogs hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence. For example, an oligonucleotide can be complementary to a p45-encoding mRNA, or a p45-encoding dsDNA.

“Specifically hybridizable” and “specifically complementary” are terms that indicate a sufficient degree of complementarity such that stable and specific binding occurs between the oligonucleotide (or its analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions where specific binding is desired, for example under physiological conditions in the case of in vivo assays or systems. Such binding is referred to as specific hybridization.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ and/or Mg⁺⁺ concentration) of the hybridization buffer will determine the stringency of hybridization, though wash times also influence stringency. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, N.Y., 1989, chapters 9 and 11.

For purposes of the present disclosure, “stringent conditions” encompass conditions under which hybridization will only occur if there is less than 25% mismatch between the hybridization molecule and the target sequence. “Stringent conditions” may be broken down into particular levels of stringency for more precise definition. Thus, as used herein, “moderate stringency” conditions are those under which molecules with more than 25% sequence mismatch will not hybridize; conditions of “medium stringency” are those under which molecules with more than 15% mismatch will not hybridize, and conditions of “high stringency” are those under which sequences with more than 10% mismatch will not hybridize. Conditions of “very high stringency” are those under which sequences with more than 6% mismatch will not hybridize.

In particular embodiments, stringent conditions are hybridization at 65° C. in 6×SSC, 5× Denhardt's solution, 0.5% SDS and 100 μg sheared salmon testes DNA, followed by 15-30 minute sequential washes at 65° C. in 2×SSC, 0.5% SDS, followed by 1×SSC, 0.5% SDS and finally 0.2×SSC, 0.5% SDS.

Isolated: An “isolated” biological component (such as a polynucleotide, polypeptide, or cell) has been purified away from other biological components in a mixed sample (such as a cell or tissue extract). For example, an “isolated” polypeptide or polynucleotide is a polypeptide or polynucleotide that has been separated from the other components of a cell in which the polypeptide or polynucleotide was present (such as an expression host cell for a recombinant polypeptide or polynucleotide).

The term “purified” refers to the removal of one or more extraneous components from a sample. For example, where recombinant polypeptides are expressed in host cells, the polypeptides are purified by, for example, the removal of host cell proteins thereby increasing the percent of recombinant polypeptides in the sample. Similarly, where a recombinant polynucleotide is present in host cells, the polynucleotide is purified by, for example, the removal of host cell polynucleotides thereby increasing the percent of recombinant polynucleotide in the sample. Isolated polypeptides or nucleic acid molecules, typically, comprise at least 50%, at least 60%, at least 70%, at least 80%, at least 90%, at least 95% or even over 99% (w/w or w/v) of a sample.

Polypeptides and nucleic acid molecules are isolated by methods commonly known in the art and as described herein. Purity of polypeptides or nucleic acid molecules may be determined by a number of well-known methods, such as polyacrylamide gel electrophoresis for polypeptides, or agarose gel electrophoresis for nucleic acid molecules.

Nerve regeneration: This term refers to the regrowth of neurites, axons, nerves, or the appearance of axonal growth cones following a neuronal injury, disease, or condition. In some non-limiting embodiments, nerve regeneration is accompanied by an increase in nerve function, for example an increase in nerve transmission. In particular non-limiting examples, nerve regeneration is accompanied by an improvement or increase in locomotor functions, for example, an improvement in locomotor function accompanying “motor neuron axon regeneration.” “Promoting nerve regeneration” refers to increasing the regrowth of neurites, axons, nerves, and/or the appearance of axonal growth cones following a neuronal injury, disease, or condition, or blocking or removing an inhibition of the regrowth of neurites, axons, nerves, and/or the appearance of axonal growth cones following a neuronal injury, disease, or condition. A “nerve-regenerating activity” refers to any activity capable of promoting nerve regeneration. As disclosed herein, one specific, non-limiting example of an agent with nerve-regenerating activity is a p45 polypeptide.

Neuron: Also called a nerve cell, a neuron is typically composed of a cell body containing the nucleus, one or more short branches that receive and integrate signals (dendrites), and one projection that transmits signals (the axon). An axon typically has short branches along its length and at its end. Neurons receive and send signals that control the actions of other cells in the body, such as other nerve cells and muscle cells, and can be broadly categorized as belonging to the central nervous system, which includes the brain and spinal cord, or the peripheral nervous system, which includes that portion of the nervous system consisting of the nerves and ganglia outside the brain and spinal cord.

Nucleic acid molecule: This term refers to a polymeric form of nucleotides, which may include both sense and anti-sense strands of RNA, cDNA, genomic DNA, and synthetic forms and mixed polymers of the above. A nucleotide refers to a ribonucleotide, deoxynucleotide or a modified form of either type of nucleotide. A “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A polynucleotide may include either or both naturally occurring and modified nucleotides linked together by naturally occurring and/or non-naturally occurring nucleotide linkages.

Nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications, such as uncharged linkages (for example, methyl phosphonates, phosphotriesters, phosphoramidates, carbamates, etc.), charged linkages (for example, phosphorothioates, phosphorodithioates, etc.), pendent moieties (for example, polypeptides), intercalators (for example, acridine, psoralen, etc.), chelators, alkylators, and modified linkages (for example, alpha anomeric nucleic acids, etc.). The term “nucleic acid molecule” also includes any topological conformation, including single-stranded, double-stranded, partially duplexed, triplexed, hairpinned, circular and padlocked conformations.

Operably linked: A first nucleic acid sequence is operably linked with a second nucleic acid sequence when the first nucleic acid sequence is in a functional relationship with the second nucleic acid sequence. For instance, a promoter is operably linked to a coding sequence if the promoter affects the transcription or expression of the coding sequence. When recombinantly produced, operably linked nucleic acid sequences are generally contiguous and, where necessary to join two protein-coding regions, in the same reading frame. However, nucleic acids need not be contiguous to be operably linked.

Raphespinal tract (RST): Serotonergic cells of the nucleus raphe magnus in the medulla, which receive some of their input from ascending nociceptive fibers of the anterolateral system, give rise to the descending raphespinal tract (RST). The RST fibers descend in the dorsolateral white column, just outside of the dorsal horn of the grey matter, and synapse on the enkephalinergic cells of the substantia gelatinosa. The pathway is believed to provide feedback inhibition of the pain pathway at the first point of entry into the central nervous system.

Regulatory region (or transcriptional regulatory sequence): A nucleic acid sequence comprising a plurality of cis-acting elements, including, without limitation, enhancers, silencers, promoters, transcription terminators, origins of replication, chromosomal integration sequences, 5′ and 3′ untranslated regions, exons and/or introns, which in combination form a functional unit that regulates the transcription of an operably linked second nucleic acid sequence. Typically, at least the predominant portion, of a regulatory region is found upstream (or 5′) of the transcribable nucleic acid sequence (such as a gene) it regulates. In addition, a regulatory region, often, is contiguous (at least in part) with the transcribable sequence it controls. In a genome, some cis-acting elements regulating a particular transcribable nucleic acid sequence can be tens of kilobases away from the transcriptional start site. A “cis-acting regulatory element” or “cis-acting element” is a regulatory control element that is located on the same nucleic acid molecule as the gene (or other nucleic acid sequence) that it regulates. For example, an enhancer is a cis-acting element with respect to the gene whose transcription is increased by enhancer activation. Similarly, a silencer is a cis-acting element with respect to a gene (or other nucleic acid sequence) whose transcription is decreased by silencer activation.

Reporter gene: A nucleic acid sequence that encodes a typically easily assayed product (e.g. firefly luciferase, chloramphenicol acetyltransferase (CAT) and β-galactosidase). A reporter gene may be operably linked to a regulatory control sequence and transfected into cells. If the regulatory control sequence is transcriptionally active in a particular cell type, the reporter gene product will normally be expressed in such cells and its activity may be measured using techniques known in the art. The activity of a reporter gene product can be used, for example, to assess the transcriptional activity of an operably linked regulatory control sequence.

Sequence identity: The similarity between two nucleic acid sequences or between two amino acid sequences is expressed in terms of the level of sequence identity shared between the sequences. Sequence identity is typically expressed in terms of percentage identity; the higher the percentage, the more similar the two sequences.

Methods for aligning sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. USA 85:2444, 1988; Higgins and Sharp, Gene 73:237-244, 1988; Higgins and Sharp, CABIOS 5:151-153, 1989; Corpet et al., Nucleic Acids Research 16:10881-10890, 1988; Huang, et al., Computer Applications in the Biosciences 8:155-165, 1992; Pearson et al., Methods in Molecular Biology 24:307-331, 1994; Tatiana et al., FEMS Microbiol. Lett., 174:247-250, 1999. Altschul et al. present a detailed consideration of sequence-alignment methods and homology calculations (J. Mol. Biol. 215:403-410, 1990).

The National Center for Biotechnology Information (NCBI) Basic Local Alignment Search Tool (BLASTs, Altschul et al., J. Mol. Biol. 215:403-410, 1990) is available from several sources, including the National Center for Biotechnology Information (NCBI, Bethesda, Md.) and on the Internet, for use in connection with the sequence-analysis programs blastp, blastn, blastx, tblastn and tblastx. A description of how to determine sequence identity using this program is available on the internet under the help section for BLAST™.

For comparisons of amino acid sequences of greater than about 30 amino acids, the “Blast 2 sequences” function of the BLAST™ (Blastp) program is employed using the default BLOSUM62 matrix set to default parameters (cost to open a gap [default=5]; cost to extend a gap [default=2]; penalty for a mismatch [default=−3]; reward for a match [default=1]; expectation value (E) [default=10.0]; word size [default=3]; number of one-line descriptions (V) [default=100]; number of alignments to show (B) [default=100]). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 50%, at least 60%, at least 70%, at least 80%, at least 85%, at least 90%, or at least 95% sequence identity.

For comparisons of nucleic acid sequences, the “Blast 2 sequences” function of the BLAST™ (Blastn) program is employed using the default BLOSUM62 matrix set to default parameters (cost to open a gap [default=11]; cost to extend a gap [default=1]; expectation value (E) [default=10.0]; word size [default=11]; number of one-line descriptions (V) [default=100]; number of alignments to show (B) [default=100]). Nucleic acid sequences with even greater similarity to the reference sequences will show increasing percentage identities when assessed by this method, such as at least 60%, at least 70%, at least 75%, at least 80%, at least 85%, at least 90%, at least 95%, or at least 98% sequence identity.

Specific binding: Specific binding refers to the particular interaction between one binding partner (such as a binding agent) and another binding partner (such as a target). Such interaction is mediated by one or, typically, more noncovalent bonds between the binding partners (or, often, between a specific region or portion of each binding partner). In contrast to non-specific binding sites, specific binding sites are saturable. Accordingly, one exemplary way to characterize specific binding is by a specific binding curve. A specific binding curve shows, for example, the amount of one binding partner (the first binding partner) bound to a fixed amount of the other binding partner as a function of the first binding partner concentration. As the first binding partner concentration increases under these conditions, the amount of the first binding partner bound will saturate. In another contrast to non-specific binding sites, specific binding partners involved in a direct association with each other (e.g., a protein-protein interaction) can be competitively removed (or displaced) from such association (e.g., protein complex) by excess amounts of either specific binding partner. Such competition assays (or displacement assays) are very well known in the art. A “p45 specific-binding partner” is a molecular species (such as a polypeptide, like a p75 polypeptide, FADD, Ndufv2, Atp5b, DLAT, Sdha, N-Tmod, Rab-GDI, Munc18-1, and/or OFD1) that specifically binds to a p45 polypeptide. Therapeutically effective amount: A quantity of a specified agent sufficient to achieve a desired effect in a subject being treated with that agent. For example, this may be the amount of a p45 polypeptide necessary to promote, induce, prompt, or stimulate nerve regeneration in a subject. Ideally, a therapeutically effective amount of an agent is an amount sufficient to promote the therapeutic result (e.g, nerve regeneration) without causing a substantial cytotoxic effect in the subject.

Vector: A nucleic acid molecule capable of transporting a non-vector nucleic acid sequence which has been introduced into the vector. One type of vector is a “plasmid,” which refers to a circular double-stranded DNA into which non-plasmid DNA segments may be ligated. Other vectors include cosmids, bacterial artificial chromosomes (BAC) and yeast artificial chromosomes (YAC). Another type of vector is a viral vector, wherein additional DNA segments may be ligated into all or part of the viral genome. Certain vectors are capable of autonomous replication in a host cell into which they are introduced (for example, vectors having a bacterial origin of replication replicate in bacteria hosts). Other vectors can be integrated into the genome of a host cell upon introduction into the host cell and are replicated along with the host genome. Some vectors contain expression control sequences (such as promoters) and are capable of directing the transcription of an expressible nucleic acid sequence that has been introduced into the vector. Such vectors are referred to as “expression vectors.” A vector can also include one or more selectable marker genes and/or genetic elements known in the art.

Unless otherwise explained, all technical and scientific terms used herein have the same meaning as commonly understood by one of ordinary skill in the art to which the invention belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: A Comprehensive Desk Reference, published by VCH Publishers, Inc., 1995 (ISBN 1-56081-569-8).

The singular terms “a,” “an,” and “the” include plural referents unless context clearly indicates otherwise. “Comprising” means “including.” “Comprising A or B” means “including A or B” or “including A and B.” It is further to be understood that all base sizes or amino acid sizes, and all molecular weight or molecular mass values, given for nucleic acids or polypeptides are approximate, and are provided for description.

All publications, patent applications, patents, and other references mentioned herein are incorporated by reference in their entirety to the extent permitted by applicable law. In case of conflict, the present specification, including explanations of terms, will control.

Suitable methods and materials for the practice or testing embodiments of the invention are described below. However, the provided materials, methods, and examples are illustrative only and are not intended to be limiting. Accordingly, except as otherwise noted, the methods and techniques of the present invention can be performed according to methods and materials similar or equivalent to those described and/or according to conventional methods well known in the art and as described in various general and more specific references that are cited and discussed throughout the present specification (see, e.g., Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; and Harlow and Lane, Antibodies: A Laboratory Manual, Cold Spring Harbor Laboratory Press, 1990 and 1999).

III. Polypeptides and Nucleic Acids

One of the remarkable discoveries disclosed herein is the ability of p45 polypeptides (such as non-primate p45 polypeptides) to promote nerve regrowth following damage to nerves that do not normally regenerate (such as central nervous system neurons). In some embodiments, the recovery is so profound that injured (e.g., paraplegic) subjects actually regain weight supporting and locomotor abilities following usually debilitating spinal cord injury (such as a complete transection, a partial transection, or a crush injury). This striking discovery makes possible a variety of compositions and methods involving p45 polypeptides, which include, without limitation, naturally occurring p45 proteins (e.g., as expressed in a variety of species, such as Mus sp., Rattus sp., Sus sp., Xenopus sp., and Bos sp.), p45 functional fragments, and p45 functional variants, and the nucleic acid sequences that encode such polypeptides.

Representative p45 polypeptides and the nucleic acid sequences encoding such polypeptides are shown in SEQ ID NOs: 2, 4, 6, 8, and 10 (amino acids) and SEQ ID NOs: 1, 3, 5, 7, and 9 (nucleic acids), respectively. Moreover, an expected canine (Canis familiaris) p45 amino acid sequence (based, at least in part, on homology to Mus musculus and other p45 amino acid sequences) and a corresponding nucleic acid sequence are provided in SEQ ID NOs: 28 and 27, respectively.

Exemplary p45 polypeptide fragments having functional activities (e.g., p75 binding activity or nerve-regenerating activity) of the full-length p45 sequences from which the fragments derive are provided in residues 161-178 of SEQ ID NO: 2 or 4 (p75 binding domain), residues 75-228 of SEQ ID NO: 2 or 4 (ICD), residues 53-228 of SEQ ID NO: 2 or 4 (TM/IC domains), residues 53-221 of SEQ ID NO: 2 or 4 (TM/death domains), residues 162-179 of SEQ ID NO: 6 (p75 binding domain), residues 75-229 of SEQ ID NO: 6 (ICD), residues 53-229 of SEQ ID NO: 6 (TM/IC domains), residues 53-222 of SEQ ID NO: 6 (TM/death domains), residues 176-193 of SEQ ID NO: 8 (p75 binding domain), residues 89-243 of SEQ ID NO: 8 (ICD), residues 67-243 of SEQ ID NO: 8 (TM/IC domains), residues 67-236 of SEQ ID NO: 8 (TM/death domains) or as otherwise described herein.

Having provided herein a powerful motivation for which to make p45 polypeptides (such as non-primate, wild-type p45 polypeptides, or functional fragments or functional variants thereof) and p45 nucleic acid sequences, any method known to those of skill in the art may be used to isolate or produce such p45 polypeptides and nucleic acid sequences. For example, p45 polypeptides and nucleic acids may be isolated from sources (e.g., nuclei, cells, tissues, organs, etc.) in which such biological molecules are naturally present. An alternative and advantageous method is to use common molecular biological techniques to produce p45 polypeptides (e.g., non-primate, wild-type p45 polypeptides, or fragments, or variants thereof) from the corresponding nucleic acid sequences. As will be appreciated by the ordinarily skilled artisan, the provision of an amino acid sequence of, or a nucleic acid sequence encoding, a polypeptide of interest is sufficient to enable such artisan to produce either of such biological molecules as well as any desired variants or fragments thereof. Representative p45 amino acid and nucleic acid sequences are provided herein (see, e.g., SEQ ID NOs: 1-10) and others can be mined from publicly available databases using data-mining techniques common in the art.

A. p45 Nucleic Acid Sequences

As any molecular biology textbook teaches, a polypeptide of interest is encoded by its corresponding nucleic acid sequence (e.g., an mRNA or genomic DNA). Accordingly, nucleic acid sequences encoding p45 polypeptides (e.g., non-primate, wild-type p45 polypeptides, or fragments, or variants thereof) are contemplated herein, at least, to make and use p45 polypeptides of the disclosed compositions and methods, and also for use in disclosed compositions and methods.

In one example, in vitro nucleic acid amplification (such as polymerase chain reaction (PCR)) may be utilized as a simple method for producing p45 nucleic acid sequences. PCR is a standard technique, which is described, for instance, in PCR Protocols: A Guide to Methods and Applications (Innis et al., San Diego, Calif.:Academic Press, 1990), or PCR Protocols, Second Edition (Methods in Molecular Biology, Vol. 22, ed. by Bartlett and Stirling, Humana Press, 2003).

A representative technique for producing a p45 nucleic acid molecule by PCR involves preparing a sample containing a target nucleic acid molecule that includes the p45 sequence. For example, DNA or RNA (such as mRNA or total RNA) may serve as a suitable target nucleic acid molecule for PCR reactions. Optionally, the target nucleic acid molecule is extracted from cells by any one of a variety of methods well known to those of ordinary skill in the art (e.g., Sambrook et al., Molecular Cloning: A Laboratory Manual, Cold Spring Harbor Laboratory Press, New York, 1989; Ausubel et al., Current Protocols in Molecular Biology, Greene Publ. Assoc. and Wiley-Intersciences, 1992). p45 is expressed in a variety of cell types; for example, cerebellar granule neurons, dorsal root ganglion neurons, spinal motor neurons, skin, lung, pancreas, thymus, kidney, spleen, and gut. In examples where RNA is the initial target, the RNA is reverse transcribed (using one of a myriad of reverse transcriptases commonly known in the art) to produce a double-stranded template molecule for subsequent amplification. This particular method is known as reverse transcriptase (RT)-PCR. Representative methods and conditions for RT-PCR are described, for example, in Kawasaki et al. (In PCR Protocols, A Guide to Methods and Applications, Innis et al. (eds.), 21-27, Academic Press, Inc., San Diego, Calif., 1990).

The selection of amplification primers will be made according to the portion(s) of the target nucleic acid molecule that is to be amplified. In various embodiments, primers (typically, at least 10 consecutive nucleotides of a p45 nucleic acid sequence) may be chosen to amplify all or part of a p45-encoding sequence. Variations in amplification conditions may be required to accommodate primers and amplicons of differing lengths and composition; such considerations are well known in the art and are discussed for instance in Innis et al. (PCR Protocols, A Guide to Methods and Applications, San Diego, Calif.:Academic Press, 1990). From a provided p45 nucleic acid sequence, one skilled in the art can easily design many different primers that can successfully amplify all or part of a p45-encoding sequence.

This disclosure comprehends p45 nucleotide variants; such as those that encode p45 functional fragments or p45 functional variants. Such nucleotide variants may be naturally occurring or produced using commonly known techniques, including without limitation site-directed mutagenesis or PCR. Standard techniques for DNA mutagenesis are provided, for instance, in Sambrook et al. (Molecular Cloning: A Laboratory Manual, New York:Cold Spring Harbor Laboratory Press, 1989, Ch. 15). In addition, numerous commercially available kits are available to perform DNA mutagenesis (see, for example, QUIKCHANGE™ Site-Directed Mutagenesis Kit (Stratagene), GENETAILOR™ Site-Directed Mutagenesis System (Invitrogen); GPS™-M Mutagenesis System (New England Biolabs, DIVERSIFY™ PCR Random Mutagenesis Kit (BD Biosciences Clontech); Mutation Generation System (MJ Research); EXSITE™ PCR-Based Site-Directed Mutagenesis Kit (Stratagene); GENEMORPH™ PCR Mutagenesis Kit (Stratagene); or LA PCR Mutagenesis Kit (Takara Mirus Bio)).

Variant p45 nucleic acid sequences differ from disclosed or otherwise publicly available sequences by deletion, addition, or substitution of nucleotides, and encode a protein that retains at least one p45 function. Functions of a p45 polypeptide include, without limitation, the ability to promote nerve regeneration (or neurite outgrowth) in vivo or in vitro (for example, regeneration of CNS nerves or the axons of CNS neurons, such as CST motor neurons or RST neurons); specifically bind to p75, FADD, Ndufv2, Atp5b, DLAT, Sdha, N-Tmod, Rab-GDI, Munc18-1, and/or OFD1; enhance TrkA binding of nerve growth factor (NGF); antagonize other members of the TNFR family; promote expression of molecules that prevent the formation of or dissolve glial scar tissue (e.g. tissue plasminogen activators); prevent or delay inflammatory responses; decrease cell death; decrease de-myelination; inhibit CSPG- and/or EFNB3-dependent nerve growth inhibition, and/or increase the secretion of neurotrophic factors (e.g. BDNF, GDNF, NGF, or NT-3). In some embodiments, p45 nucleic acid variants share at least 70%, at least 75%, at least 80%, at least 85%, at least 90%, at least 95%, or at least 98% nucleotide sequence identity with a disclosed (or otherwise known) p45 nucleic acid sequence (including, e.g., SEQ ID NO: 1, 3, 5, 7, or 9). Alternatively, related nucleic acid molecules can have no more than 3, 5, 10, 20, 50, 75, or 100 nucleic acid changes compared to SEQ ID NO: 1, 3, 5, 7, or 9.

An alternative indication that two nucleic acid molecules are closely related (e.g., are variants of one another) is that the two molecules hybridize to each other. In certain embodiments, p45 nucleic acid variants hybridize to a disclosed (or otherwise known) p45 nucleic acid sequence (including, e.g., SEQ ID NO: 1, 3, 5, 7, or 9, or fragments thereof), for example, under low stringency, high stringency, or very high stringency conditions. Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ concentration) of the hybridization buffer will determine the stringency of hybridization, although wash times also influence stringency. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, N.Y., 1989, chapters 9 and 11.

The following are representative hybridization conditions and are not meant to be limiting.

Very High Stringency (detects sequences
that share about 90% sequence identity)

| | |
|---|---|
| Hybridization: | 5x SSC at 65° C. for 16 hours |
| Wash twice: | 2x SSC at room temperature (RT) for 15 minutes each |
| Wash twice: | 0.5x SSC at 65° C. for 20 minutes each |

High Stringency (detects sequences that
share about 80% sequence identity or greater)

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

Low Stringency (detects sequences that
share greater than about 50% sequence identity)

| | |
|---|---|
| Hybridization: | 6x SSC at RT to 55° C. for 16-20 hours |
| Wash at least | 2x-3x SSC at RT to 55° C. for 20-30 minutes each. |
| twice: | |

One of ordinary skill in the art will appreciate that p45-derived oligonucleotides of various lengths are useful for a variety purposes, such as probes, primers, and to encode p45 functional fragments. In some embodiments, an oligonucleotide may comprise at least 15, at least 20, at least 23, at least 25, at least 30, at least 35, at least 40, at least 45, at least 50 or more consecutive nucleotides of p45 nucleotide sequences. In other examples, p45 oligonucleotides (such as those encoding p45 functional fragments) can be at least 100, at least 150, at least 200, at least 250 or at least 300 consecutive nucleic acids of a p45 nucleic acid sequence (such as SEQ ID NO: 1, 3, 5, 7, or 9).

Oligonucleotides (or other p45 nucleic acid fragment) may be obtained from any region of a p45 nucleic acid sequence. By way of example, a p45 nucleic acid sequence (such as SEQ ID NO: 1, 3, 5, 7, or 9) may be apportioned into about halves, thirds or quarters based on sequence length, and the isolated nucleic acid molecules (e.g., oligonucleotides) may be derived from the first or second halves of the molecules, from any of the three thirds, or from any of the four quarters. A p45 nucleic acid sequence also could be divided into smaller regions, e.g. about eighths, sixteenths, twentieths, fiftieths and so forth, with similar effect. One of ordinary skill in the art can readily ascertain from the provided sequences which nucleotide numbers correspond to the described portions of a full-length p45 sequence. For example, the first half of SEQ ID NO: 1 (not including any fractions of a nucleotide) corresponds to nucleotides 1-343 of SEQ ID NO: 1 and so forth.

This disclosure particularly contemplates functional p45 fragments and provides non-limiting examples of the same. p45 nucleic acid sequences encoding certain exemplary p45 functional fragments include, for instance, residues 481-534 of SEQ ID NO: 1 or 3 (p75 binding domain), residues 223-687 of SEQ ID NO: 1 or 3 (ICD), residues 157-687 of SEQ ID NO: 1 or 3 (TM/IC domains), residues 157-663 of SEQ ID NO: 1 or 3 (TM/death domains), residues 484-537 of SEQ ID NO: 5 (p75 binding domain), residues 223-690 of SEQ ID NO: 5 (ICD), residues 157-690 of SEQ ID NO: 5 (TM/IC domains), residues 157-666 of SEQ ID NO: 5 (TM/death domains), residues 526-579 of SEQ ID NO: 7 (p75 binding domain), residues 265-729 of SEQ ID NO: 7 (ICD), residues 99-729 of SEQ ID NO: 7 (TM/IC domains), or residues 99-708 of SEQ ID NO: 7 (TM/death domains). In advantageous embodiments, a p45 functional fragment includes a p45 intracellular (IC) domain and/or transmembrane (TM) domain.

B. p45 Polypeptides

This disclosure further provides compositions and methods involving p45 polypeptides. p45 polypeptides include any disclosed or otherwise known naturally occurring p45 protein (e.g., non-primate p45 homolog) or fragment or variant thereof. In preferred embodiments, a p45 polypeptide is capable of, at least, nerve-regenerating activity, axon outgrowth activity, and/or specifically binding to p75, FADD, Ndufv2, Atp5b, DLAT, Sdha, N-Tmod, Rab-GDI, Munc18-1, and/or OFD1. Representative p45 polypeptides are provided in SEQ ID NOs: 2, 4, 6, 8, or 10, and residues 161-178 of SEQ ID NO: 2 or 4 (p75 binding domain), residues 75-228 of SEQ ID NO: 2 or 4 (ICD), residues 53-228 of SEQ ID NO: 2 or 4 (TM/IC domains), residues 53-221 of SEQ ID NO: 2 or 4 (TM/death domains), residues 162-179 of SEQ ID NO: 6 (p75 binding domain), residues 75-229 of SEQ ID NO: 6 (ICD), residues 53-229 of SEQ ID NO: 6 (TM/IC domains), residues 53-222 of SEQ ID NO: 6 (TM/death domains), residues 176-193 of SEQ ID NO: 8 (p75 binding domain), residues 89-243 of SEQ ID NO: 8 (ICD), residues 67-243 of SEQ ID NO: 8 (TM/IC domains), residues 67-236 of SEQ ID NO: 8 (TM/death domains), or as otherwise described herein. As described in Example 8, genes encoding primate (e.g., human, chimp and macaque) p45 polypeptides have accumulated mutations, which, for example, encode a premature stop signal (see, e.g., SEQ ID NOs: 11, 13, and 15). Thus, naturally occurring primate p45 polypeptides have C-terminal truncations and have lost portions of the transmembrane (TM) and/or intracellular (IC) domains (see, e.g., SEQ ID NOs: 12, 14, and 16). Thus, in some examples, a p45 polypeptide is a non-primate p45 polypeptide (such as a p45 polypeptide from Mus sp., Rattus sp., Sus sp., Xenopus sp., or Bos sp.). In more particular examples, a p45 polypeptide is a non-primate, mammalian p45 polypeptide. In still other examples, a p45 polypeptide is a functional p45 fragment (e.g, a fragment of a non-primate, mammalian p45 protein), which fragment includes (or consists of) a p75-binding domain (or other domain involved in a protein-protein interaction between p45 and another binding partner, such as FADD, Ndufv2, Atp5b, DLAT, Sdha, N-Tmod, Rab-GDI, Munc18-1, and/or OFD1 (such as, an IC domain)), an IC domain, a TM domain and an IC domain, a death domain, a TM domain and a portion of the IC domain that includes the p75-binding domain, or a TM domain and a portion of the IC domain that includes the death domain. In particular examples, a functional p45 fragment includes a p75-binding domain, or a TM domain and an IC domain.

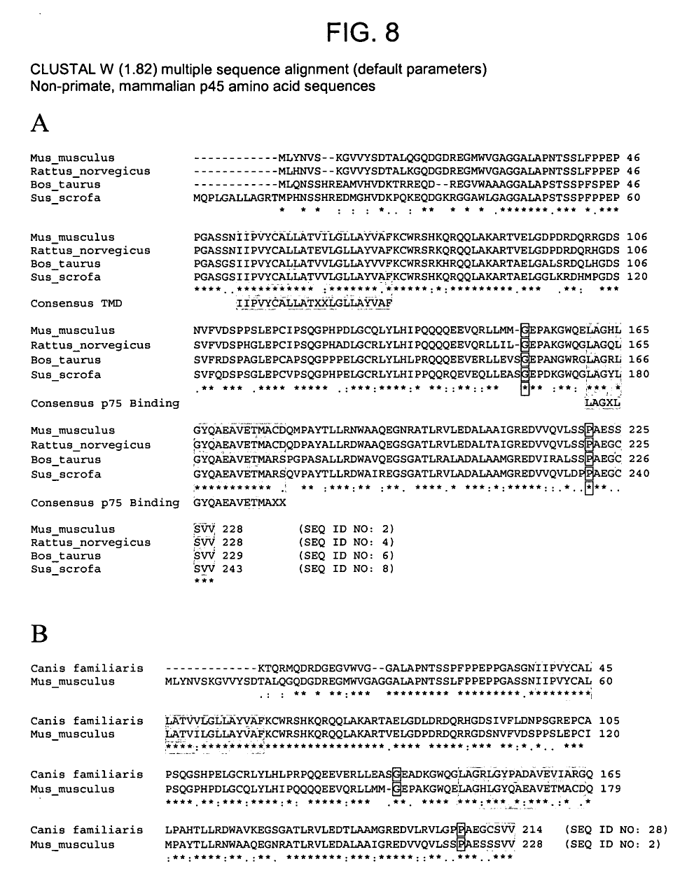

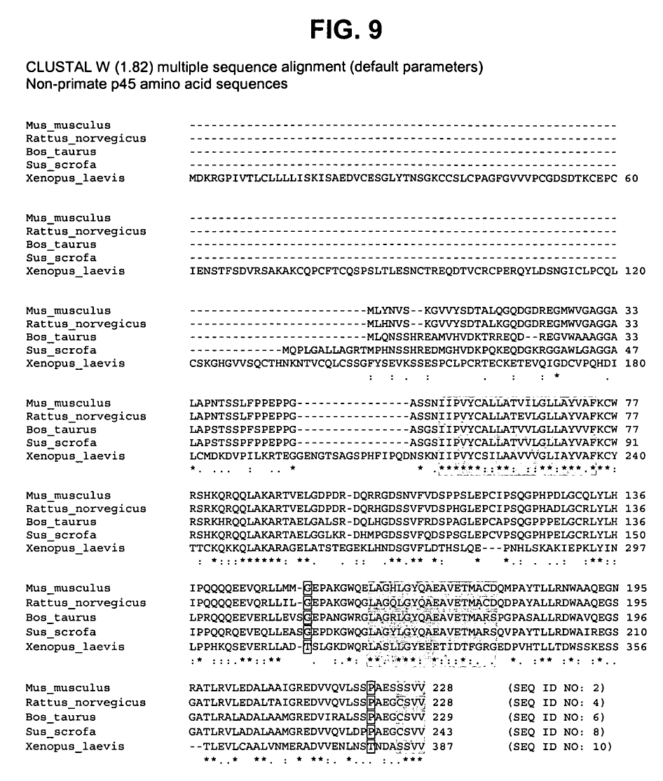

Non-limiting examples of p45 functional domains, which may be expressed as p45 functional fragments, are shown in the following table and in FIGS. 8 and 9:

| | | M. musculus | R. norvegicus* | B. taurus* | S. Scofa* | X. laevis |
|---|---|---|---|---|---|---|
| TM domain | NT | 157-222^{a} | 157-222^{c} | 157-222^{e} | 199-264^{g} | 646-711ⁱ |
| | AA | 53-74^{b} | 53-74^{d} | 53-74^{f} | 67-88^{h} | 216-237^{j} |
| IC domain | NT | 223-687 | 223-687 | 223-690 | 265-729 | 712-1161 |
| | AA | 75-228 | 75-228 | 75-229 | 89-243 | 238-387 |
| Chopper | NT | 223-303 | 223-303 | 223-303 | 265-345 | 712-792 |
| domain | AA | 75-101 | 75-101 | 75-101 | 89-115 | 238-264 |
| p75 binding | NT | 481-534 | 481-534 | 484-537 | 526-579 | 994-1017 |
| domain | AA | 161-178 | 161-178 | 162-179 | 176-193 | 332-339 |
| Death | NT | 454-663 | 454-663 | 457-666 | 499-708 | 934-1140 |
| domain | AA | 152-221 | 152-221 | 153-222 | 167-236 | 312-380 |
| PDZ domain | NT | 673-684 | 673-684 | 676-687 | 718-729 | 1150-1161 |
| binding site | AA | 225-228 | 225-228 | 226-229 | 240-243 | 384-387 |

*Residue numbers are based on alignment with corresponding regions in M. musculus sequences
^{a}residues of SEQ ID NO: 1
^{b}residues of SEQ ID NO: 2
^{c}residues of SEQ ID NO: 3
^{d}residues of SEQ ID NO: 4
^{e}residues of SEQ ID NO: 5
^{f}residues of SEQ ID NO: 6
^{g}residues of SEQ ID NO: 7
^{h}residues of SEQ ID NO: 8
ⁱresidues of SEQ ID NO: 9
^{j}residues of SEQ ID NO: 10

A p45 polypeptide as described in a disclosed composition or method has at least one function of the prototypical p45 polypeptide (SEQ ID NO: 2, as encoded by SEQ ID NO: 1) described in the Examples herein.

Such p45 functions include, without limitation, the ability to promote nerve regeneration (or neurite outgrowth) in vivo or in vitro (for example, regeneration of CNS nerves or axons of CNS neurons, such as CST motor neurons or RST neurons); specifically bind to p75, FADD, Ndufv2, Atp5b, DLAT, Sdha, N-Tmod, Rab-GDI, Munc18-1, and/or OFD1; enhance TrkA binding of nerve growth factor (NGF); antagonize other members of the TNFR family; promote expression of molecules that prevent the formation of or dissolve glial scar tissue (e.g. tissue plasminogen activators); prevent or delay inflammatory responses; decrease cell death; decrease de-myelination; inhibit CSPG- and/or EFNB3-dependent nerve growth inhibition, and/or increase the secretion of neurotrophic factors (e.g. BDNF, GDNF, NGF, or NT-3).

p45 functional variants include proteins that differ in amino acid sequence from disclosed or otherwise known p45 sequences (e.g., naturally occurring, non-primate p45 proteins, such as shown in SEQ ID NO: 2, 4, 6, 8, or 10), but that substantially retain a wild-type function. In some embodiments, p45 functional variants include proteins that share at least 70% amino acid sequence identity with a p45 polypeptide sequence provided herein; for example, some p45 functional variants will share at least 75%, at least 80%, at least 85%, at least 90%, at least 95%, or at least 98% amino acid sequence identity with a disclosed or otherwise known p45 sequences (e.g., naturally occurring, non-primate p45 proteins, such as shown in SEQ ID NO: 2, 4, 6, 8, or 10).

p45 variants can be naturally occurring or produced by any method known in the art for making protein variants. In some embodiments, a p45 variant is produced by manipulation of a disclosed (or otherwise known) p45 nucleotide sequence using standard procedures, including without limitation site-directed mutagenesis or PCR. Techniques for DNA mutagenesis have been described previously herein. Naturally occurring p45 variants can be isolated using any of a myriad of protein purification techniques known in the art (for example, Scopes, Protein Purification: Principles and Practice, 3rd Edition, New York:Springer-Verlag, 1994; Protein Purification Techniques, 2nd Edition, ed. by Simon Roe, New York: Oxford University Press, 2001; Membrane Protein Purification and Crystallization, 2nd Edition, ed. by Hunte et al., San Diego:Academic Press, 2003). A nucleic acid sequence that encodes all or part of a p45 variant can be readily determined simply by applying an appropriate genetic code (such as the eukaryotic genetic code) to the respective portion of the variant's amino acid sequence. The nucleic acid sequence of a variant, then, can be isolated using methods described elsewhere in this specification.

In some embodiments, p45 variants involve the substitution of one or several amino acids for amino acids having similar biochemical properties (so-called conservative substitutions). Conservative amino acid substitutions are likely to have minimal impact on the activity of the resultant protein. Further information about conservative substitutions can be found, for instance, in Ben Bassat et al. (J. Bacteriol., 169:751-757, 1987), O'Regan et al. (Gene, 77:237-251, 1989), Sahin-Toth et al. (Protein Sci., 3:240-247, 1994), Hochuli et al. (Bio/Technology, 6:1321-1325, 1988) and in widely used textbooks of genetics and molecular biology. In some examples, p45 variants can have no more than 3, 5, 10, 15, 20, 25, 30, 40, or 50 conservative amino acid changes compared to SEQ ID NO: 2 (or SEQ ID NO: 4, 6, 8, or 10). The following table shows exemplary conservative amino acid substitutions:

| Original Residue | Conservative Substitutions |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

As described herein, p75-binding activity of p45 can be localized, at least in part, to the ICD or the ICD in combination with the TM and, in particular examples, to p45 polypeptides including any of the consensus sequences, LAGX₁LGYQAEAVETMA[C/R][D/S] (wherein X₁ is H, Q, R, or Y) (SEQ ID NO: 21), LAGX₁LGYQAEAVETMA (wherein X₁ is H, Q, R, or Y) (SEQ ID NO: 20), or LA[G/S]X₁LGY[Q/E][A/E]E[A/T][V/I][E/D]T[M/F][A/G][C/R] (wherein X₁ is H, Q, R, or Y) (SEQ ID NO: 22). Accordingly, in certain embodiments, substitutions of residues outside these regions (e.g., in the extracellular (EC) domain) are likely to have little (or no) substantial effect on protein function. In particular examples, substitution of residues H164, E170, E173 and/or C177 of a p75 binding domain (or corresponding residues in non-mouse p45 homologs, such as human p45) are likely to have little (or no) substantial effect on p45-p75 binding.

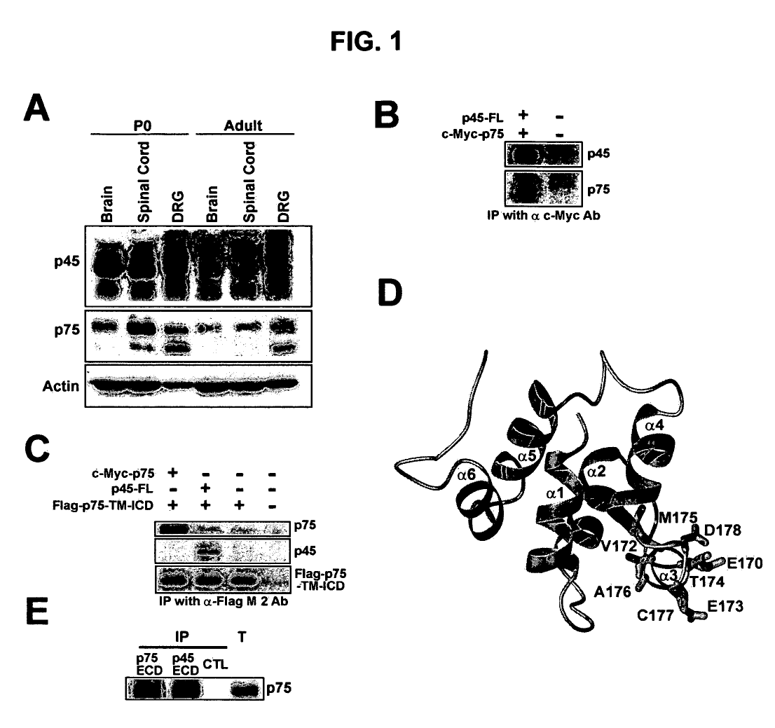

It is expected that mutations that substantially maintain the structure (such as the 3-D structure) of the p45 ICD or p45 binding interface with p75 (see, e.g., FIG. 1) will have little effect on p45 function. The provision of an NMR structure of p45 functional domains (e.g., p75 binding domain) herein permits the design of structurally equivalent, p45 functional variants. As shown, for instance, in Example 2, the amino acid residues of mouse p45 that are involved in p75 binding (which binding has been shown to be involved, at least in part, in the nerve-regenerating activity of p45) are located at the helix3 of p45ICD (A169, E170, V172, E173, T174, M175, A176, C177), with some at helix2 (L161, A162) and the loop between helix3 and helix4 (D178). As known to those of skill in the art, alpha helices (such as helix 3) are destabilized by (i) the substitution of Pro for any helix-resident amino acid, (ii) Asp adjacent to Glu in a helix, or (iii) a cluster of Ile residues (such as 3 or more contiguous Ile residues) in a helix. Accordingly, p45 variants preferably avoid mutations that would destabilize helix 3.

C. p75 Nucleic Acids and Polypeptides

As disclosed for the first time herein, p45 specifically binds to p75. p75 is an essential part of the NgR complex, which complex is centrally involved in a pathway that inhibits nerve regeneration. The formation of a p45-p75 complex is further shown herein to suppress p75-NgR-mediated nerve regeneration. Such discovery makes possible methods of screening for agents that affect the p45-p75 complex, which agents have the potential to also affect nerve regeneration. Accordingly, disclosed methods involve p75 polypeptides (such as naturally occurring p75 proteins, p45-binding fragments thereof, and p45-binding variants thereof) and nucleic acid molecules encoding the same. Representative p75 nucleic acid sequences and the corresponding encoded amino acid sequences are provided in SEQ ID NO: 17 and SEQ ID NO: 18 (rat p75 cDNA and amino acid sequence, respectively), SEQ ID NO: 23 and SEQ ID NO: 24 (human p75 cDNA and amino acid sequence, respectively), and SEQ ID NO: 25 and SEQ ID NO: 26 (mouse p75 cDNA and amino acid sequence, respectively).

p75 nucleic acid sequences, proteins, fragments and variants can be isolated and produced in the same manner as discussed in detail above for p45. Some exemplary p45-binding fragments of p75 include residues 360-377 of SEQ ID NO: 18, residues 362-379 of SEQ ID NO: 24, and residues 362-379 of SEQ ID NO: 26.

D. Additional p45 Binding Partners

Also disclosed herein other p45 specific-binding partners, including FADD, Ndufv2, Atp5b, DLAT, Sdha, N-Tmod, Rab-GDI, Munc18-1, and/or OFD1. p45 is expected to interact with its specific-binding partners via its IC domain, e.g., residues 75-228 of SEQ ID NO: 2, residues 75-228 of SEQ ID NO: 4; residues 75-229 of SEQ ID NO: 6, or residues 89-243 of SEQ ID NO: 8. Accordingly, some disclosed method embodiments involve a p45 specific-binding partner and a p45 IC domain.

FADD is a commonly known adaptor for TNFR and FAS and, as such, is involved in cell-death signaling. Without being bound to theory, it is expected that interfering with FADD-TNFR/FAS interactions (e.g., by FADD binding to p45) prevents or inhibits cell death induced by TNFR and/or FAS. p45-dependent inhibition of TNFR- and/or FAS-mediated neuronal cell death, such as is observed in the early stages following spinal cord injury, is expected to promote nerve cell survival and regeneration.

Mitochondria membrane proteins (such as, Ndufv2, Atp5b, DLAT and Sdha) are involved in energy metabolism and are thought to be involved in cell-death signaling. Without being bound to theory, the binding of p45 to a variety of mitochondria membrane proteins is expected to modulate mitochondria energy metabolism (e.g., stimulate energy production) and inhibit mitochondrial death signaling in neuronal cells following spinal cord injury and, thereby, promote nerve cell survival and regeneration.

Cytoskeleton-regulating proteins (such as, N-Tmod and Rab-GDI) are involved in neurite outgrowth and axonal elongation. Without being bound to theory, p45 binding to cytoskeleton-regulating proteins is expected to enhance neurite outgrowth and axonal elongation; thereby, promoting p45-dependent nerve regeneration.

Munc18-1 (a.k.a. STXbp1 and p67) is involved in the vesicle transportation at nerve terminals. Without being bound to theory, p45 binding to Munc18-1 is expected to modulate release of neurotransmitters release, which can promote nerve cell survival after injury (such as, spinal cord injury).

The discovery of the foregoing p45 binding partners makes possible methods of screening for agents that affect complexes formed between p45 and one or more of such binding partners. Agents affecting such protein-protein interactions have the potential to also affect (such as promote) p45-dependent nerve regeneration. Accordingly, disclosed methods involve FADD, Ndufv2, Atp5b, DLAT, Sdha, N-Tmod, Rab-GDI, Munc18-1, and/or OFD1 polypeptides (such as naturally occurring proteins, p45-binding fragments thereof, and p45-binding variants thereof) and nucleic acid molecules encoding the same.

Representative FADD nucleic acid sequences and the corresponding encoded amino acid sequences are provided in GENBANK™ Accession Nos. NM_{—}010175 (Mus musculus nucleotide sequence), NP_{—}034305 (Mus musculus amino acid sequence), NM_{—}003824 (Homo sapiens nucleotide sequence), NP_{—}003815 (Homo sapiens amino acid sequence), NM_{—}152937 (Rattus norvegicus nucleotide sequence), NP_{—}690920 (Rattus norvegicus amino acid sequence), NM_{—}001031797 (Sus scrofa nucleotide sequence), NP_{—}001026967 (Sus scrofa amino acid sequence), NM_{—}001007816 (Bos taurus nucleotide sequence), and NP_{—}001007817 (Bos taurus amino acid sequence).

Representative Ndufv2 nucleic acid sequences and the corresponding encoded amino acid sequences are provided in GENBANK™ Accession Nos. NM_{—}021074 (Homo sapiens nucleotide sequence), NP_{—}066552 (Homo sapiens amino acid sequence), NM_{—}174565 (Bos taurus nucleotide sequence), NP_{—}776990 (Bos taurus amino acid sequence), XM_{—}981927 (Mus musculus variant 1 nucleotide sequence), XP_{—}987021 (Mus musculus variant 1 amino acid sequence), XM_{—}981966 (Mus musculus variant 2 nucleotide sequence), XP_{—}987060 (Mus musculus variant 2 amino acid sequence), XM_{—}001004994 (Mus musculus variant 3 nucleotide sequence), XP_{—}001004994 (Mus musculus variant 3 amino acid sequence), XM_{—}925211 (Mus musculus variant 4 nucleotide sequence), XP_{—}930304 (Mus musculus variant 4 amino acid sequence), NM_{—}031064 (Rattus norvegicus nucleotide sequence), and NP_{—}112326 (Rattus norvegicus amino acid sequence).

Representative Atp5b nucleic acid sequences and the corresponding encoded amino acid sequences are provided in GENBANK™ Accession Nos. NM_{—}016774 (Mus musculus nucleotide sequence), NP_{—}058054 (Mus musculus amino acid sequence), NM_{—}001686 (Homo sapiens nucleotide sequence), NP_{—}001677 (Homo sapiens amino acid sequence), NM_{—}134364 (Rattus norvegicus nucleotide sequence), NP_{—}599191 (Rattus norvegicus amino acid sequence), NM_{—}001031391 (Gallus gallus nucleotide sequence), NP_{—}001026562 (Gallus gallus amino acid sequence), NM_{—}175796 (Bos taurus nucleotide sequence), and NP_{—}786990 (Bos taurus amino acid sequence).

Representative DLAT nucleic acid sequences and the corresponding encoded amino acid sequences are provided in GENBANK™ Accession Nos. NM_{—}145614 (Mus musculus nucleotide sequence), NP_{—}663589 (Mus musculus amino acid sequence), NM_{—}001931 (Homo sapiens nucleotide sequence), NP_{—}001922 (Homo sapiens amino acid sequence), NM_{—}031025 (Rattus norvegicus nucleotide sequence), NP_{—}112287 (Rattus norvegicus amino acid sequence), NM_{—}213994 (Sus scrofa nucleotide sequence), NP_{—}999159 (Sus scrofa amino acid sequence), NM_{—}212667 (Danio rerio nucleotide sequence), and NP_{—}997832 (Danio rerio amino acid sequence).

Representative Sdha nucleic acid sequences and the corresponding encoded amino acid sequences are provided in GENBANK™ Accession Nos. NM_{—}023281 (Mus musculus nucleotide sequence), NP_{—}075770 (Mus musculus amino acid sequence), NM_{—}004168 (Homo sapiens nucleotide sequence), NP_{—}004159 (Homo sapiens amino acid sequence), NM_{—}130428 (Rattus norvegicus nucleotide sequence), NP_{—}569112 (Rattus norvegicus amino acid sequence), NM_{—}174178 (Bos taurus nucleotide sequence), and NP_{—}776603 (Bos taurus amino acid sequence).

Representative N-Tmod nucleic acid sequences and the corresponding encoded amino acid sequences are provided in GENBANK™ Accession Nos. NM_{—}016711 (Mus musculus nucleotide sequence), NP_{—}057920 (Mus musculus amino acid sequence), NM_{—}014548 (Homo sapiens nucleotide sequence), NP_{—}055363 (Homo sapiens amino acid sequence), XM_{—}535484 (Canis familiaris nucleotide sequence), XP_{—}535484 (Canis familiaris amino acid sequence), XP_{—}535484 (Pan troglodytes nucleotide sequence), XP_{—}523076 (Pan troglodytes amino acid sequence), XM_{—}610595 (Bos taurus nucleotide sequence), and XP-610595 (Bos taurus amino acid sequence).

Representative Rab-GDI nucleic acid sequences and the corresponding encoded amino acid sequences are provided in GENBANK™ Accession Nos. NM_{—}110273 (Mus musculus nucleotide sequence), NP_{—}034403 (Mus musculus amino acid sequence), NM_{—}001493 (Homo sapiens nucleotide sequence), Np_{—}001484 (Homo sapiens amino acid sequence), NM_{—}017088 (Rattus norvegicus nucleotide sequence), NP_{—}058784 (Rattus norvegicus amino acid sequence), NM_{—}001003185 (Canis familiaris nucleotide sequence), Np_{—}001003185 (Canis familiaris amino acid sequence), NM_{—}001009061 (Pan troglodytes nucleotide sequence), NP_{—}001009061 (Pan troglodytes amino acid sequence), NM_{—}174064 (Bos taurus nucleotide sequence), Np_{—}776489 (Bos taurus amino acid sequence), NM_{—}001020608 (Danio rerio nucleotide sequence), and NP_{—}001018444 (Danio rerio amino acid sequence).

Representative Munc18-1 nucleic acid sequences and the corresponding encoded amino acid sequences are provided in GENBANK™ Accession Nos. NM_{—}009295 (Mus musculus nucleotide sequence), NP_{—}033321 (Mus musculus amino acid sequence), NM_{—}001032221 (Homo sapiens nucleotide sequence), NP_{—}001027392 (Homo sapiens amino acid sequence), NM_{—}013038 (Rattus norvegicus nucleotide sequence), NP_{—}037170 (Rattus norvegicus amino acid sequence), NM_{—}206976 (Gallus gallus nucleotide sequence), NP_{—}996859 (Gallus gallus amino acid sequence), XM_{—}520272 (Pan troglodytes nucleotide sequence), XP_{—}520272 (Pan troglodytes amino acid sequence), NM_{—}174619 (Bos taurus nucleotide sequence), NP_{—}777044 (Bos taurus amino acid sequence), XM_{—}679277 (Danio rerio nucleotide sequence), and XP_{—}684369 (Danio rerio amino acid sequence).

Representative OFD1 nucleic acid sequences and the corresponding encoded amino acid sequences are provided in GENBANK™ Accession Nos. NM_{—}177429 (Mus musculus nucleotide sequence), NP_{—}803178 (Mus musculus amino acid sequence), NM_{—}003611 (Homo sapiens nucleotide sequence), NP_{—}003602 (Homo sapiens amino acid sequence), XM_{—}217615 (Rattus norvegicus nucleotide sequence), XP_{—}217615 (Rattus norvegicus amino acid sequence), XM_{—}529273 (Pan troglodytes nucleotide sequence), XP_{—}529273 (Pan troglodytes amino acid sequence), NM_{—}001004496 (Danio rerio nucleotide sequence), and NP_{—}001004496 (Danio rerio amino acid sequence).

FADD, Ndufv2, Atp5b, DLAT, Sdha, N-Tmod, Rab-GDI, Munc18-1, or OFD1 nucleic acid sequences, proteins, fragments and variants can be isolated and produced in the same manner as discussed in detail above for p45.

E. Heterologous Expression of Recombinant Polypeptides

Various commonly known expression systems, including eukaryotic and prokaryotic expression systems, or cell-free translation systems, are available for heterologous expression of p45 and p75 polypeptides useful in the disclosed compositions and methods.

Methods of expressing proteins in heterologous expression systems are well known in the art. Typically, a nucleic acid molecule encoding all or part of a protein of interest is obtained using methods such as those described herein. The protein-encoding nucleic acid sequence is cloned into an expression vector that is suitable for the particular host cell of interest using standard recombinant DNA procedures. Expression vectors include (among other elements) regulatory sequences (e.g., promoters) that can be operably linked to the desired protein-encoding nucleic acid molecule to cause the expression of such nucleic acid molecule in the host cell. Together, the regulatory sequences and the protein-encoding nucleic acid sequence are an “expression cassette.” Expression vectors may also include an origin of replication, marker genes that provide phenotypic selection in transformed cells, one or more other promoters, and a polylinker region containing several restriction sites for insertion of heterologous nucleic acid sequences.

Expression vectors useful for expression of heterologous protein(s) in a multitude of host cells are well known in the art, and some specific examples are provided herein. The host cell is transfected with (or infected with a virus containing) the expression vector using any method suitable for the particular host cell. Such transfection methods are also well known in the art and non-limiting exemplar methods are described herein. The transfected (also called, transformed) host cell is capable of expressing the protein encoded by the corresponding nucleic acid sequence in the expression cassette. Transient or stable transfection of the host cell with one or more expression vectors is contemplated by the present disclosure.

Many different types of cells may be used to express heterologous proteins, such as bacteria, yeasts, fungi, insects, vertebrate cells (such as mammalian cells), and plant cells, including (as appropriate) primary cells and immortal cell lines. Numerous representatives of each cell type are commonly used and are available from a wide variety of commercial sources, including, for example, ATCC, Pharmacia, and Invitrogen.

Further details of some specific embodiments are discussed below.

1. Prokaryotes

Prokaryotes, such as bacteria, may be used as host cells. Prokaryotic expression systems are advantageous, at least, because of culture affordability, ease of genetic manipulation, and high yields of desired product(s). Suitable prokaryotic host cells include, without limitation, E. coli K12 strain 94 (ATCC No. 31,446), E. coli strain W3 110 (ATCC No. 27,325), E. coli X1776 (ATCC No. 31,537), E. coli B, and many other strains of E. coli, such as HB101, JM101, NM522, NM538, NM539, B1-21, B1-21 (DE3), B1-21 (DE3) pLysS, Origami B, OmpT-defective CD41, CD43 (DE3), and phosphatidylenthanolamine (PE)-deficient AD93. Similarly, other species and genera of prokaryotes including bacilli such as Bacillus subtilis, other enterobacteriaceae, such as Salmonella typhimurium or Serratia marcesans, and various Pseudomonas species may all be used as prokaryotic expression hosts. Particular examples contemplate the use of protease attenuated bacterial host strains such as membrane protease OmpT-defective E. coli (Quick and Wright, Proc. Natl. Acad. Sci. USA, 99:8597-8601, 2002).

Prokaryotic host cells or other host cells with rigid cell walls may be transformed using any method known in the art, including, for example, calcium phosphate precipitation, or electroporation. Representative prokaryote transformation techniques are described in Dower (Genetic Engineering, Principles and Methods, 12:275-296, Plenum Publishing Corp., 1990) and Hanahan et al. (Meth. Enzymol., 204:63, 1991).

Vectors typically used for transformation of E. coli include, without limitation, pBR322, pUC18, pUC19, pUC118, pUC119, Bluescript M13 and derivatives thereof. Numerous such plasmids are commercially available and are well known in the art. Representative promoters used in prokaryotic vectors include the β-lactamase (penicillinase) and lactose promoter systems (Chang et al., Nature, 375:615, 1978; Itakura et al., Science, 198:1056, 1977; Goeddel et al., Nature, 281:544, 1979), a tryptophan (trp) promoter system (Goeddel et al., Nucl. Acids Res., 8:4057, 1980), and the alkaline phosphatase system.

2. Yeast

Various yeast strains and yeast-derived vectors are used commonly for the expression of heterologous proteins. For instance, Pichia pastoris expression systems, obtained from Invitrogen (Carlsbad, Calif.), may be used to express a p45 and/or p75 polypeptide. Such systems include suitable Pichia pastoris strains, vectors, reagents, transformants, sequencing primers, and media. Available strains include KM71H (a prototrophic strain), SMD1168H (a prototrophic strain), and SMD1168 (a pep4 mutant strain) (Invitrogen).

Saccharomyces cerevisiae is another yeast that is commonly used in heterologous expression systems. The plasmid YRp7 (Stinchcomb et al., Nature, 282:39, 1979; Kingsman et al., Gene, 7:141, 1979; Tschemper et al., Gene, 10:157, 1980) is commonly used as an expression vector in Saccharomyces. This plasmid contains the trp1 gene that provides a selection marker for a mutant strain of yeast lacking the ability to grow in tryptophan, such as strains ATCC No. 44,076 and PEP4-1 (Jones, Genetics, 85:12, 1977). The presence of the trp1 lesion as a characteristic of the yeast host cell genome then provides an effective environment for detecting transformation by growth in the absence of tryptophan.

Yeast host cells can be transformed using the polyethylene glycol method, as described by Hinnen (Proc. Natl. Acad. Sci. USA, 75:1929, 1978). Additional yeast transformation protocols are set forth in Gietz et al. (Nucl. Acids Res., 20(17): 1425, 1992) and Reeves et al. (FEMS, 99(2-3): 193-197, 1992).

Suitable promoting sequences in yeast vectors include the promoters for 3-phosphoglycerate kinase (Hitzeman et al., J. Biol. Chem., 255:2073, 1980) or other glycolytic enzymes (Hess et al., J. Adv. Enzyme Reg., 7:149, 1968; Holland et al., Biochemistry, 17:4900, 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In the construction of suitable expression vectors, the termination sequences associated with these genes are also ligated into the expression vector 3′ of the sequence desired to be expressed to provide polyadenylation of the mRNA and termination. Other promoters that have the additional advantage of transcription controlled by growth conditions are the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing yeast-compatible promoter, origin of replication and termination sequences is suitable.

3. Baculovirus-Infected Insect Cells

Another representative eukaryotic expression system involves the recombinant baculovirus, Autographa californica nuclear polyhedrosis virus (AcNPV; Summers and Smith, A Manual of Methods for Baculovirus Vectors and Insect Cell Culture Procedures, 1986; Luckow et al., Biotechnol., 6:47-55, 1987). Infection of insect cells (such as cells of the species Spodoptera frugiperda) with recombinant baculoviruses results in the expression of p45 and/or p75 polypeptides in the insect cells.

A baculovirus expression vector is prepared as previously described using standard molecular biology techniques. The vector may comprise the polyhedron gene promoter region of a baculovirus, the baculovirus flanking sequences necessary for proper crossover during recombination (the flanking sequences comprise about 200-300 base pairs adjacent to the promoter sequence) and a bacterial origin of replication which permits the construct to replicate in bacteria. In particular examples, the vector is constructed so that (i) a p45- or p75-encoding nucleic acid sequence is operably linked to the polyhedron gene promoter (collectively, the “expression cassette”) and (ii) the expression cassette is flanked by the above-described baculovirus flanking sequences.

Insect host cells (such as Spodoptera frugiperda cells) are infected with a recombinant baculovirus and cultured under conditions allowing expression of the baculovirus-encoded p45 or p75 polypeptide (including functional fragments or functional variants of either). The expressed protein may, if desired, be extracted from the insect cells using methods known in the art or as described herein.

4. Mammalian Cells

Mammalian host cells may also be used for heterologous expression of a p45 polypeptide (such as a non-primate p45 polypeptide or functional fragment or functional variant thereof), a p75 polypeptide, or one or more other p45 binding partners (such as, FADD, Ndufv2, Atp5b, DLAT, Sdha, N-Tmod, Rab-GDI, Munc18-1, and/or OFD1). Examples of suitable mammalian cell lines include, without limitation, monkey kidney CVI line transformed by SV40 (COS-7, ATCC™ CRL 1651); human embryonic kidney line 293S (Graham et al., J. Gen. Virol., 36:59, 1977); baby hamster kidney cells (BHK, ATCC™ CCL 10); Chinese hamster ovary cells (Urlab and Chasin, Proc. Natl. Acad. Sci USA, 77:4216, 1980); mouse sertoli cells (TM4, Mather, Biol. Reprod., 23:243, 1980); monkey kidney cells (CVI-76, ATCC™ CCL 70); African green monkey kidney cells (VERO-76, ATCC™ CRL-1587); human cervical carcinoma cells (HELA, ATCC™ CCL 2); canine kidney cells (MDCK, ATCC™ CCL 34); buffalo rat liver cells (BRL 3A, ATCC™ CRL 1442); human lung cells (W138, ATCC™ CCL 75); human liver cells (Hep G2, HB 8065); mouse mammary tumor cells (MMT 060562, ATCC™ CCL 5 1); rat hepatoma cells (HTC, MI.54, Baumann et al., J. Cell Biol., 85:1, 1980); and TRI cells (Mather et al., Annals N.Y. Acad. Sci., 383:44, 1982). Expression vectors for these cells ordinarily include (if necessary) DNA sequences for an origin of replication, a promoter located 5′ of the nucleic acid sequence to be expressed, a ribosome binding site, an RNA splice site, a polyadenylation site, and/or a transcription terminator site.

Promoters used in mammalian expression vectors can be of viral origin. Such viral promoters may be derived from polyoma virus, adenovirus 2, and simian virus 40 (SV40). The SV40 virus contains two promoters that are termed the early and late promoters. These promoters are useful because they are both easily obtained from the virus as one nucleic acid fragment that also contains the viral origin of replication (Fiers et al., Nature, 273:113, 1978). Smaller or larger SV40 DNA fragments may also be used, provided they contain the approximately 250-bp sequence extending from the HindIII site toward the BglI site located in the viral origin of replication. Alternatively, promoters that are naturally associated with the foreign gene (homologous promoters) may be used provided that they are compatible with the host cell line selected for transformation.

An origin of replication may be obtained from an exogenous source, such as SV40 or other virus (e.g., polyoma virus, adenovirus, VSV, BPV) and inserted into the expression vector. Alternatively, the origin of replication may be provided by the host cell chromosomal replication mechanism.

5. Cell-Free Translation

Cell-free translation systems are known in the art (see, e.g., Kurland, Cell, 28:201-202, 1982; Pavlov and Ehrenberg, Arch. Biochem. Biophys., 328:9-16, 1996), and can be used to synthesize p45 or p75 polypeptides useful in the disclosed compositions or methods. The most frequently used cell-free translation systems consist of extracts from rabbit reticulocytes, wheat germ, or E. coli. All are prepared as crude extracts containing all the macromolecular components (70S or 80S ribosomes, tRNAs, aminoacyl-tRNA synthetases, initiation, elongation and termination factors, etc.) required for translation of exogenous RNA. Each extract is supplemented with amino acids, energy sources (ATP, GTP), energy regenerating systems (creatine phosphate and creatine phospholinase for eukaryotic systems, and phosphoenol pyruvate and pyruvate kinase for the E. coli lysate), and other co-factors (Mg²⁺, K⁺, etc.) that facilitate the function of the particular translation machinery. Either DNA or RNA can be used as the starting material for cell-free protein synthesis. However, DNA starting material is necessarily transcribed to RNA using a “coupled” or “linked” system. A “linked” system generally involves DNA transcription with a bacteriophage polymerase followed by translation in the rabbit reticulocyte lysate or wheat germ lysate. E. coli translation systems are said to be “coupled” because transcription and translation occur simultaneously in E. coli.

Commercially available cell-free translation products (also referred to as in vitro translation products) and instructions for use may be purchased from AMBION (e.g., PROTEINSCRIPT-PRO™ Kit, Retic Lysate IVT™ Kit), ROCHE DIAGNOSTICS (e.g., RTS 500 PROTEOMASTER™ E. coli HY Kit, RTS 9000 E. coli HY Kit), QIAGEN (e.g., EASYXPRESS™ Protein Synthesis Kit), PROMEGA (e.g., TNT™ T7 Quick Coupled Transcription/Translation System), and numerous other suppliers.

IV. Methods for Promoting Nerve Regeneration

This disclosure illustrates (among other things) that disclosed p45 polypeptides (such as non-primate, naturally occurring p45 proteins, including those having the amino acid sequence shown in SEQ ID NOs: 2, 4, 6, 8, and 10, and functional fragments and variants thereof) can cause nerve cells to grow and/or sprout neuronal projections (such as axons or neurites) in vivo or in vitro, and/or increase neuronal survival, myelination of axons, synapse formation, or synaptic transmission. Accordingly, disclosed herein are methods of inducing nerve growth (in vivo or in vitro) by contacting a cell capable of neurite outgrowth (such as a nerve cell or other cell capable of neuronal differentiation; e.g., multipotent, pluripotent or totipotent stem cells) with a sufficient amount (e.g., a growth-promoting amount) of a p45 polypeptide or a nucleic acid encoding the p 45 polypeptide. Also disclosed are methods of promoting nerve regeneration by administering to a subject a therapeutically effective amount of a p45 polypeptide or a nucleic acid encoding the p45 polypeptide. p45 polypeptides and nucleic acids encoding p45 polypeptides useful in the disclosed methods have been described in detail above.

In particular method embodiments, nerve growth occurs in the context of recovery from damage to a neuron, a nerve, an axon, or other nervous system structure that includes axonal projection(s) of one or more neurons (such as one or more nerve tracts or the spinal cord itself). In some cases, damage involves the transection (complete or partial) or crush of one or more neuronal projections (such as axons); for example, complete transection, partial transection, or crush of one or more axons, neural tracts (including descending tracts, such as the CST, RST, and/or rubrospinal tract, or ascending tracts, such as sensory tracts, spinocerebellar tract, propriospinal neuronal system, and autonomic pathways), nerves (such as sciatic nerve), or even the spinal cord. Even more particular examples involve regeneration of nervous system (e.g., CNS) structures that, under untreated circumstances, do not substantially regenerate following damage, including, e.g., axons of CNS neurons (such as motor neurons), CNS nerve tracts (such as the CST and/or RST), or the spinal cord. Some exemplary nerve cells useful in a disclosed method express a p75 polypeptide (such as a p75 polypeptide naturally occurring in the subject cell(s), or a p75 polypeptide as set forth in SEQ ID NO: 18, SEQ ID NO: 24, or SEQ ID NO: 26). A nerve cell for use in other method embodiments will express one or more of CSPG, ephrin-B3, FADD, Ndufv2, Atp5b, DLAT, Sdha, N-Tmod, Rab-GDI, Munc18-1, and/or OFD1 (such as, CSPG and/or ephrin-B3, or FADD, Ndufv2, Atp5b, DLAT, Sdha, N-Tmod, Rab-GDI, Munc18-1, and/or OFD1).

In those examples involving a subject, nerve damage may have incurred as a result of a spinal cord injury, including a complete or partial spinal cord transection, or a crush injury of the spinal cord. Such spinal cord injury may occur at any level of the spinal cord, e.g., in one or more cervical, thoracic, or lumbar spinal cord vertebral segment(s). Particular examples involve injury in the thoracic spinal cord, such as at vertebral segment T8 or below, or vertebral segment T9 or below, or at vertebral segment T9/T10. Other examples involve injury in the lumbar spinal cord, such as at vertebral segment L1 or below, or vertebral segment L2 or below, or at vertebral segment L1/L2. In particular examples, a spinal cord injury results in quadriplegia or paraplegia in a subject. Any subject capable of sustaining nerve damage can be treated with a disclosed method. Exemplary subjects are mammals (such as mice, rats, bovine, canines, felines, ovines, equines, goats, any economically valuable farm mammal, or other commercially exploited mammal) and, in particular examples, humans.

Regeneration (or regrowth) in p45-treated cells (or subjects) can involve, for example, regeneration of one or more transected or crushed axons of one or more neurons (e.g., CNS neurons). Some examples involve regeneration of neural outgrowths in one or more CNS nerve tracts, including descending tracts, such as the CST, RST, and/or rubrospinal tract, or ascending tracts, such as sensory tracts, spinocrebellar tract, propriospinal neuronal system, and autonomic pathways). Particular examples involve regeneration of the CST and/or RST in a subject having incurred (e.g., prior to regeneration) a complete transection, partial transection, or crush injury of the spinal cord. Other examples involve regeneration of spinal motor neurons axons.

Nerve regeneration following contact with (or administration of) a p45 polypeptide or p45-encoding nucleic acid can be determined by any method known in the art. Neurite outgrowth and/or expression of axon-specific markers (such as neurotubulin, microtubule-associated protein 2, synaptophysin/synapsin/SV2, GAP-43, neurofilaments, or TuJ1) may be observed in some instances. Nerve regeneration also can be determined using a retrograde or anterograde tracer, for example a lipophilic tracer such as DiI, a biotin derivative (for example, biotin dextran amine), or a polar fluorescent dye, or a protein conjugate (for example, a lectin), a dextran conjugate, or fluorescent microspheres. Other means of monitoring nerve regeneration include electrophysiological recording, for example electrophysiological recording from a target muscle.

In some subjects, administration of p45 polypeptides or nucleic acids will cause a detectable improvement in the locomotor function of the subject as compared to an untreated subject (or the treated subject prior to treatment). For example, a subject can regain at least about 10%, at least about 25%, at least about 50%, or even at least about 90% of its locomotor function as compared to a control (e.g., an untreated subject or the same subject prior to p45 treatment).

An improvement in locomotor function can be determined by any available means, including, e.g., qualitative or quantitative assessments. Non-limiting examples of functional improvement can involve (among other things) (i) an ability (or an improvement in the ability) of an affected subject to support its weight on its limbs (e.g., hindlimbs or legs); (ii) an increase in muscle tone in limbs affected by the nerve damage; (iii) an ability (an improvement in the ability) of an affected subject to place one or more its limbs or extremities (e.g., hands and/or feet) in a position suitable for weight bearing and/or locomotion; (iv) an ability (or an improvement in the ability) of the subject to move from one place (e.g., stepping) to another or to move its limbs or extremities; (v) an ability (or an improvement in the ability) of the subject to maintain its balance; (vi) an ability (or an improvement in the ability) of the subject to coordinate intralimb joint movement(s); (vii) an ability (or an improvement in the ability) of the subject to maintain a substantially normal posture; (viii) an ability (or an improvement in the ability) of the subject to control gait pattern, organize the kinematics of each limb's swing, and/or control ground reaction forces applied during each stance phase; (ix) an ability (or an improvement in the ability) of the subject to control ankle movement; (x) an ability (or an improvement in the ability) of the subject to control plantar placing; (xi) an ability (or an improvement in the ability) of the subject to control stepping; (xii) an improvement in the subject's coordination; (xiii) an ability (or an improvement in the ability) of the subject to control paw position; (xiv) an ability (or an improvement in the ability) of the subject to control trunk instability; and/or (xv) an ability (or an improvement in the ability) of the subject to control tail position. Specific, non-limiting examples of measures of locomotor function in rodents include the rotarod test, the Basso, Beattie and Bresnahan (BBB) scale, and the Basso Mouse Scale (BMS).

V. Administration of Therapeutic Agents

This disclosure contemplates pharmaceutical compositions including one or more p45 polypeptides and/or one or more nucleic acids encoding such polypeptides, and further contemplates administering p45 therapeutics to subjects in need thereof, such as to subjects having a spinal cord injury. p45 polypeptides and corresponding nucleic acids useful in disclosed compositions and methods have been detailed above. Delivery systems and treatment regimens useful for such agents are known and can be used to administer these agents as therapeutics. In addition, representative embodiments are described below.

1. Administration of Nucleic Acid Molecules

In some embodiments where a therapeutic molecule is a nucleic acid encoding a therapeutic protein or peptide (for example, a nucleic acid molecule encoding a p45 polypeptide), or another type of therapeutic nucleic acid molecule (such as an siRNA, anti-sense oligonucleotide, ribozyme or other), administration of the nucleic acid may be achieved in a variety of ways. All forms of nucleic acid delivery are contemplated by this disclosure, including, without limitation, synthetic oligos, naked DNA, naked RNA (such as capped RNA), and plasmid or viral vectors (which may or may not be integrated into a target cell genome). For example, an expressible nucleic acid can be administered by use of a viral vector (see U.S. Pat. No. 4,980,286), or by direct injection, or by use of microparticle bombardment (for example, a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci., 88:1864-8, 1991). Alternatively, the expressible nucleic acid can be introduced into a host cell (such as a stem cell, e.g., a stem cell capable of neural differentiation) for expression of a polypeptide therapeutic in the host cell. In some examples, transfected/transformed host cells can be transplanted into a subject. In some instances, a nucleic acid can be incorporated within host cell DNA, for example, by homologous or non-homologous recombination, for stably expressing a therapeutic.

Expression vectors are commonly available that provide, for instance, constitutive, regulated, or cell/tissue-specific expression of a transcribable nucleic acid (e.g., a nucleic acid encoding a p45 polypeptide) included in the expression vector. All these vectors achieve the basic goal of delivering into the target cell a heterologous nucleic acid sequence and control elements needed for transcription. The vector pcDNA, which includes a strong viral promoter (CMV), is an example of an expression vector for constitutive expression of a heterologous DNA. Certain retroviral vectors (such as pRETRO-ON, Clontech) also use the constitutive CMV promoter but have the advantages of entering cells without any transfection aid, integrating into the genome of target cells only when the target cell is dividing. Regulated expression vectors include control elements that permit expression of an operably linked nucleic acid only when a corresponding regulator molecule (such as tetracycline or steroid hormones) is present. Exemplary regulated vectors include pMAM-neo (Clontech) or pMSG (Pharmacia), which use the steroid-regulated MMTV-LTR promoter, or pBPV (Pharmacia), which includes a metallothionein-responsive promoter. Numerous cell/tissue-specific expression vectors are also available for expression of heterologous nucleic acids in any of a variety of tissues or cell types. In some disclosed methods, it may be advantageous to use a nerve-specific expression vectors to express a p45 polypeptide primarily in neurons. Nerve-specific expression can be achieved using vectors including, for instance, any of the following nerve-specific promoters: Thy-1 promoter, peripheral myelin protein 22 gene promoter (Nelis et al., J. Med. Genet., 35(7):590-593, 1998), HB9 promoter, and Islet 1 promoter.

Viral vectors, which are derived from various viral genomes, are similarly numerous and commercially available. Exemplary viral vectors are derived from retroviruses (such as lentivirus), adenovirus, herpes simplex virus (HSV; Margolskee et al., Mol. Cell. Biol. 8:2837-2847, 1988), adeno-associated virus (McLaughlin et al., J. Virol. 62:1963-1973, 1988), polio virus and vaccinia virus (Moss et al., Annu. Rev. Immunol. 5:305-324, 1987). Representative retroviral vectors are derived from lentiviruses, Moloney murine leukemia virus (MoMuLV), Harvey murine sarcoma virus (HaMuSV), murine mammary tumor virus (MuMTV), and Rous Sarcoma Virus (RSV). Multiple teachings concerning viral vectors are available, e.g., Anderson, Science, 226:401-409, 1984; Hughes, Curr. Comm. Mol. Biol., 71:1-12, 1988; Friedman, Science, 244:1275-1281, 1989 and Anderson, Science, 256:608-613, 1992. Some viral vectors are replication-deficient and/or non-infective. Non-limiting representative neurotrophic viral vectors include herpes simplex viral vectors (see, e.g., U.S. Pat. No. 5,673,344) and adenoviral vectors (see, e.g., Barkats et al., Prog. Neurobiol., 55:333-341, 1998), or AAV or lentiviral vectors pseudotyped with rabies-G glycoptroein (Mazarakis et al., Human Mol. Genet., 10:2109-2121, 2001; Azzouz, et al., J. Neurosci., 22:10302-10312, 2002; Azzouz, et al., Nature, 429:413-417, 2004).

Other methods of delivery are also contemplated. For instance, lipidic and liposome-mediated gene delivery has recently been used successfully for transfection with various genes (for reviews, see Templeton and Lasic, Mol. Biotechnol., 11:175 180, 1999; Lee and Huang, Crit. Rev. Ther. Drug Carrier Syst., 14:173-206, 1997; and Cooper, Semin. Oncol., 23:172-187, 1996). For instance, cationic liposomes have been analyzed for their ability to transfect monocytic leukemia cells, and shown to be a viable alternative to using viral vectors (de Lima et al., Mol. Membr. Biol., 16:103-109, 1999). Such cationic liposomes can also be targeted to specific cells through the inclusion of, for instance, monoclonal antibodies or other appropriate targeting ligands (Kao et al., Cancer Gene Ther., 3:250-256, 1996).

2. Administration of Polypeptides or Peptides

In some embodiments, therapeutic agents comprising polypeptides or peptides may be delivered by administering to the subject a nucleic acid encoding the polypeptide or peptide, in which case the methods discussed in the section entitled “Administration of Nucleic Acid Molecules” should be consulted. In other embodiments, polypeptide or peptide therapeutic agents may be isolated from various sources and administered directly to the subject. For example, a polypeptide or peptide may be isolated from a naturally occurring source. Alternatively, a nucleic acid encoding the polypeptide or peptide may be expressed in vitro, such as in an E. coli expression system, as is well known in the art, and isolated in amounts useful for therapeutic compositions. Such methods are discussed in detail elsewhere in this specification.

A therapeutic polypeptide (such as a p45 polypeptide) for use in the pharmaceutical compositions can be modified according to procedures known in the art in order to enhance penetration of the blood-brain barrier. For example, U.S. Pat. No. 5,604,198 discloses that a molecule can be conjugated to a hydrophobic carrier which enhances the permeability of the blood brain barrier (BBB). WO 90/14838 teaches chemical modifications of a protein by increasing lipophilicity, altering glycosylation or increasing the net positive charge in order to enhance the BBB permeability of the protein.

3. Methods of Administration, Formulations and Dosage

Methods of administering a disclosed therapeutic include, but are not limited to, intrathecal, intradermal, intramuscular, intraperitoneal (ip), intravenous (iv), subcutaneous, intranasal, epidural, intradural, intracranial, intraventricular, and oral routes. A therapeutic may be administered by any convenient route, including, for example, infusion or bolus injection, topical, absorption through epithelial or mucocutaneous linings (for example, oral mucosa, rectal and intestinal mucosa, and the like) ophthalmic, nasal, and transdermal, and may be administered together with other biologically active agents. Administration can be systemic or local. In some instances, injection may be facilitated by a catheter, for example, attached to a reservoir. Pulmonary administration can also be employed (for example, by an inhaler or nebulizer), for instance using a formulation containing an aerosolizing agent.

In a specific embodiment, it may be desirable to administer a pharmaceutical composition locally to the area in need of treatment. This may be achieved by, for example, and not by way of limitation, local or regional infusion or perfusion during or following surgery, topical application (for example, wound dressing), injection, catheter, suppository, or implant (for example, implants formed from porous, non-porous, or gelatinous materials, including membranes, such as sialastic membranes or fibers), and the like. In one embodiment, a pump may be used (see, e.g., Langer Science 249, 1527, 1990; Sefton Crit. Rev. Biomed. Eng. 14, 201, 1987; Buchwald et al., Surgery 88, 507, 1980; Saudek et al., N. Engl. J. Med. 321, 574, 1989). In one specific example, administration is achieved by intravenous, intradural, intracranial, intrathecal, or epidural infusion of a therapeutic using a transplanted minipump. Such minipump may be transplanted in any location that permits effective delivery of the therapeutic agent to the target site; for instance, a minipump may be transplanted near a site of injury. In another embodiment, administration can be by direct injection at the site (or former site) of a tissue that is to be treated, such as a crushed or transected nerve. In another embodiment, a therapeutic is delivered in a vesicle, in particular liposomes (see, e.g., Langer, Science 249, 1527, 1990; Treat et al., in Liposomes in the Therapy of Infectious Disease and Cancer, Lopez-Berestein and Fidler (eds.), Liss, N.Y., pp. 353-365, 1989).

In yet another embodiment, a therapeutic agent can be delivered in a controlled release system. In another embodiment, polymeric materials can be used (see, e.g., Ranger et al., Macromol. Sci. Rev. Macromol. Chem. 23, 61, 1983; Levy et al., Science 228, 190, 1985; During et al., Ann. Neurol. 25, 351, 1989; Howard et al., J. Neurosurg. 71, 105, 1989). Other controlled release systems, such as those discussed in the review by Langer (Science 249, 1527 1990), can also be used.

The vehicle in which an agent is delivered can include pharmaceutically acceptable compositions known to those with skill in the art. For instance, in some embodiments, therapeutic agents disclosed herein are contained in a pharmaceutically acceptable carrier. The term “pharmaceutically acceptable” means approved by a regulatory agency of the federal or a state government or listed in the U.S. Pharmacopoeia or other generally recognized pharmacopoeia for use in animals, and, more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable, or synthetic origin, such as peanut oil, soybean oil, mineral oil, sesame oil, and the like. Water is a preferred carrier when the pharmaceutical composition is administered intravenously. Saline solutions, blood plasma medium, aqueous dextrose, and glycerol solutions can also be employed as liquid carriers, particularly for injectable solutions. The medium may also contain conventional pharmaceutical adjunct materials such as for example, pharmaceutically acceptable salts to adjust the osmotic pressure, lipid carriers such as cyclodextrins, proteins such as serum albumin, hydrophilic agents such as methyl cellulose, detergents, buffers, preservatives and the like.

Examples of pharmaceutical excipients include starch, glucose, lactose, sucrose, gelatin, malt, rice, flour, chalk, silica gel, sodium stearate, glycerol monostearate, talc, sodium chloride, dried skim milk glycerol, propylene, glycol, water, ethanol, and the like. The therapeutic, if desired, can also contain minor amounts of wetting or emulsifying agents, or pH buffering agents. The therapeutic can take the form of solutions, suspensions, emulsion, tablets, pills, capsules, powders, sustained-release formulations, and the like. The therapeutic can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and the like. A more complete explanation of parenteral pharmaceutical carriers can be found in Remington: The Science and Practice of Pharmacy (19th Edition, 1995) in chapter 95.

Embodiments of other pharmaceutical compositions are prepared with conventional pharmaceutically acceptable counterions, as would be known to those of skill in the art.

Therapeutic preparations will contain a therapeutically effective amount of at least one active ingredient, preferably in purified form, together with a suitable amount of carrier so as to provide proper administration to the patient. The formulation should suit the mode of administration.

Therapeutic agents of this disclosure can be formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection.

The ingredients in various embodiments are supplied either separately or mixed together in unit dosage form, for example, in solid, semi-solid and liquid dosage forms such as tablets, pills, powders, liquid solutions, or suspensions, or as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water or saline can be provided so that the ingredients may be mixed prior to administration.

The amount of the therapeutic that will be effective depends on the nature of the disorder or condition to be treated, as well as the stage of the disorder or condition. Effective amounts can be determined by standard clinical techniques. The precise dose to be employed in the formulation will also depend on the route of administration, and should be decided according to the judgment of the health care practitioner and each patient's circumstances. An example of such a dosage range is 0.001 to 200 mg/kg body weight in single or divided doses. Another example of a dosage range is 0.01 to 100 mg/kg body weight in single or divided doses. In some particular embodiments, a target concentration of a p45 polypeptide in a target cell or tissue is between 0.1 and 10 mg/kg body weight in single or divided doses.

The specific dose level and frequency of dosage for any particular subject may be varied and will depend upon a variety of factors, including the activity of the specific compound, the metabolic stability and length of action of that compound, the age, body weight, general health, sex, diet, mode and time of administration, rate of excretion, drug combination, and severity of the condition of the host undergoing therapy.

The therapeutic agents of the present disclosure can be administered at about the same dose throughout a treatment period, in an escalating dose regimen, or in a loading-dose regime (for example, in which the loading dose is about two to five times the maintenance dose). In some embodiments, the dose is varied during the course of a treatment based on the condition of the subject being treated, the severity of the disease or condition, the apparent response to the therapy, and/or other factors as judged by one of ordinary skill in the art. In some embodiments long-term treatment with a disclosed therapeutic is contemplated, for instance in order to have sustained expression or overexpression of a p45 polypeptide.

VI. Screening Methods

This disclosure has shown, among other things, that expression or overexpression of p45 and its variants (e.g., p45 functional fragments and various homologs) promotes nerve regrowth (such as regrowth of CNS nerves or axons). Further disclosed is that this p45 function is mediated, at least in part, via a previously unrecognized protein-protein interactions, for example, between p45 and p75 and/or between p45 and one or more of FADD, Ndufv2, Atp5b, DLAT, Sdha, N-Tmod, Rab-GDI, Munc18-1, and/or OFD1. These discoveries permit, for instance, methods for identifying agents (such as lead compounds or therapeutic agents) that affect p45 expression and/or function (e.g., p75-binding activity, FADD-binding activity, Ndufv2-binding activity, Atp5b-binding activity, DLAT-binding activity, Sdha-binding activity, N-Tmod-binding activity, Rab-GDI-binding activity, Munc18-1-binding activity, and/or OFD1-binding activity, or inhibition of nerve growth inhibitors (such as, CSPG, ephrin-B3, NogoA, MAG and/or CNS myelin). In some examples, such agents have the potential to promote nerve outgrowth (e.g., nerve regeneration), to be p45 mimetics (such as peptidomimetics), and/or to affect (e.g., increase or decrease) p45-p75 binding, p45-FADD binding, p45-Ndufv2 binding, p45-Atp5b binding, p45-DLAT binding, p45-Sdha binding, p45-N-Tmod binding, p45-Rab-GDI binding, p45-Munc18-1 binding, and/or p45-OFD1 binding.

A. Agents

Any agent that has potential (whether or not ultimately realized) to increase p45 expression (for instance in neurons), affect a p45 function (such as, enhance p45-dependent inhibition of nerve growth inhibitors, like CSPG, ephrin-B3, NogoA, MAG and/or CNS myelin), affect the interaction (in vivo or in vitro) between p45 and one or more of its specific-binding partners (such as, p75, FADD, Ndufv2, Atp5b, DLAT, Sdha, N-Tmod, Rab-GDI, Munc18-1, and/or OFD1) or otherwise be a p45 mimetic is contemplated for use in the methods of this disclosure. Such agents may include, but are not limited to, peptides such as for example, soluble peptides, including but not limited to members of random peptide libraries (see, e.g., Lam et al., Nature, 354:82-84, 1991; Houghten et al., Nature, 354:84-86, 1991), and combinatorial chemistry-derived molecular library made of D- and/or L-configuration amino acids, phosphopeptides (including, but not limited to, members of random or partially degenerate, directed phosphopeptide libraries; see, e.g., Songyang et al., Cell, 72:767-778, 1993), antibodies (including, but not limited to, polyclonal, monoclonal, humanized, anti-idiotypic, chimeric or single chain antibodies, and Fab, F(ab′)₂ and Fab expression library fragments, and epitope-binding fragments thereof), and small organic or inorganic molecules (such as so-called natural products or members of chemical combinatorial libraries).

Libraries (such as combinatorial chemical libraries) useful in the disclosed methods include, but are not limited to, peptide libraries (see, e.g., U.S. Pat. No. 5,010,175; Furka, Int. J. Pept. Prot. Res., 37:487-493, 1991; Houghton et al., Nature, 354:84-88, 1991; PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Natl. Acad. Sci. USA, 90:6909-6913, 1993), vinylogous polypeptides (Hagihara et al., J. Am. Chem. Soc., 114:6568, 1992), nonpeptidal peptidomimetics with glucose scaffolding (Hirschmann et al., J. Am. Chem. Soc., 114:9217-9218, 1992), analogous organic syntheses of small compound libraries (Chen et al., J. Am. Chem. Soc., 116:2661, 1994), oligocarbamates (Cho et al., Science, 261:1303, 1003), and/or peptidyl phosphonates (Campbell et al., J. Org. Chem., 59:658, 1994), nucleic acid libraries (see Sambrook et al. Molecular Cloning, A Laboratory Manual, Cold Springs Harbor Press, N.Y., 1989; Ausubel et al., Current Protocols in Molecular Biology, Green Publishing Associates and Wiley Interscience, N.Y., 1989), peptide nucleic acid libraries (see, e.g., U.S. Pat. No. 5,539,083), antibody libraries (see, e.g., Vaughn et al., Nat. Biotechnol., 14:309-314, 1996; PCT App. No. PCT/US96/10287), carbohydrate libraries (see, e.g., Liang et al., Science, 274:1520-1522, 1996; U.S. Pat. No. 5,593,853), small organic molecule libraries (see, e.g., benzodiazepines, Baum, C&EN, January 18, page 33, 1993; isoprenoids, U.S. Pat. No. 5,569,588; thiazolidionones and methathiazones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514) and the like.

Libraries useful for the disclosed screening methods can be produced in a variety of manners including, but not limited to, spatially arrayed multipin peptide synthesis (Geysen, et al., Proc. Natl. Acad. Sci., 81(13):3998-4002, 1984), “tea bag” peptide synthesis (Houghten, Proc. Natl. Acad. Sci., 82(15):5131-5135, 1985), phage display (Scott and Smith, Science, 249:386-390, 1990), spot or disc synthesis (Dittrich et al., Bioorg. Med. Chem. Lett., 8(17):2351-2356, 1998), or split and mix solid phase synthesis on beads (Furka et al., Int. J. Pept. Protein Res., 37(6):487-493, 1991; Lam et al., Chem. Rev., 97(2):411-448, 1997). Libraries may include a varying number of compositions (members), such as up to about 100 members, such as up to about 1000 members, such as up to about 5000 members, such as up to about 10,000 members, such as up to about 100,000 members, such as up to about 500,000 members, or even more than 500,000 members.

In one convenient embodiment, high throughput screening methods involve providing a combinatorial chemical or peptide library containing a large number of potential therapeutic compounds (e.g., potential nerve-regeneration agents, p45 mimetics, or affectors of p45-p75 interaction). Such combinatorial libraries are then screened in one or more assays as described herein to identify those library members (particularly chemical species or subclasses) that display a desired characteristic activity (such as increasing p45 expression, affecting p45-p75 interaction, or specific binding to a p45-specific antibody). The compounds thus identified can serve as conventional “lead compounds” or can themselves be used as potential or actual therapeutics. In some instances, pools of candidate agents may be identify and further screened to determine which individual or subpools of agents in the collective have the desired activity.

C. Assays

Screening methods may include, but are not limited to, methods employing solid phase, liquid phase, cell-based or virtual (in silico) screening assays. In some exemplary assays, compounds that affect the expression or a function of p45 (such as increase its expression or enhance its inhibition of nerve growth inhibitors) are identified. For instance, certain assays may identify compounds that bind to p45 gene regulatory sequences (e.g., promoter sequences) and which may modulate (e.g., increase) p45 gene expression (see, e.g., Platt, J. Biol. Chem., 269:28558-28562, 1994). Other representative assays identify compounds that interfere with or otherwise affect a protein-protein interaction between p45 and one or more of its binding partners (e.g., p75, FADD, Ndufv2, Atp5b, DLAT, Sdha, N-Tmod, Rab-GDI, Munc18-1, and/or OFD1), or compounds that are specifically recognized by an anti-p45 antibody (such as an antibody specific for a p75 binding domain of p45). Compounds identified via assays such as those described herein may be useful, for example, as neurogenic (or neurotrophic) drugs or to design and/or further identify neurogenic (or neurotrophic) drugs.

1. Agents that Modulate the Expression of a p45 Gene, Transcript or Polypeptide

Also disclosed herein are methods of identifying agents that modulate the expression of a p45 polypeptide or a nucleic acid encoding it (such as a p45 gene or transcript). Generally, such methods involve contacting (directly or indirectly) with a test agent an expression system comprising a nucleic acid sequence encoding a p45 polypeptide, or a reporter gene operably linked to a p45 transcription regulatory sequence, and detecting a change (e.g., an increase) in the expression of the p45-encoding nucleic acid or reporter gene. “Test agent” as used herein include all agents (and libraries of agents) described above.

Modulation of the expression of a p45 gene or gene product (e.g., transcript or protein) can be determined using any expression system capable of expressing a p45 polypeptide or transcript (such as a cell, tissue, or organism, or in vitro transcription or translation systems). In some embodiments, cell-based assays are performed. Non-limiting exemplary cell-based assays may involve test cells such as cells (including cell lines) that normally express a p45 gene, its corresponding transcript(s) and/or p45 protein(s), or cells (including cell lines) that have been transiently transfected or stably transformed with a reporter construct driven by a regulatory sequence of a p45 gene.

Exemplary cells (or tissues from which cells can be obtained) that normally express p45 transcripts and/or proteins include neurons, glial cells (such as oligodendrocytes, astrocytes, or microglial cells), cerebellar granule neurons, and embryonic stem cells.

As mentioned above, some disclosed methods involve cells (including cell lines) that have been transiently transfected or stably transformed with a reporter construct driven by a regulatory sequence of a p45 gene. A “regulatory sequence” as used herein can include some or all of the regulatory elements that regulate the expression of a particular nucleic acid sequence (such as a p45 gene) under normal circumstances. In particular examples, a regulatory region includes the contiguous nucleotides located at least 100, at least 500, at least 1000, at least 2500, at least 5000, or at least 7500 nucleotides upstream of the transcriptional start site of the regulated nucleic acid sequence (such as a p45 gene).

p45 gene regulatory regions are provided for a variety of species (including non-primate species, or non-primate, mammalian species) in publicly available genomic sequences. For example, mouse (Mus musculus) p45 is located on mouse chromosome 9 (location 9F2). In the mouse chromosome 9 contig (e.g., GENBANK™ Accession No. NT_{—}095756 (GI:63595357), version 2, last updated May 12, 2005 at 6:30 PM), the p45 initiator methionine (ATG) is located at (or at about) residue 1160467. Accordingly, a nucleic acid sequence of an upstream (i.e., 5′) regulatory region of the mouse p45 gene includes at least 100, at least 500, at least 1000, at least 2500, at least 5000, at least 7500, or at least 10,000 nucleotides upstream of residue 1160467 in GENBANK™ Accession No. NT_{—}095756 (version 2, last updated May 12, 2005 6:30 PM). In another example, human p45 (aka, LOC391534) is located on human chromosome 3 (location 3p21.31). In the Homo sapiens chromosome 3 genomic contig (e.g., GENBANK™ Accession No. NT_{—}022517 (GI:51464027), version 17, last updated Aug. 19, 2004 at 8:27 PM), the p45 initiator methionine (ATG) is located at (or at about) residue 46991244. Accordingly, a nucleic acid sequence of an upstream (i.e., 5′) regulatory region of the human p45 gene includes at least 100, at least 500, at least 1000, at least 2500, at least 5000, at least 7500, or at least 10,000 nucleotides upstream of residue 46991244 in GENBANK™ Accession No. NT_{—}022517 (GI:51464027, version 17, last updated Aug. 19, 2004 at 8:27 PM). In yet another example, rat (Rattus norvegicus) p45 is located on rat chromosome 8 (location 8q32). In the rat chromosome 8 genomic contig (e.g., GENBANK™ Accession No. NW_{—}047802 (GI:62654446), version 2, last updated Apr. 15, 2005 at 7:18 PM), the p45 initiator methionine (ATG) is located at (or at about) residue 1447737. Accordingly, a nucleic acid sequence of an upstream (i.e., 5′) regulatory region of the human p45 gene includes at least 100, at least 500, at least 1000, at least 2500, at least 5000, at least 7500, or at least 10,000 nucleotides upstream of residue 1447737 in GENBANK™ Accession No. NW_{—}047802 (GI:62654446, version 2, last updated Apr. 15, 2005 at 7:18 PM).

In method embodiments involving a cell transiently or stably transfected with a reporter construct operably linked to a p45 gene regulatory region, the level of the reporter gene product can be measured. Reporter genes are nucleic acid sequences that encode readily assayed proteins. Numerous reporter genes are commonly known and methods of their use are standard in the art. Non-limiting representative reporter genes are luciferase, β-galactosidase, chloramphenicol acetyl transferase, alkaline phosphatase, green fluorescent protein, and others. In the applicable methods, the reporter gene product is detected using standard techniques for that particular reporter gene product (see, for example, manufacturer's directions for human placental alkaline phosphatase (SEAP), luciferase, or enhance green fluorescent protein (EGPF) available from BDBiosciences (Clontech); or galactosidase/luciferase, luciferase, or galactosidase available from Applied Biosystems (Foster City, Calif., USA); or available from various other commercial manufacturers of reporter gene products). A difference in the level and/or activity of reporter gene measure in cells in the presence or absence of a test agent indicates that the test agent modulates the activity of the p45 regulatory region driving the reporter gene.

A change in the expression of a p45 gene (or a reporter gene), transcript or protein can be determined by any method known in the art. For example, the levels of a p45 (or reporter gene) transcript or protein can be measured by standard techniques, such as for RNA, Northern blot, PCR (including RT-PCR or q-PCR), in situ hybridization, or nucleic acid microarray, or, for protein, Western blot, antibody array, or immunohistochemistry. Alternatively, test cells can be examined to determine whether one or more cellular phenotypes have been altered in a manner consistent with modulation of expression of p45.

2. Agents that Affect the Interaction Between p45 and its Binding Partner(s)

As disclosed herein, p45 forms a protein-protein complex with one or more of p75, FADD, Ndufv2, Atp5b, DLAT, Sdha, N-Tmod, Rab-GDI, Munc18-1, and/or OFD1.

This disclosure demonstrates that a protein-protein interaction between p45 and p75 polypeptides inhibits p75 function in the NgR-mediated pathway that inhibits nerve regeneration. Agents that affect the p45-p75 protein-protein interaction (e.g, enhance formation of a p45-p75 complex, and/or increase the binding affinity of p45 and p75) may also have the effect of suppressing inhibition of nerve regrowth and, therefore, are desirable to identify. Similarly, p45 binds proteins involved in mitochrodrial mitochondrial signaling (e.g., energy metabolism and/or death signaling), cytoskeleton rearrangement of the neurite outgrowth, and synaptic vesicle transportation and synaptic transmission, including FADD, Ndufv2, Atp5b, DLAT, Sdha, N-Tmod, Rab-GDI, and Munc18-1. Such interactions are expected to contribute to (or mediate at least in part) p45-dependent nerve regeneration and p45-dependent functional recovery following spinal cord injury.

Agents that affect an interaction between p45 and one or more of its specific-binding partners (such as, p75) can be identified by a variety of assays, including solid-phase or solution-based assays. In a solid-phase assay, a p45 polypeptide (as described in detail elsewhere in this specification and, which includes, p75-binding fragments of p45 or p45 fragments that bind to FADD, Ndufv2, Atp5b, DLAT, Sdha, N-Tmod, Rab-GDI, Munc18-1, and/or OFD1) and one or more p45 binding partners (such as, a p75 polypeptide, as described in detail elsewhere in this specification and, which includes, p45-binding fragments of p75) are mixed under conditions in which p45 and its specific-binding partner (e.g., p75 FADD, Ndufv2, Atp5b, DLAT, Sdha, N-Tmod, Rab-GDI, Munc18-1, and/or OFD1) normally interact. For purposes of this paragraph, a p45 polypeptide and one or more of its specific-binding partners are each referred to as “a binding partner” and collectively are referred to as “binding partners.” One of the binding partners (e.g., a p45 polypeptide or its specific binding partner(s)) is labeled with a marker such as biotin, fluoroscein, EGFP, or enzymes to allow easy detection of the labeled component. The unlabeled binding partner is adsorbed to a support, such as a microtiter well or beads. Then, the labeled binding partner is added to the environment where the unlabeled binding partner is immobilized under conditions suitable for interaction between the two binding partners. One or more test compounds, such as compounds in one or more of the above-described libraries, are separately added to individual microenvironments containing the interacting binding partners. Agents capable of affecting the interaction between the binding partners are identified, for instance, as those that enhance retention or binding of the signal (i.e., labeled binding partner) in the reaction microenvironment, for example, in a microtiter well or on a bead for example. As discussed previously, combinations of agents can be evaluated in an initial screen to identify pools of agents to be tested individually, and this process is easily automated with currently available technology.

In still other methods, solution phase selection can be used to screen large complex libraries for agents that specifically affect protein-protein interactions (see, e.g., Boger et al., Bioorg. Med. Chem. Lett., 8(17):2339-2344, 1998); Berg et al., Proc. Natl. Acad. Sci., 99(6):3830-3835, 2002). In this example, each of two proteins that are capable of physical interaction (for example, p45 and p75 polypeptides or a p45 polypeptide and FADD, Ndufv2, Atp5b, DLAT, Sdha, N-Tmod, Rab-GDI, Munc18-1, and/or OFD1) are labeled with fluorescent dye molecule tags with different emission spectra and overlapping adsorption spectra. When these protein components are separate, the emission spectrum for each component is distinct and can be measured. When the protein components interact, fluorescence resonance energy transfer (FRET) occurs resulting in the transfer of energy from a donor dye molecule to an acceptor dye molecule without emission of a photon. The acceptor dye molecule alone emits photons (light) of a characteristic wavelength. Therefore, FRET allows one to determine the kinetics of two interacting molecules based on the emission spectra of the sample. Using this system, two labeled protein components are added under conditions where their interaction resulting in FRET emission spectra. Then, one or more test compounds, such as compounds in one or more of the above-described libraries, are added to the environment of the two labeled protein component mixture and emission spectra are measured. An increase in the FRET emission, with a concurrent decrease in the emission spectra of the separated components indicates that an agent (or pool of candidate agents) has affected (e.g., enhanced) the interaction between the protein components.

Interactions between p45 and one or more of its specific-binding partners (such as p75, FADD, Ndufv2, Atp5b, DLAT, Sdha, N-Tmod, Rab-GDI, Munc18-1, and/or OFD1) also can be determined (e.g., quantitatively or qualitatively) by co-immunoprecipitation of the relevant component polypeptides (e.g., from cellular extracts), by GST-pull down assay (e.g., using purified GST-tagged bacterial proteins), and/or by yeast two-hybrid assay, each of which methods is standard in the art. Conducting any one or more such assays in the presence and, optionally, absence of a test compound can be used to identify agents that affect (e.g., improve or enhance) the p45:specific-binding partner interaction in the presence of the test compound as compared to in the absence of the test compound or as compared to some other standard or control.

In certain method embodiments, one or more p75-binding fragments of p45 and/or one or more p45-binding fragments of p75 are used. p45 and p75 fragments having the desired binding activities have been previously discussed. In particular methods, a p45 polypeptide includes at least 15 (such as at least 25, at least 50, at least 100, or at least 150) consecutive amino acids of SEQ ID NO: 2, 4, 6, or 8 (or a polypeptide having 90% sequence identity with any of such sequences). Preferably, these consecutive amino acids will also include the p-75 binding domain of p45. Similarly, in other method embodiments, a p75 polypeptide includes at least 15 (e.g., at least 25, at least 50, at least 100, or at least 150) consecutive amino acids of SEQ ID NO: 18, SEQ ID NO: 24, SEQ ID NO: 26, (or a polypeptide having 90% sequence identity with any of such sequences). Preferably, these consecutive amino acids will also include the p45 binding domain of p75 (e.g., residues 360-377 of SEQ ID NO: 18, residues 362-379 of SEQ ID NO: 24, or residues 362-379 of SEQ ID NO: 26).

The disclosed methods contemplate the use of a p45 or p75 polypeptide or one or more FADD, Ndufv2, Atp5b, DLAT, Sdha, N-Tmod, Rab-GDI, Munc18-1, and/or OFD1 polypeptides contained, independently, in a cell or cellular extract, or as an isolated polypeptide. Certain methods embodiments involve a further step of determining whether an agent that affects the p45-p75 interaction also specifically binds to a p75 polypeptide.

In particular methods, the formation of a p45:specific-binding partner (e.g., p75, FADD, Ndufv2, Atp5b, DLAT, Sdha, N-Tmod, Rab-GDI, Munc18-1, and/or OFD1) complex is enhanced or increased when the amount of such complex is at least 20%, at least 30%, at least 50%, at least 100% or at least 250% higher than a control measurement (e.g., in the same test system prior to addition of a test agent, or in a comparable test system in the absence of a test agent). In other methods, the formation of a p45:specific-binding partner (e.g., p75, FADD, Ndufv2, Atp5b, DLAT, Sdha, N-Tmod, Rab-GDI, Munc18-1, and/or OFD1) complex is inhibited when the amount of such complex is at least 20%, at least 30%, at least 50%, at least 100% or at least 250% less than a control measurement (e.g., in the same test system prior to addition of a test agent, or in a comparable test system in the absence of a test agent). In some methods, inhibition of a p45:specific-binding partner interaction may be nearly complete such that substantially no protein-protein complex involving p45 and that particular specific binding partner is detected using traditional detection methods.

C. Identifying Agents that Affects a p45 Function

p45 expression or overexpression results in regeneration of axotomized spinal nerve tracts and remarkable functional recovery in living subjects with spinal cord injury (e.g., complete spinal transection). In addition, this disclosure has demonstrated that p45 inhibits MAG-Fc-induced RhoA activation and CSPG- and ephrin-B3-dependent inhibition of neurite outgrowth. Accordingly, it is desirable to identify agents having the potential to enhance or improve one or more of these p45 functions (e.g., promote p45-dependent nerve outgrowth (and/or growth cone development)), at least, because such agents are candidates for neurogenic (or neurotrophic) therapeutics. Exemplary assays to identify such agents can involve detecting a p45-dependent functional (e.g., phenotypic) difference in an in vitro or in vivo assay system.

Representative assay systems that can be used to measure p45-dependent activity in the presence or absence of test agents include neurite outgrowth and growth cone assays. Neurite outgrowth assays and assays for the development of growth cones are commonly known (see, e.g., U.S. Pat. Nos. 6,821,993 and 6,838,477; Huang et al., Neuron, 24:639-647, 1999; McKerracher et al., Neuron, 13:805, 1994; Mukhopadhyay et al., Neuron, 13:757-767, 1994; Chen et al., Nature, 403:434-439, 2000; GrandPre et al., Nature, 403:439-444, 2000; Prinjha et al., Nature, 403:383-384, 2000). In these embodiments, the assay system is capable of undergoing the desired phenotypic change, e.g., neurite outgrowth or development of growth cones. Accordingly, certain cell-based systems are suitable for conducting such assays. For example, neurons (such as cerebellar granular neurons, spinal motor neurons, cortical neurons, or spinal sensory neurons) can be used. In particular embodiments, the same type of cell is used for test and control assay systems. In other embodiments, cells of test and control assay systems express a p75 polypeptide and, in specific embodiments, substantially the same amount of a p75 polypeptide.

To ensure that an observed phenotype is attributable to a p45 polypeptide, a control assay system will express substantially no p45 (e.g., undetectable by Western blot) or substantially less p45 as compared to a non-control assay system. In this context, substantially less means at least 25% less, at least 50% less, at least 75%, or at least 90% less p45 in the control versus non-control assay system. A non-control assay system expresses or overexpresses p45 (or otherwise is treated to have more p45) as compared to control (e.g., at least 10%, at least 25%, at least 50%, at least 75%, or at least 90% more p45 expression than control). In some examples, such expression or overexpression is achieved by transfecting one or more cells with an expression vector encoding the p45 polypeptide. In other examples, a p45-deficient or substantially p45-cell is a neuron (such as a primary neuron) from a control (e.g., non-p45 transgenic) mouse, and a p45-expressing or p45-overexpressing cell is a neuron from a p45-transgenic mouse (see, e.g., Example 5). In some examples, a GST-p45 fusion protein can be expressed either in a transfected cell or transgenic animal. The GST module of such fusion protein permits rapid identification of p45-expressing cells.

One or more test agents are contacted to the control and non-control assay systems (e.g., cells of such assay systems), and a p45-dependent phenotype (such as neurite outgrowth or growth cone development) is detected. An agent having potential to promote nerve outgrowth is one for which neurite outgrowth or growth cone development is greater in the non-control, p45 expressing or overexpressing system. For instance, in one specific non-limiting example, GFP-positive p45-overexpressing neurons isolated from transgenic mice (e.g., expressing a heterologous GFP-p45 fusion protein) are cultured on myelin-inhibitor-coated plates in the presence of test compounds. Compounds are identified that enhance or attenuate p45-dependent improvement in neurite outgrowth when compared to control neurons. The GFP marker permits this assay to be used in a high-throughput automatic screening format using an imaging system.

In some cell-based method embodiments described here and throughout the specification, test cells or test agents can be presented in a manner suitable for high-throughput screening; for example, one or a plurality of test cells can be seeded into wells of a microtitre plate, and one or a plurality of test agents can be added to the wells of the microtitre plate. Alternatively, one or a plurality of test agents can be presented in a high-throughput format, such as in wells of microtitre plate (either in solution or adhered to the surface of the plate), and contacted with one or a plurality of test cells under conditions that, at least, sustain the test cells. Test agents can be added to test cells at any concentration that is not lethal to the cells. It is expected that different test agents will have different effective concentrations. Thus, in some methods, it is advantageous to test a range of test agent concentrations.

In particular methods, a function of a p45 polypeptide (e.g., promotion of nerve outgrowth (and/or growth cone development, inhibition of MAG-Fc-induced RhoA activation and/or inhibition of CSPG- and ephrin-B3-dependent inhibition of neurite outgrowth) is enhanced, improved, or increased when a quantitative or qualitative measure of such function is at least 20%, at least 30%, at least 50%, at least 100% or at least 250% higher than a control measurement (e.g., in the same test system prior to addition of a test agent, or in a comparable test system in the absence of a test agent).

4. p45 Mimotopes

As known in the art, antibodies can recognize epitopes having a particular structure (as opposed to having a particular primary sequence). Such epitopes are referred to as conformational epitopes. Accordingly, p45-specific antibody can be used to identify agents that mimic the structure of an original p45 epitope (such agents are referred to in the art as “mimotopes” (see, e.g., Rudolf et al., J. Immunol., 160:3315-3321, 1998) or herein may also be referred to as “mimetics” or “peptidomimetics”). An agent having a structure that mimics a p45 polypeptide (such as a p75-binding fragment of p45) potentially has a desired activity of the original p45 polypeptide (such as a nerve-regenerating and/or p75-binding activity(ies)).

The basic principle of assays used to identify agents bound by an anti-p45 antibody involves contacting one or more agents (such as a library of agents) with an anti-p45 antibody (such as an antibody specific for peptide including the p75-binding domain) under conditions and for a time sufficient to allow the two components to interact and bind, thus forming a complex which can be detected. In some instances, an agent that selectively binds to an anti-p45 antibody is selected for further testing for its ability to promote nerve regeneration and/or bind to p75.

The antibody binding assays can be conducted in a variety of ways, which are very well known in the art. For example, one method to conduct such an assay would involve anchoring one or more anti-p45 antibodies (including antigen-binding fragments of an anti-p45 antibody) or test substances onto a solid phase and detecting antibody-test compound complexes anchored on the solid phase at the end of the reaction. In one embodiment of such a method, a library of test agents may be anchored onto a solid surface (such as a microarray or in a microtiter plate), and an anti-p45 antibody, which is not anchored, can be labeled, either directly or indirectly, for detection when forming a complex with one or more members of the immobilized library.

Antibodies that recognize a p45 epitope (e.g., conformational epitopes) may be monoclonal or polyclonal; although, monoclonal antibodies are preferred. Monoclonal or polyclonal antibodies may be produced to specifically recognize and bind a p45 polypeptide (e.g., SEQ ID NO: 2, 4, 6, 8, or 10) or fragments thereof (such as p75-binding domains (e.g., residues 161-178 of SEQ ID NO: 2), TM/ICD, ICD, or others). Monoclonal or polyclonal antibody to a p45 polypeptide can be prepared, for example, using any of the detailed procedures described in Harlow and Lane (Antibodies, A Laboratory Manual, New York: Cold Spring Harbor Laboratory Press, 1988). In specific examples, a monoclonal antibody to an epitope of a p45 polypeptide can be prepared from murine hybridomas according to the classical method of Kohler and Milstein (Nature, 256:495-497, 1975) or derivative methods thereof.

The determination that an antibody specifically detects a p45 polypeptide (such as a p75-binding domain of p45) is made by any one of a number of standard immunoassay methods; for instance, the Western blotting technique (Sambrook et al., Molecular Cloning: A Laboratory Manual, New York: Cold Spring Harbor Laboratory Press, 1989).

### EXAMPLES

The following examples are provided to illustrate certain particular features and/or embodiments. These examples should not be construed to limit the invention to the particular features or embodiments described.

### Example 1

### Heterologously Expressed p45 and p75 Form a Stable Protein-Protein Complex

This Example illustrates that p45 forms a stable complex with p75 through the p45 TM and IC domains. The p45-p75 complex is believed to antagonize myelin inhibitor-mediated signaling by prevent the coupling of p75 with NgR. This discovery is in contrast to previously known mechanisms whereby receptor-mediated signaling is antagonized through the ECDs of TNFR decoy receptors, which prevent ligands from binding the functional receptors (see, e.g., Onichtchouk et al., Nature, 401:480, 1999; Pan et al., Science, 277:815, 1997; Sheridan et al., Science, 277:818, 1997).

Because p45 and p75 share similar TM and IC domains, including the DD, immunoprecipitation experiments were performed to determine whether p45 and p75 formed a stable protein-protein complex. Heterologous HEK293 cells were co-transfected with expression vectors for the full-length p45 and a c-Myc-tagged, full-length p75 fusion protein. Anti-c-myc antibodies specific for the c-Myc-p75 fusion were used to immunoprecipitate from transfected HEK293 lysates c-Myc-p75 together with any stably associated proteins. As shown in FIG. 1B, full-length p45 (p45-FL) and c-Myc-tagged p75 form a stable immunocomplex.

To further define the region of p75 involved in the p45-p75 protein-protein interface, HEK293 cells were co-transfected with expression vectors encoding a Flag-tagged, truncated p75, which lacked the p75 ECD (“Flag-p75-TM-ICD”), and full-length p45. An anti-Flag antibody immunoprecipitated Flag-p75-TM-ICD and full-length p45 (see FIG. 1C). Full-length and truncated forms of p75 were similarly efficient at forming a complex with full-length p45. Collectively, the foregoing results indicate that p45 forms a stable protein-protein complex with p75 primarily through the p75 TM and/or ICD.

Because Troy forms a complex with NgR and may substitute for p75 in NgR signaling (Park et al., Neuron, 45:345, 2005; Shao et al., Neuron, 45:353, 2005), immunoprecipitation experiments were performed to determined whether p45 also binds Troy. p45 and Troy did not form a stable protein-protein complex. It is notable that, in contrast to p75, Troy does not have a DD.

The ability of p45 TM and IC domains to antagonize the function of a related receptor (i.e., p75) in a ligand-independent manner is new. One other ligand-dependent system was previously described: Binding of Slit to Robo 1 leads to an ICD interaction between Robo 1 and DCC and, thereby, silences attraction signaling mediated by netrin through DCC (Stein and Tessier-Lavigne, Science, 291:1928, 2001). Recently, Robo 3/R1g-1, a new member of the Robo family and lacking some important cytoplasmic motifs found in other Robo family, was found to antagonize Robo 1-mediated repulsion signals, thereby preventing Robo 1 from interfering with netrin signaling through DCC and leading to attraction of axons (Sabatier et al., Cell, 117:157, 2004). However, the inhibition of Robo 1 by Robo 3 is dependent upon Slit binding.

### Example 2

### Analysis of p45/p75 ICD Complex Formation by TROSY-NMR

The interaction between p45 and p75 ICDs was further characterized using transverse relaxation-optimized spectroscopy (TROSY)-NMR. This Example demonstrates that ICDs of p45 and p75 interact directly, through mostly surface residues of α helix3 (α3) of p45ICD and part of α2 (as shown in FIG. 1D).

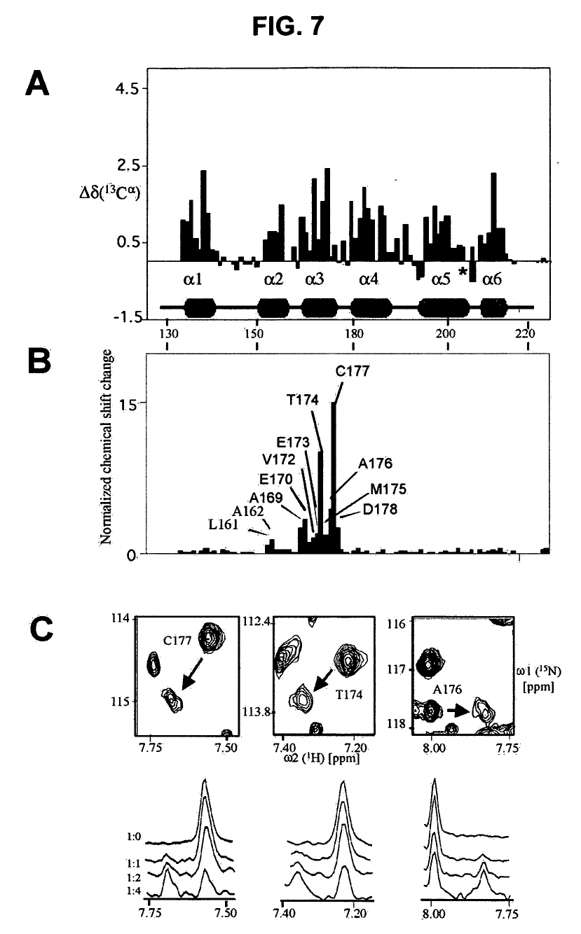

As a prerequisite for the identification of the binding interface, complete sequence specific NMR assignment of p45ICD was determined (FIG. 7A). The binding interface was mapped by measuring progressive changes in [¹H, ¹⁵N]-TROSY spectra of ¹³C, ¹⁵N-labeled p45ICD following stepwise addition of unlabeled p75ICD (FIGS. 7B and C). A change in the [¹H, ¹⁵]-TROSY indicates a perturbation of the chemical environment of residues of p45ICD due to a complex formation with p75ICD, hence the surface of p45ICD involved in contact with p75ICD can be determined. Superposition of the [¹H, ¹⁵N,]-TROSY spectra of p45ICD in the absence and in the presence of p75ICD revealed that a large number of peaks in the complex superimposed exactly with peaks in free p45ICD. This observation indicates that the overall fold of free p45ICD was conserved in the complex with p75ICD. However, relative intensities as well as the peak positions of some well-resolved peaks in free and complexed p45ICD were significantly different (FIGS. 7B and C), indicative of complex formation. The titration experiments enabled the transfer of sequence-specific resonance assignments of free [¹³C, ¹⁵N] p45ICD to [¹³C, ¹⁵N]-p45ICD in complex with unlabeled p75ICD. Thus, ¹⁵N-¹H-moieties of L161, A162, A169, E170, V172, E173, T174, M175, A176, C177 and D178 were found to exhibit chemical shift changes relative to free p45ICD of 0.0 to 1.0 ppm along the ¹⁵N dimension and 0.0 to 0.25 ppm along the ¹H dimension in the 1:4 complex (FIGS. 7B and C). Most of the observed changes corresponded to residues located at the helix3 of p45ICD (A169, E170, V172, E173, T174, M175, A176, C177), with some at helix2 (L161, A162) and the loop between helix3 and helix4 (D178) of the DD of p45. Residues T174, A176 and C177, exhibited large chemical shift changes, indicating that these residues participated strongly in the interaction. The observed chemical shift perturbation of E170, E173 and D178 indicates an involvement of electrostatic interactions.

### Example 3

### p45 Forms a Stable Protein-Protein Complex with p75 in Vivo and Interferes with p75/NgR-Mediated RhoA Activation

This Example demonstrates that p45 and p75 form a complex in vivo, and that p45 competes with NgR for binding to p75 and, thereby, interfere with inhibitory signaling through the p75/NgR complex.

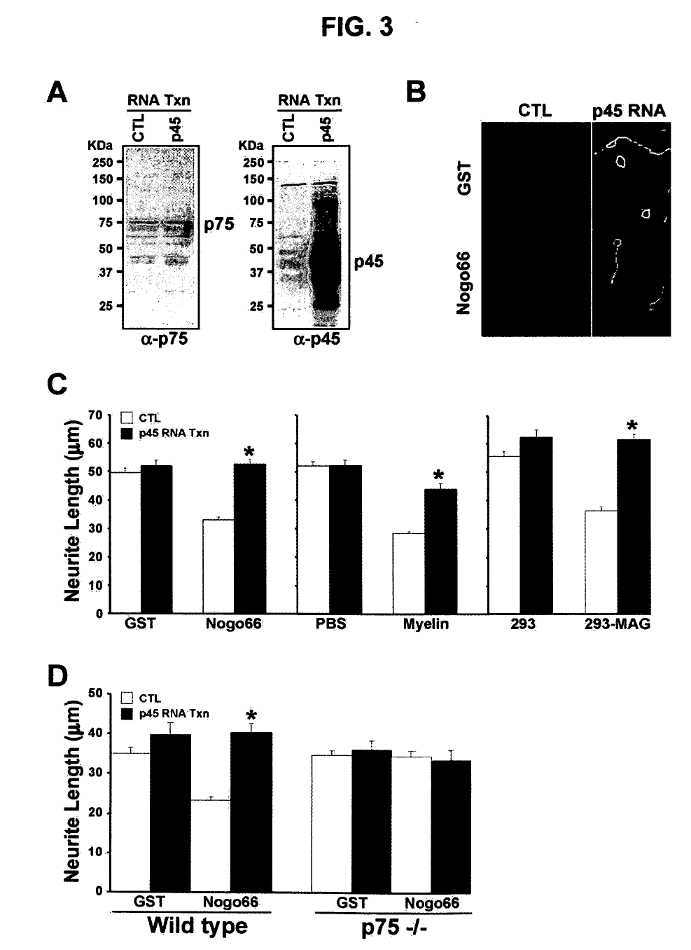

Both p45 and p75 are normally expressed in the cerebellum (see FIG. 3a). Cerebellar proteins from postnatal day 7 (P7) mice were immunoprecipitated with anti-p45 antibodies, followed by immunoblotting with anti-p75 antibodies. As shown in FIG. 1e, p45 and p75 form an immunocomplex in vivo in the cerebellum.

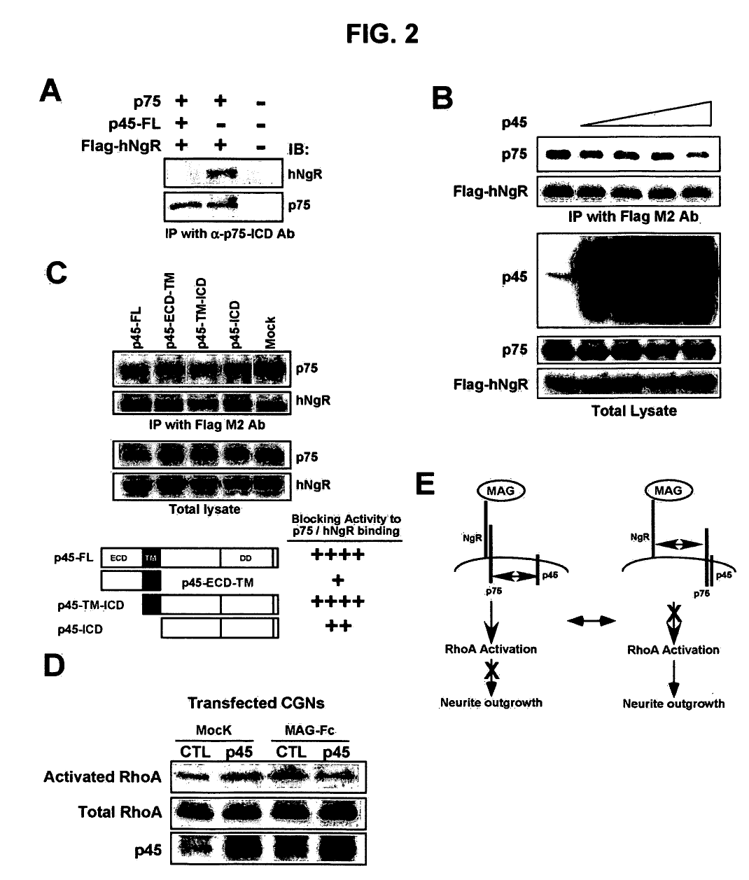

To demonstrate that the formation of p45-p75 complexes in vitro (see Examples 1 and 2) and in vivo interferes with p75/NgR complex formation, HEK293 cells were co-transfected with p75 and Flag-tagged human NgR (Flag-hNgR) expression constructs in the presence or absence of a p45 expression construct. Lysates from the transfected cells were immunoprecipitated with either anti-p75ICD or anti-Flag antibodies. As shown in FIG. 2A, p45 expression markedly reduced the level of complex formation between p75 and NgR. When increasing amounts of p45 expression vector were co-transfected with fixed amounts of p75 and Flag-hNgR expression vectors, a dose-dependent decrease in the formation of a p75-NgR complex was observed (see FIG. 2B).

The regions of p45 that are involved in the interference by p45 with the formation of p75/NgR complex were determined using p45 constructs containing deletions of ICD (“p45-ECD-TM”; corresponding to residues 1-222 of SEQ ID NO: 1 and residues 1-74 of SEQ ID NO: 2), ECD (“p45-TM-ICD”; corresponding to residues 157-687 of SEQ ID NO: 1 and residues 53-228 of SEQ ID NO: 2), or both ECD and TM (“p45-ICD”; corresponding to residues 223-687 of SEQ ID NO: 1 and residues 75-228 of SEQ ID NO: 2) (see schematic representations in FIG. 2C (bottom)). Expression vectors encoding each of p45-ECD-TM, p45-TM-ICD, or p45-ICD were co-transfected into HEK293 cells with p75 and NgR expression constructs. As shown in FIG. 2C, p45 without the ECD (p-TM-ICD) displayed equal ability as full-length p45 to interfere with the p75-NgR interaction. In contrast, p45-ICD and p45-ECD-TM were less efficient at interfering with p75-NgR complex formation (FIG. 2C). These results indicated that at least part of each of the TM and ICD of p45 are required for efficiently interfering with the complex formation between p75 and NgR.

Previous results have demonstrated that p75 is required for MAG-induced RhoA activation through NgR (Yamashita et al., J. Cell Biol., 157(4):565-570, 2002; Wang et al., Nature, 420:74, 2002; Wong et al., Nat. Neurosci., 5:1302, 2002; Dubreuil et al., J. Cell. Biol., 162:233, 2003). RhoA activation is necessary for neurite outgrowth inhibition mediated by myelin-derived inhibitors (Fournier et al., J. Neurosci., 23:1416, 2003). Thus, the effect of p45 over-expression on RhoA activation mediated by the p75/NgR complex was determined.

P7 cerebellar granule neurons (CGNs) that express low levels of endogenous p45 were transfected with full-length, capped p45 RNAs containing poly (A) tail generated by an in vitro transcription system. Advantageously, p45 RNA may be locally translated without an intervening transcription step which may necessitate transport of the encoding nucleic acid to the neuronal cell body. A transfection efficiency of about 50-70% was observed using the above-described p45 RNA.

As shown in FIG. 2D, the levels of p45 proteins were markedly elevated 24 hours following RNA transfection as compared to levels of endogenous (e.g., pre-transfection levels of) p45 proteins. When a Flag-tagged p45 construct was used to generate RNA for transfection, similar results were observed. The CGN cultures were then serum-starved and treated with Fc or MAG-Fc proteins. The levels of activated RhoA were measured. As illustrated in FIG. 2d, over-expression of p45 blocks MAG-Fc-induced RhoA activation. These results indicated that p45 can effectively block RhoA activation. FIG. 2E shows one possible, non-binding mechanism for p45-mediated RhoA inactivation, in which p45 interferes with formation of the p75-NgR complex and, thereby, inhibits RhoA activation. In the absence of inhibitory signals mediated by RhoA, neurite outgrowth can occur.

Although not bound by theory, the binding of p45 to p75 may interfere with the coupling of NgR with p75 by, for example, (i) causing a conformational change in the p75ECD that in turn reduces the ability of p75ECD to bind NgR; (ii) sequestering p75 so that the proportion of the p75/NgR complex is decreased in the presence of p45; or (iii) it has been shown that p75ECD can form a homodimer (He and Garcia, Science, 304:870, 2004), which might be a preferred partner (as compared to a p45/p75 complex) for interacting with NgR.

### Example 4

### p45 Promotes Nerve Outgrowth in the Presence of Myelin-Associated Inhibitors

This Example demonstrates that over-expression of p45 prevents neurite-outgrowth inhibition induced by NogoA, MAG or CNS myelin.

P7 CGNs were transfected with p45 RNA, plated onto coverslips coated with different substrates (i.e., GST, Nogo66, PBS, Myelin, HEK293 cells, or HEK293 cells expressing MAG, as indicated in FIGS. 3B and 3D), and allowed to grow overnight p45 RNA transfection markedly elevated p45 protein levels, yet the levels of endogenous p75 protein were not altered (see FIG. 3A). The cultures were double immunostained with antibodies against p45 and neurotubulin (TuJ1) (see FIG. 3B). Control and p45-transfected CGNs were cultured on control or inhibitory substrates and immunostained with rabbit antibodies against p45 and mouse monoclonal antibodies against neurotubulin (TuJ1) followed by Cy3-conjugated anti-rabbit and fluorescein-conjugated anti-mouse secondary antibodies, respectively.

The neurite lengths (TuJ1 positive) of control CGNs and p45-transfected CGNs, which displayed increased p45 immunoreactivity over control CGNs, were measured. As shown in FIG. 3C, neurite-outgrowth inhibition elicited by Nogo66 was alleviated by p45 over-expression. Similarly, p45 over-expression significantly promoted neurite outgrowth that was otherwise inhibited when cultured on coverslips coated with CNS myelin or MAG-expressing cells (see FIG. 3C).

To determine whether p45 specifically blocks p75-dependent inhibition, CGNs isolated from control and p75 mutant mice (Lee et al., Cell, 69:737, 1992) were transfected with p45 RNA and cultured on coverslips coated with GST or GST-Nogo66. Consistent with previous results (Yamashita et al., J. Cell Biol. 157:565, 2002; Wang et al., Nature, 420:74, 2002), Nogo66 failed to significantly inhibit neurite outgrowth in p75-deficient CGNs (FIG. 3d). Interestingly, over-expression of p45 did not enhance neurite outgrowth of p75-deficient CGNs. These results demonstrate that p45 promotes neurite outgrowth, at least in part, by specifically blocking p75-dependent signaling.

### Example 5

### p45 Promotes Rapid Functional Recovery Following Complete Spinal Cord Transection

This Example demonstrates that p45 expression promotes nerve regeneration following spinal cord injury (SCI).

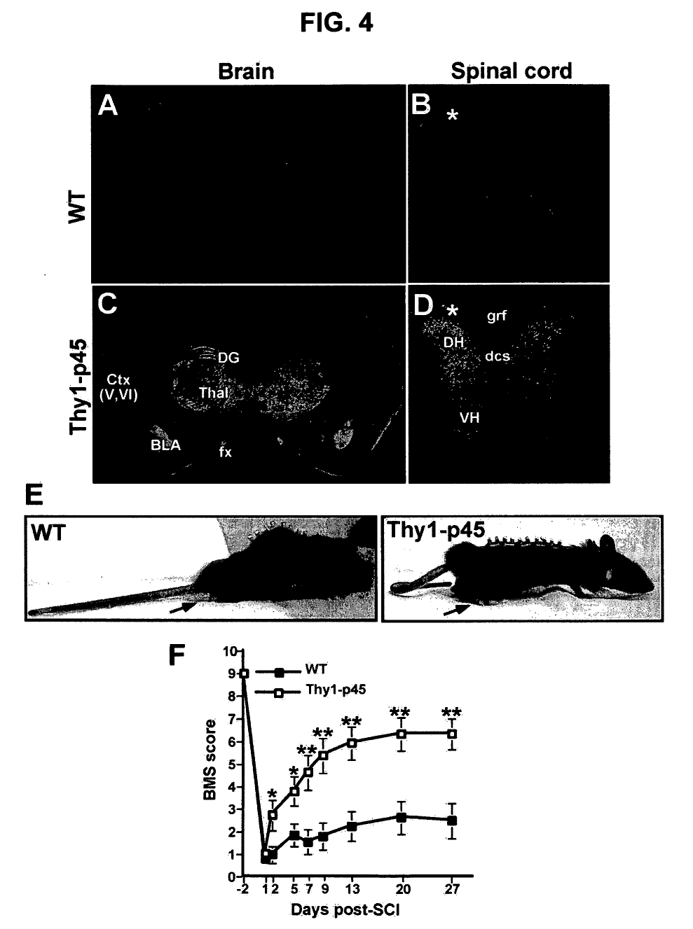

It was known that p45 expression is markedly reduced in the adult brain and spinal cord (see also FIG. 1A and FIGS. 4A and 4B). The discovery herein that p45 suppresses p75-NgR-mediated inhibition of neurite outgrowth, suggested that increasing p45 levels in adult neurons may promote spinal cord regeneration in vivo. Accordingly, transgenic mice over-expressing p45 under the control of a neuron-specific Thy1 promoter were generated.

Analysis of several lines of Thy1-p45 transgenic mice revealed that p45 was highly expressed in neuronal structures in the brain and spinal cord, including layers V and VI of the cortex, dorsal CST, and ventral and dorsal horns of the spinal cord (see FIGS. 4C and 4D). The Thy1-p45 transgenic mice did not display an overt phenotype. Rotarod test showed no differences in the motor control and/or motor learning of controls and transgenic mice.

To stringently test the role of p45 expression in recovery from spinal cord injury and avoid spared axons from dorsal hemisection of the spinal cord, which might contribute to functional recovery (Steward et al., J. Comp. Neurol., 459:1, 2003), wild type and Thy1-p45 transgenic mice (line 95) were subjected to a complete spinal cord transection at the lumbar level L1-2. The Basso Mouse Scale (BMS) (Basso and Fisher, J. Rehab. Research Devel., 40(Suppl. 3):26, 2003; Engesser-Cesar et al., J. Neurotrauma, 22:157, 2005), which is a more sensitive indicator of motor recovery in mice than is the Basso, Beattie and Bresnahan (BBB) scale, was used to assess functional recovery of injured control and Thy1-p45 transgenic mice. The BMS has a score scale from 1 to 9. As shown in FIG. 4E, as expected, control (WT) mice were completely paraplegic one week after spinal cord transection. In contrast, Thy1-p45 transgenic mice exhibited remarkable weight-bearing postures and stepping with the hindlimbs. The BMS scores revealed that Thy1-p45 transgenic mice have significant behavioral improvement even as early as 2-days post-spinal cord transection (see FIG. 4F). Locomotor performance at following each time point was significantly better in Thy1-p45 transgenic mice as compared with controls (FIG. 4f).

Similar results were obtained with wild type and Thy1-p45 transgenic mice that were subjected to a complete spinal cord transection at the thoracic level T9-10.

This Example demonstrates that over-expression of p45 in vivo promotes rapid functional recovery following a complete spinal cord transection.

### Example 6

### p45 Promotes Regeneration of the RST and CST

As shown in Example 5, p45 expression promotes a rapid improvement in locomotor function of Thy1-p45 transgenic mice following a complete spinal cord transection. This Example demonstrates nerve regeneration in at least the CST and RST in Thy1-p45 mice. Our results suggest that p45 promotes nerve regeneration by both p75/NgR-dependent and -independent mechanisms. It is believed that this disclosure the first demonstration of CST nerve regeneration by any means in a living subject following a complete spinal cord transection (e.g., when no dura is left intact).

The rapid functional recovery of Thy1-p45 transgenic mice from spinal cord transection indicated regeneration of severed descending motor pathways. Regeneration of the raphespinal tract (RST) has been shown to occur in NgR mutant mice following a complete spinal cord transection and such RST regeneration was reported to contribute to functional recovery (Kim et al., Neuron, 44:439, 2004). However, the CST did not regenerate in NgR mutants even in a hemisection paradigm (Kim et al., Neuron, 44:439, 2004; Zheng et al., Proc. Natl. Acad. Sci. USA, 102:1205-1210, 2005). For comparison to this other mouse model, regeneration of the RST and CST in the Thy1-p45 mice were examined.

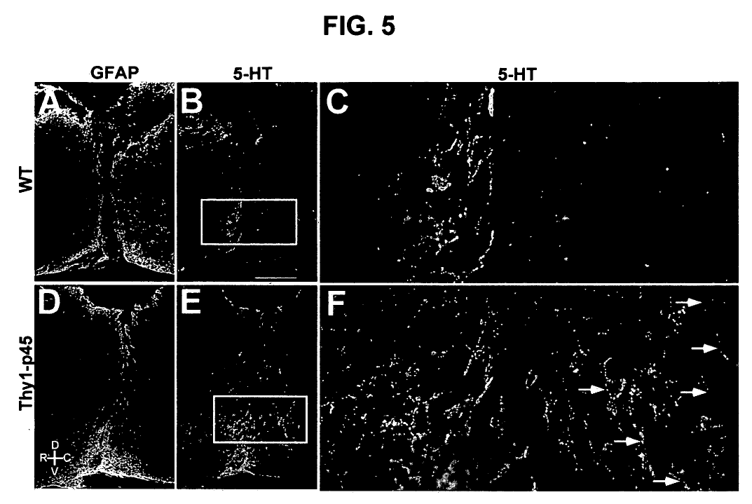

Since the RST is believed to be the only source of serotonergic input to the spinal cord (Schmidt and Jordan, Brain Res. Bull., 53:689, 2000), regeneration of the RST can be assessed by immunostaining with antibodies against hydroxytryptamine (5-HT). Parasagittal spinal cord sections were immunostained with anti-GFAP and anti-5-HT antibodies. GFAP-positive glia scars were observed in both controls and Thy1-p45 mice (see FIGS. 5A and 5D). As shown in FIGS. 5E and 5F, numerous 5-HT immunoreactive fibers were detected in regions of the spinal cord caudal to the injury site in Thy1-p45 mice. In contrast, few, if any, 5-HT immuoreactive fibers were detected in control sections (FIGS. 5B and 5C). As the RST regenerated in both NgR mutants and Thy1-p45 transgenic mice, this Example further indicates that p45 promotes nerve regeneration of the RST, at least in part, by interfering with signaling through the NgR-p75 complex (see, e.g., FIG. 2E).

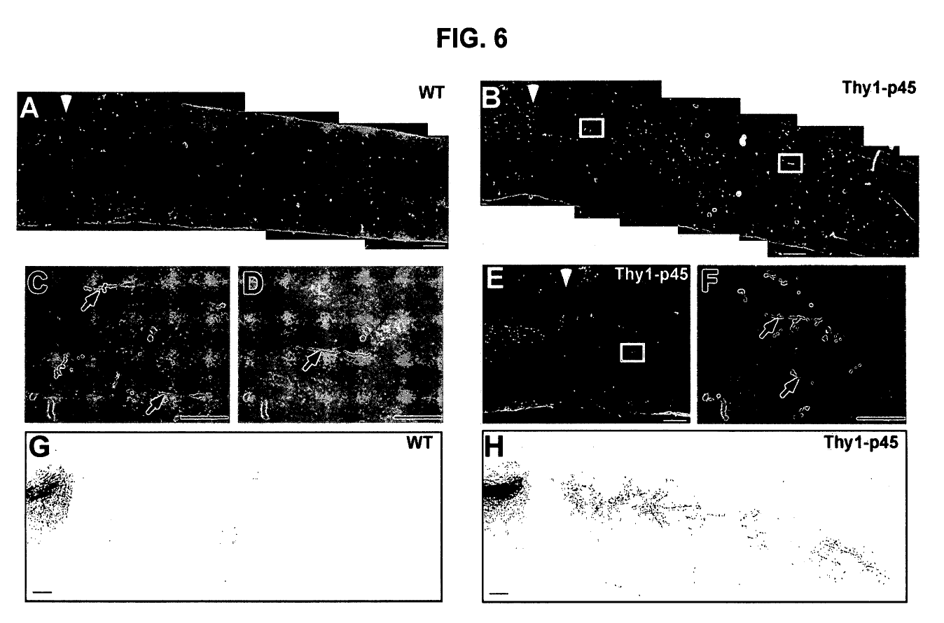

To assess regeneration of the CST, biotin-dextran amine (BDA) was injected into the sensory-motor cortex of injured mice 4 weeks post-spinal cord transection. Two weeks following BDA-injection, parasagittal sections of the spinal cords were analyzed for regenerating axons. As shown in FIGS. 6B, 6E and 6H, regenerating axons were observed caudal to the lesion site in the Thy1-p45 mice. In contrast, no regenerating axons were detected in control mice (FIGS. 6A and 6G). Higher magnification views of the regenerating CST fibers showed a morphology resembling synaptic boutons in Thy1-p45 transgenic mice (FIGS. 6C, 6D and 6F), whereas no BDA-traced CST fibers were detected in control mice. Composite drawings from serial BDA-labeled spinal cord sections showed long-distance regeneration of the CST in Thy1-p45 transgenic mice (FIG. 6H), but not in control mice (FIG. 6G). As mentioned previously, this disclosure describes the first mouse model in which regeneration of the CST occurs following a complete spinal cord transection. Because no CST regeneration was observed in NgR and p75 mutant mice (see, Kim et al., Neuron, 44:439, 2004; Zheng et al., Proc. Natl. Acad. Sci. USA, 102:1205-1210, 2005; Song et al., J. Neurosci., 24:542, 2004), this Example further indicates that p45 promotes regeneration of the CST, at least in part, by a mechanisms that does not involve the p75-NgR complex.

Although not bound by any particular theory, p75-NgR-independent mechanisms of p45 action may involve, among other things, (i) p45 cooperation with TrkA, TrkB or TrkC receptor to increase binding affinity of nerve growth factor to TrkA (Kanning et al., J. Neurosci. 23:5425, 2003; Murray et al., J. Neurosci., 24:2742, 2004), or (ii) transcriptional regulation of unknown target genes by γ-secretase cleavage fragments of the p45 ICD (Kanning et al., J. Neurosci. 23:5425, 2003; Jung et al., J. Biol. Chem., 278:42161, 2003).

### Example 7

### Representative Materials and Methods

This Example describes materials and methods used in the foregoing Examples 1-6.

A. Transfection and Immunoprecipitation

Constructs containing NgR, the full-length p45 or p75 as well as the deletion and amino acid point mutants were transfected into HEK293 cells by cytofectene transfection reagents (BIORAD) in accordance with the manufacturer's instructions.

B. RNA Transfection and Neurite Outgrowth Assay

Full-length p45 RNA with capping at 5′ end and poly (A) sequences was in vitro transcribed using the mMESSAGE-mMACHINE™ kit (AMBION) in accordance with the manufacturer's instructions. RNA was transfected into cerebellar granule neurons (CGNs) with the TRANSMESSENGER™ transfection reagent (QIAGEN) as described by the manufacturer. Neurite outgrowth assays on CGNs were carried out as previously described (Wang et al., Nature, 420:74, 2002).

C. Generation of Thy1-p45 Transgenic Mice and Spinal Cord Complete Transection

A full-length mouse p45 cDNA containing a Flag tag at the N-terminal was cloned into Xho I site of the Thy-1 expression cassette (Rockenstein et al., J. Neurosci Res., 66:573, 2001). The transgene construct was injected into one-cell embryos of DBA X C57/B6. Using appropriate aseptic techniques, 13 weeks old littermates of female controls and transgenic mice (10 each) received a dorsal laminectomy at a single thoracic vertebral segment (T9). The spinal cord was exposed and Lidocaine (2%, 1011) was applied to the dura for 1 minute to anesthetize the region to be transected. The entire depth of the spinal cord was transected with a pair of microscissors. The vertebral cavity was probed several times with fine forceps to ensure as complete a transection of the spinal cord and meninges as possible without disturbing the rostral or caudal spinal tissue. The overlaying muscle layers were sutured and the skin was stapled closed. Postoperatively, all animals were recovered on warming pads with moist food pellets on the bottom of the cage. Temperature and respiration were monitored until awakened. When the mice were alert, the cages were placed back on the housing system rack. Mice were singly housed. Bladders were manually expressed twice a day. An antibiotic (enrofloxacin, 2.27 mg/kg; BAYTRIL™) was given once a day for 6 days post-surgery.

D. Rotarod Test

The ROTAMEX™ 4/9 (COLUMBUS INSTRUMENT, OH) was used to evaluate fore- and hindlimb motor coordination and balance. Each mouse was placed on the 3.3 cm diameter rod at increasing speeds ranging from 5 rpm to 70 rpm in 3 minutes. The latency to fall off the Rotarod within this period of time was recorded. Each mouse received 3 consecutive trials. The mean latency for the 3 trials was used for the analysis.

E. Locomotor Behavior Analysis

Open field observations of locomotor behavior were scored using the Basso Mouse Scale (BMS) locomotor rating scale, which was developed specifically for mice. The BMS scale is based on a systematic analysis of locomotor recovery from spinal cord injury in mice. The characteristics of locomotor recovery are known to be different in mice and rats (which served as the model for other locomotor recovery scales) and the numeric ranking system of the BMS scale takes these differences into account. The BMS scale also includes a subscore to further differentiate between animals that are plantar stepping by awarding points for plantar stepping, coordination, paw position, trunk stability, and tail position. The scale is from 1 to 9. Use of the BMS for locomotion in mice has been shown to be a more sensitive and reliable indicator of recovery than the BBB scale (Basso and Fisher, J. Rehab. Research Devel., 40(Suppl. 3):26, 2003; Engesser-Cesar et al., J. Neurotrauma, 22:157, 2005). The surgery and behavorial analysis were performed in a double-blind manner.

F. Corticospinal Tract Tracing and Histological Analysis

Spinal cord injured mice received CST tracing at 27 days post-injury. A hole was drilled on each side of the skull overlying the sensorimotor cortex. The anterograde neuronal tracer biotin dextran amine (BDA) (10% BDA in 0.01M phosphate buffer; MOLECULAR PROBES, Eugene, Oreg.) was injected (2 μl) at 4 injection sites on each side. Two weeks after BDA injection, the animals were perfused and tissue was collected for histology. The spinal cords were sliced in 0.7 mm fragments and cut sagittally on a cryostat at 20 μm thickness. The sections were incubated with avidin-biotin-peroxidase complex, and the BDA tracer was visualized by nickel-enhanced diaminobenzidine HRP reaction (Thallmair et al., Nat. Neurosci., 1:124, 1998). All BDA-labeled fibers observed along a 10 μm thick line crossing the section width were counted at measured intervals: 0.5 mm and 2 mm above the lesion site and 5 mm below the lesion site. Due to variability in labeling, axon numbers were calculated as a percentage of the fibers seen 5 mm above the lesion, where the CST was intact (Bradbury et al., Nature, 416:636, 2002). A two-factor with replication ANOVA was used to analyze these data. Post hoc analysis was carried out using Bonferonni-corrected individual comparisons. For immunohistochemistry, spinal cord sections collected and dried on slides were blocked in buffer containing 3% normal goat serum, 2% BSA and 0.3% TRITON X-100™ before incubation with polyclonal rabbit anti-5-HT (1:500; IMMUNOSTAR). The density of fiber innervation was determined using NIH Image.

G. Protein Expression and Purification

Escherichia coli BL21(DE3) were used for recombinant expression of p45ICD. Bacterial cells were freshly transformed with the pGST-p45 expression vector, which encodes an N-terminal GST tag and a thrombin cleavage site. The pGST-p45 expression vector includes the p45 cDNA cloned in to pGEX-2T (PHARMACIA) according to the manufacturer's instructions. Two liters of M9 minimal medium containing (5NH₄)₂SO₄ and ¹³C-glucose as the sole nitrogen and carbon source respectively and ampicillin (100 μg/ml) was inoculated with 30 mL of preculture of pGST-p45-containing E. coli BL21(DE3) cells that had been grown at 37° C. overnight. At OD₆₀₀=0.6, protein expression was induced with 1 mM isopropyl L-D-galactopyranoside. The cells were harvested after 4 hours, and the pellet was resuspended in 30 ml PBS. After sonication, the cell lysate was centrifuged at 20,000 g for 30 minutes and the supernatant was incubated with Glutathione SEPHAROSE™ (AMERSHAM PHARMACIA BIOTECH) in PBS with 1 mM DTT at 4° C. for 1 hour. After several washes with PBS, the GST fusion protein was removed by thrombin digestion overnight at room temperature while still bound to the sepharose resin. The digest was incubated with benzamidine resin to remove the thrombin (SIGMA). The supernatant contained free p45ICD with a 95% purity according to SDS-PAGE. For the recombinant expression of rat p75ICD, E. coli BL21(DE3) cells were transformed with the pHis8-3p75 expression vector (which has the p75 coding sequence inserted in the pET28a (+) (NOVAGEN (EMD BIOSCIENCES) MCS), which encodes an N-terminal 22-amino acid affinity tag containing a 6-histidine sequence and a thrombin cleavage site.

Two liters of LB medium containing kanamycin (50 μg/ml) was inoculated with 30 ml of preculture of pHis8-3p75-containing E. coli BL21 (DE3) cells that had been grown at 37° C. overnight. At OD₆₀₀=0.6, protein expression was induced with 1 mM isopropyl L-D-galactopyranoside. The cells were harvested after 4 hours, and the pellet was resuspended in 30 ml buffer A (25 mM Tris-HCl, pH 8.0, 500 mM NaCl, 10 mM β-mercaptoethanol). After sonication, the cell lysate was centrifuged at 20,000 g for 30 minutes, and the supernatant was applied to a Ni2⁺-charged NTA column (QIAGEN, Chatsworth, Calif.). The fusion protein was eluted with a stepwise gradient of 0-500 mM imidazole in buffer A. After dialysis against PBS (pH 8.0), the N-terminal fusion tail was removed by thrombin cleavage as described above. Proteins were concentrated by using a 10 kDa centriprep AMICON™ concentrator. The concentrations of all proteins used in this study were determined from their absorbance at 280 nm by using molar extinction coefficients calculated from the EXPASY™ protein server software.

H. Protein Expression, NMR Spectroscopy and p45DD Modeling

The NMR experiments were carried out on a BRUKER DRX700 spectrometer at 25° C. by using protein solutions containing PBS (pH 8.0), 100 mM NaCl, 1 mM sodium azide and 10% D₂O. Sequential assignments of backbone resonances of p45ICD were made from HNCA^{coded}CB, HNCA^{coded}CO [Ritter, 2004 #6614], HNCA, ¹⁵N-resolved [¹H, ¹H]-NOESY spectra. The data were analyzed using CARA software program. For the titrations experiments, [¹⁵N, ¹H]-TROSY spectra (Pervushin et al., Proc Natl Acad Sci USA. 94(23):12366-71, 1997) were measured at a protein concentration of 0.5 mM of ¹⁵N, ¹³C-labeled p45ICD. The titration experiments were started with the 1:0 mixture of ¹³C, ¹⁵N-labeled p45ICD, and unlabeled p75ICD was added stepwise from 0.25 mM to 2 mM (protein ratios were, 1:0.5, 1:1, 1:2, and 1:4). The 3D model of a death domain region of p45 (residues 130-228 of SEQ ID NO: 2) was generated using the SWISS-MODEL protein server using the coordinates of p75DD (Liepinsh et al., EMBO J. 16(16):4999-5005, 1997) as a template.

### Example 8

### Primate p45 from Human, Chimp and Macaque are all Pseudogenes

To determine whether the p45 gene and protein is conserved in different vertebrate species, human, chimp, macaque, mouse, rat, pig, cow, dog, opossum, and frog p45 sequences, which were mined from publicly available sequence databases, were compared. These included, for rat, GENBANK™ Accession No. gi21245116; for mouse, GENBANK™ Accession No. gi22902118; for cow, the ESTs shown in GENBANK™ Accession Nos. gi6960635, gi10169399, gi6708574, gi10124689, gi10758691, gi9600878, gi6742896, gi11700620, gi12281456, gi12124371, gi15961970, and gi10072859; for pig, the ESTs shown in GENBANK™ Accession Nos. gi11582544, gi6944063, gi7047522, gi7842810, gi11075888, and gi15182544; for dog, GENBANK™ Accession No. gi57101663; for human, GENBANK™ Accession Nos. gi29150432 (human chromosome 3 clone CTD-2563A18) and gi18201839 (human chromosome 3 clone RP11-425J9); for chimp, Contig 30.78 in genome sequencing center at Washington University; and, for macaque, Mmul0.1 Contig 455144 from the rhesus macaque genome database of Human Genome Sequencing Center at Baylor College of Medicine. Surprisingly, each of the primate p45 sequences examined (i.e., from human, chimp and macaque) was a pseudogene. Each primate p45 pseudogene had a different disablement, which indicated that the p45 protein sequence was intact (e.g., no mutation, truncation, or deletion of nucleotides), at least in an early primate.

Mouse and rat p45 genes map to genomic assemblies and each gene has 5 exons. The first exon is non-coding, the second exon encodes the start of the unique extracellular region, and the other two splice sites align with the p75 paralog, cDNAs, ESTs, and genomic homologs in dog, cow, pig, opossum (Monodelphus), and two frog (Xenopus tropicalis and X. laevis) genomes. Rodent sequences were used to improve the prediction of the human form (a pseudogene) and eliminate one of the insults (a frameshift) that was originally thought to be in the gene. The human p45 gene retains one in-frame stop and one frameshift. The human gene is syntenic with mouse and has the same intron-exon structure; therefore, the human gene is a degenerate gene rather than a retrotransposed pseudogene copy. The starting sequence had one frameshift (consisting of a single nucleotide insert) and a stop in the sequence.

The chimp p45 gene sequence has lost the second splice donor site (GC). Accordingly, chimp had the same two insults as human, but also added a likely loss of a splice site. Hence, the common ancestor of human and chimp p45 was a pseudogene with a frameshift and stop.

A rhesus macaque p45 gene sequence is now publicly available (see contig Mmul0.1 Contig455144 from the rhesus macaque genome database of Human Genome Sequencing Center at Baylor College of Medicine). The rhesus macaque is a more distant relative to humans than is the chimp (i.e., ˜6% DNA diversity, compared with 1.5% in chimp, ˜25 million years of evolution vs. ˜5 million). Therefore, rhesus macaque p45 gene sequence was used to look further back in time at the degeneration of p45. The macaque p45 gene has a 3-base-pair deletion (equivalent to one amino acid), lacks the one amino acid insert seen in human and chimp to cause the frameshift, but also has two further 1 nucleotide deletions, causing two non-compensating frameshifts. Each of these events occurs in the second exon. Thus, this is not due to any error of gene prediction; although the frameshift mutation of rhesus macaque p45 gene is different from that in human and chimp, both frameshift mutations occurred in the same second exon and result to non-coding primate p45 proteins, and thus likely occurred during evolution for the non-coding p45 proteins in primates.

Together with results from improved functional recovery and nerve regeneration in Thy1-p45 transgenic mice (see Examples 5 and 6), this analysis of p45 gene sequences in divergent species suggests that the loss of functional p45 in human, chimp and macaque may contribute to a lack of, or limit the, spontaneous nerve regeneration in primates following spinal cord injury.

### Example 9

### High Concentration of p45 Protein is Detected in the Extracellular Environment of Thy1-P45 Transgenic Mice

In the course of analyzing the patterns of p45 protein expression in transgenic mice, it was unexpectedly discovered that, in addition to expression in the membrane of neurons, p45 protein immunoreactivity appeared to be in the extracellular space between neurons, axons, and glia. Since the antibodies used were specific for the intracellular domain of p45, these results indicate that heterologously expressed p45 protein may undergo a novel form of processing and trafficking. As demonstrated in the Thy1-p45 transgenic mice of Examples 5 and 6, p45 that may be (at least in part) located in the extracellular space can function to prevent degeneration and/or promote regeneration of the nervous system following spinal cord injury. This unexpected discovery indicates that exogenous p45 protein or functional p45 fragments or variants may not need to be targeted to the neuronal membrane or other intracellular compartment, but may function as potent therapeutic agents when administered in, or localized to, the extracellular space in the region of the injury.

### Example 10

### p45 Promotes Nerve Outgrowth in the Presence of Nerve Growth Inhibitors, CSPGS and Ephrin-B3

This Example demonstrates that over-expression of p45 prevents neurite-outgrowth inhibition induced by chondroitin sulphate proteoglycans (CSPGs) and ephrin-B3 (EFNB3).

Primary cerebellar granular neurons (CGNs) were isolated from P7 pups of either Thy1-p45 transgenic mice or their wild-type littermates. CGNs from Thy1-p45 transgenic mice overexpress p45 as compared to wild-type CGNs. The CGNs were plated onto coverslips coated with CSPGs, EFNB3 or PBS (control) and allowed to grow overnight. The cultures were double immunostained with rabbit anti-p45 or mouse anti-neurotubulin (TuJ1) antibodies followed by Cy3-conjugated anti-rabbit or fluorescein-conjugated anti-mouse secondary antibodies, respectively. The lengths of TuJ1-positive neurites were measured. Thy1-p45 CGNs exhibited longer neurite outgrowths as compared to CGNs from wild-type mice. Thus, CSPGs- or EFNB3-dependent inhibition of neurite-outgrowth was alleviated by p45 overexpression.

### Example 11

### p45 Binds to FADD and Inhibits Neuronal Cell Death

Examples 1 and 2 demonstrate that p45 antagonizes p75 signaling in part through the interaction of their death domains (DD); thus, it was expected that p45 may mediate its nerve regenerating activity by inhibiting cell death induced by other DD-containing molecules, including members of the TNFR family and their adaptor molecules. This Example demonstrates that p45 binds FADD, which is a universal adapter protein in apoptosis that mediates signaling of all known DD-containing members of the TNF receptor superfamily (Kabra et al., Proc. Natl. Acad. Sci. USA, 98:6307-6312, 2001). It is known that overexpression of FADD in mammalian cells induces apoptosis (Yeh et al., Science 279: 1954-1958, 1998). Although not bound by theory, it is believed that p45 binding to FADD blocks FADD-dependent death signaling and, thereby, promotes nerve cell survival at early stages following spinal cord injury.

HEK293-crmA cells stably expressing crmA, which will block the death signaling of FADD overexpression, were co-transfected with expression vectors encoding a Flag-tagged-full-length p45 and V5-tagged-full-length FADD. An anti-Flag M2 antibody (SIGMA, Cat. No. F3165) was used to immunoprecipitate p45. The immunoprecipitated material was separated by gel electrophoresis, transferred to a membrane and probed with anti-V5 antibody (INVITROGEN; Cat. No. R960-25). V5-tagged FADD was detected in the anti-Flag/Flag-tagged p45 immunoprecipitates.

Co-immunoprecipitation of FADD and p45 showed that FADD formed a stable protein complex with p45. In view of this result, the effect of p45 overexpression on FADD signaling in nerve cells was examined. Primary cultures of P7 CGNs isolated from either Thy1-p45 transgenic mice or wild-type littermates were treated with either TNF-alpha or FAS ligand, and the numbers of surviving cells were counted 1, 3 and 7 days after treatment. Treated Thy1-p45 CGNs, which overexpress p45, survived longer than treated wild-type controls.

To examine the effect of p45 on neuronal cell death following spinal cord injury in vivo, a T9/T10 complete spinal cord transection is performed in Thy1-p45 transgenic mice and their wild-type littermates. Injured spinal cords are collected 3 and 7 days following transection. Sagittal sections of injured spinal cords are stained with Fluoro-Jade C or a TUNNEL assay is performed and the number of dead neurons in the isolated Thy1-p45 or wild-type isolated spinal cords is determined. Fewer dead neuronal cells are found in transected spinal cords of Thy1-p45 transgenic mice as compared to their wild-type littermates.

### Example 12

### p45 Binds to Proteins Involved in Oxidative Metabolism, Cytoskeletal Regulation, Vesicle Transport and Oral-Facial-Digital Syndrome I

This Example demonstrates that p45 selectively binds a variety of proteins found in whole brain and spinal cord extracts.

Thy1-p45 transgenic mice express a p45 protein with an N-terminal Flag tag. Anti-Flag M2 antibody conjugated to agarose (SIGMA, Cat. No. A2220) was used to pack an affinity column. Whole brain and spinal cord extracts of Thy1-p45 transgenic mice or their wild-type littermates were collected and separately loaded onto the anti-Flag M2 antibody column. Columns were washed with 1×PBS three times and material bound to the column was eluted using 0.1 M glycine HCl, pH 3.5. The collected eluates were separated by 12% SDS-PAGE. Lanes containing electrophoresed proteins from Thy1-p45 and wild-type eluates were each cut into 10 pieces containing size-matched proteins. The respective protein samples were extracted and analyzed by mass spectrometry. The mass spectrometry results from Thy1-p45 eluates were compared to those of wild-type eluates. Proteins found only in Thy1-p45 eluates were identified to produce a p45 binding protein profile.

The proteins that selectively bound to p45 fell into four classes: (i) mitochondria membrane proteins (NADH:ubiquinone oxidoreductase (Ndufv2); ATP synthase, H⁺ transporting mitochondrial F1 complex, beta-subunit (Atp5b); dihydrolipoamide S-acetyltransferase (DLAT), and succinate dehydrogenase (Sdha)); (ii) cytoskeleton-regulating proteins (neural tropomodulin (N-Tmod, Tmod2) and rab GDP dissociation inhibitor-alpha (Rab-GDI)); (iii) vesicle transportation protein (Syntaxin binding protein, a.k.a., STXbp1, Munc18-1, p67); and (iv) Oral-facial-digital syndrome 1-related protein (OFD1).

The functions of the p45 binding partners identified in this Example indicate that p45 is involved in mitochondrial signaling (e.g., energy metabolism and/or death signaling), cytoskeleton rearrangement of the neurite outgrowth, and synaptic vesicle transportation and synaptic transmission. Interactions of Ndufv2, Atp5b, DLAT, Sdha, N-Tmod, Rab-GDI, Munc18-1, and/or OFD1 with p45 are expected to contribute to p45-dependent functional recovery following spinal cord injury.

While this disclosure has been described with an emphasis upon particular embodiments, it will be obvious to those of ordinary skill in the art that variations of the particular embodiments may be used and it is intended that the disclosure may be practiced otherwise than as specifically described herein. Accordingly, this disclosure includes all modifications encompassed within the spirit and scope of the disclosure as defined by the following claims.

1. A method for promoting nerve regeneration comprising administering to a subject a therapeutically effective amount of a p45 polypeptide or a nucleic acid encoding the p45 polypeptide.

2. The method of claim 1, wherein the p45 polypeptide is a non-primate, mammalian p45 polypeptide.

3. The method of claim 1, wherein the p45 polypeptide comprises an amino acid sequence as set forth in SEQ ID NO: 2, 4, 6, 8, or 10.

4. The method of claim 1, wherein the p45 polypeptide comprises an amino acid sequence as set forth in:
(a) an amino acid sequence as set forth in SEQ ID NO: 2;
(b) an amino acid sequence as set forth in residues 161-178 of SEQ ID NO: 2, residues 75-228 of SEQ ID NO: 2, residues 53-228 of SEQ ID NO: 2, or residues 53-221 of SEQ ID NO: 2;
(c) an amino acid sequence having at least 90% sequence identity to SEQ ID NO: 2, residues 161-178 of SEQ ID NO: 2, residues 75-228 of SEQ ID NO: 2, residues 53-228 of SEQ ID NO: 2, or residues 53-221 of SEQ ID NO: 2; wherein the amino acid sequence has nerve-regenerating activity.

5. The method of claim 1, wherein the subject has a spinal cord injury and the therapeutically effective amount is sufficient to cause a detectable improvement in the locomotor function of the subject as compared to an untreated subject.

6. The method of claim 1, wherein the therapeutically effective amount comprises from about 0.1 to about 10 mg/kg body weight.

7. A method for promoting nerve growth comprising contacting a nerve cell with a growth-promoting amount of a p45 polypeptide or a nucleic acid encoding the p 45 polypeptide.

8. The method of claim 7, wherein nerve growth comprises regeneration of the transected or crushed axon.

9. A pharmaceutical composition, comprising a pharmaceutically acceptable carrier and an isolated polypeptide having an amino acid sequence comprising LAGX₁LGYQAEAVETMA; wherein X₁ is H, Q, R, or Y.

10. The pharmaceutical composition of claim 9, wherein the amino acid sequence comprises residues 161-178 of SEQ ID NO: 2, residues 161-178 of SEQ ID NO: 4, residues 162-179 of SEQ ID NO: 6, or residues 176-193 of SEQ ID NO: 8.

11. The pharmaceutical composition of claim 10, wherein the isolated polypeptide has an amino acid sequence consisting essentially of LAGX₁LGYQAEAVETMA; wherein X₁ is H, Q, R, or Y.

12. A method of identifying an agent having potential to promote nerve regeneration, the method comprising:
contacting with at least one test agent a cell comprising a nucleic acid sequence encoding a p45 polypeptide, or a reporter gene operably linked to a p45 transcription regulatory sequence; and
detecting an increase in the expression of the p45 polypeptide or the reporter gene in the cell;
thereby identifying the at least one test agent as an agent having potential to promote nerve regeneration.

13. A method of identifying an agent having potential to promote nerve regeneration, the method comprising:
providing a first component comprising a p45 polypeptide;
providing a second component comprising a p45 specific-binding partner;
contacting the first component and the second component with at least one test agent under conditions that would permit the p45 polypeptide and the p45 specific-binding partner to bind to each other in the absence of the at least one test agent; and
determining whether the at least one test agent affects the binding of the p45 polypeptide and the p45 specific-binding partner to each other;
wherein an effect on the binding of the p45 polypeptide and the p45 specific-binding partner to each other identifies the at least one test agent as an agent having potential to promote nerve regeneration.

14. The method of claim 13, wherein the p45 specific-binding partner is a p75 polypeptide, FADD, Ndufv2, Atp5b, DLAT, Sdha, N-Tmod, Rab-GDI, Munc18-1, or OFD1.

15. The method of claim 13, wherein the p45 specific-binding partner is a p75 polypeptide.

16. The method of claim 15, further comprising determining whether the agent having potential to promote nerve regeneration specifically binds to a p75 polypeptide.

17. The method of claim 13, wherein the p45 polypeptide comprises at least 15 consecutive amino acids of SEQ ID NO: 2 or at least 15 consecutive amino acids of a polypeptide having 90% sequence identity with SEQ ID NO: 2.

18. The method of claim 13, wherein the p45 polypeptide comprises residues 161-178 of SEQ ID NO: 2, residues 161-178 of SEQ ID NO: 4, residues 162-179 of SEQ ID NO: 6, or residues 176-193 of SEQ ID NO: 8.

19. The method of claim 15, wherein the p75 polypeptide comprises at least 15 consecutive amino acids of SEQ ID NO: 18 or at least 15 consecutive amino acids of a polypeptide having 90% sequence identity with SEQ ID NO: 18.

20. The method of claim 19, wherein the p75 polypeptide comprises residues 360-377 of SEQ ID NO: 18.

21. A method of identifying an agent having potential to promote nerve outgrowth, the method comprising:
contacting with a test agent a first test system comprising a first cell that expresses first amount of a p45 polypeptide;
contacting with the same test agent a second test system comprising a second cell that expresses substantially more of the p45 polypeptide than does the first cell; and
detecting in the presence of the test agent an increased p45 function in the second cell as compared to the first cell;
wherein detection of increased p45 function in the second cell as compared to the first cell identifies the test agent as an agent having potential to promote nerve outgrowth.

22. The method of claim 21, wherein the p45 function comprises neurite outgrowth, growth cone development, or inhibition of MAG-Fc-induced RhoA.

23. The method of claim 21, wherein the first cell and the second cell further express a p75 polypeptide.
